SEQUENCE LISTING (137 sequences)

SEQ ID NO: 1 - DNA, 1758 bp, Homo sapiens
  misc_feature (1)..(1758): cDNA encoding HSA
gatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataa

SEQ ID NO: 2 - protein, 585 aa, Homo sapiens [sequence omitted]

SEQ ID NO: 3 - protein, 585 aa, Artificial Sequence
  HSA K573P
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 4 - protein, 609 aa, Homo sapiens [sequence omitted]

SEQ ID NO: 5 - protein, 621 aa, Pan troglodytes
MNESSCCSTSLPAFGVSVLDSGHSSSSAYSRGVFRRDAHKSEVAHRFKDLGEENFKALVLVAFAQYLQQCPFEDHVKLVNEVTEFAKTCVADESAENCDKSLHTLFGDKLCTVATLREKYGEMADCCAKQEPERNECFLQHKDDNPNLPRLVRPEVDVMCTAFHDNEGTFLKKYLYEVARRHPYFYAPELLFFAERYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCLAEVENDEMPADLPSLAADFVESKEVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 6 - protein, 608 aa, Macaca mulatta
MKWVTFISLLFLFSSAYSRGVFRRDTHKSEVAHRFKDLGEEHFKGLVLVAFSQYLQQCPFEEHVKLVNEVTEFAKTCVADESAENCDKSLHTLFGDKLCTVATLRETYGEMADCCAKQEPERNECFLQHKDDNPNLPPLVRPEVDVMCTAFHDNEATFLKKYLYEVARRHPYFYAPELLFFAARYKAAFAECCQAADKAACLLPKLDELRDEGKASSAKQRLKCASLQKFGDRAFKAWAVARLSQKFPKAEFAEVSKLVTDLTKVHTECCHGDLLECADDRADLAKYMCENQDSISSKLKECCDKPLLEKSHCLAEVENDEMPADLPSLAADYVESKDVCKNYAEAKDVFLGMFLYEYARRHPDYSVMLLLRLAKAYEATLEKCCAAADPHECYAKVFDEFQPLVEEPQNLVKQNCELFEQLGEYKFQNALLVRYTKKVPQVSTPTLVEVSRNLGKVGAKCCKLPEAKRMPCAEDYLSVVLNRLCVLHEKTPVSEKVTKCCTESLVNRRPCFSALELDEAYVPKAFNAETFTFHADMCTLSEKEKQVKKQTALVELVKHKPKATKEQLKGVMDNFAAFVEKCCKADDKEACFAEEGPKFVAASQAALA

SEQ ID NO: 7 - protein, 608 aa, Mesocricetus auratus
MKWVTFLLLLFVSDSAFSRGLFRRDAHKSEIAHRFKDLGEQHFKGLVLIAFSQFLQKCPYEEHVKLVNEVTDFAKTCVADESAENCDKSLHTLFGDKLCAIPTLRDSYGELADCCAKKEPERNECFLKHKDDHPNLPPFVRPDAEAMCTSFQENAVTFMGHYLHEVARRHPYFYAPELLYYAEKYSAIMTECCGEADKAACITPKLDALKEKALASSVNQRLKCSSLQRFGQRAFKAWAVARMSQKFPKADFAEITKLATDLTKLTEECCHGDLLECADDRAELAKYMCENQASISSKLQACCDKPVLKKSHCLSEVENDDLPADLPSLAADFVEDKEVCKNYAEAKDVFLGTFLYEYARRHPDYSVALLLRLAKKYEATLEKCCAEADPSACYGKVLDEFQPLVEEPKNLVKANCELFEKLGEYGFQNALIVRYTQKAPQVSTPTLVEAARNLGKVGSKCCVLPEAQRLPCVEDYISAILNRVCVLHEKTPVSEQVTKCCTGSVVERRPCFSALPVDETYVPKEFKAETFTFHADICSLPEKEKQMKKQAALVELVKHKPKATGPQLRTVLGEFTAFLDKCCKAEDKEACFSEDGPKLVASSQAALA

SEQ ID NO: 8 - protein, 608 aa, Cavia porcellus
MKWVTFISLLFLFSSVYSRGVFRREAHKSEIAHRFNDLGEGHFKGLVLITLSQHLQKSPFEEHVKLVNEVTDFAKACVADESAQNCGKAIATLFGDKVCAIPSLRETYGELADCCAKEDPDRVECFLQHKDDNPNLPPFERPEPEALCTAFKENNDRFIGHYLYEVSRRHPYFYAPELLYYAEKYKNALTECCEAADKAACLTPKLDAIKEKALVSSAQQRLKCASLQKFGERAFKAWSVARLSQKFPKAEFAEISTIVTSLTKVTKECCHGDLLECADDRQELAKYMCEHQDSISSKLKECCVKPTLQKAHCILEIQRDELPTELPDLAVDFVEDKEVCKNFAEAKDVFLGTFLYEYSRRHPEYSIGMLLRIAKGYEAKLEKCCAEADPHACYAKVFDELQPLIDEPKKLVQQNCELFDKLGEYGFQNALAVRYTQKAPQVSTPTLVEYARKLGSVGTKCCSLPETERLSCTENYLALILNRLCILHEKTPVSERVTKCCTESLVNRRPCFSALHVDETYVPKPFHADSFTFHADICTLPEKEKQVKKQMALVELVKHKPKASEEQMKTVMGDFAAFLKKCCDADNKEACFTEDGPKLVAKCQATLA

SEQ ID NO: 9 - protein, 608 aa, Mus musculus
MKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 10 - protein, 608 aa, Rattus norvegicus
MKWVTFLLLLFISGSAFSRGVFRREAHKSEIAHRFKDLGEQHFKGLVLIAFSQYLQKCPYEEHIKLVQEVTDFAKTCVADENAENCDKSIHTLFGDKLCAIPKLRDNYGELADCCAKQEPERNECFLQHKDDNPNLPPFQRPEAEAMCTSFQENPTSFLGHYLHEVARRHPYFYAPELLYYAEKYNEVLTQCCTESDKAACLTPKLDAVKEKALVAAVRQRMKCSSMQRFGERAFKAWAVARMSQRFPNAEFAEITKLATDVTKINKECCHGDLLECADDRAELAKYMCENQATISSKLQACCDKPVLQKSQCLAEIEHDNIPADLPSIAADFVEDKEVCKNYAEAKDVFLGTFLYEYSRRHPDYSVSLLLRLAKKYEATLEKCCAEGDPPACYGTVLAEFQPLVEEPKNLVKTNCELYEKLGEYGFQNAVLVRYTQKAPQVSTPTLVEAARNLGRVGTKCCTLPEAQRLPCVEDYLSAILNRLCVLHEKTPVSEKVTKCCSGSLVERRPCFSALTVDETYVPKEFKAETFTFHSDICTLPDKEKQIKKQTALAELVKHKPKATEDQLKTVMGDFAQFVDKCCKAADKDNCFATEGPNLVARSKEALA

SEQ ID NO: 11 - protein, 607 aa, Bos taurus
MKWVTFISLLLLFSSAYSRGVFRRDTHKSEIAHRFKDLGEEHFKGLVLIAFSQYLQQCPFDEHVKLVNELTEFAKTCVADESHAGCEKSLHTLFGDELCKVASLRETYGDMADCCEKQEPERNECFLSHKDDSPDLPKLKPDPNTLCDEFKADEKKFWGKYLYEIARRHPYFYAPELLYYANKYNGVFQECCQAEDKGACLLPKIETMREKVLASSARQRLRCASIQKFGERALKAWSVARLSQKFPKAEFVEVTKLVTDLTKVHKECCHGDLLECADDRADLAKYICDNQDTISSKLKECCDKPLLEKSHCIAEVEKDAIPENLPPLTADFAEDKDVCKNYQEAKDAFLGSFLYEYSRRHPEYAVSVLLRLAKEYEATLEECCAKDDPHACYSTVFDKLKHLVDEPQNLIKQNCDQFEKLGEYGFQNALIVRYTRKVPQVSTPTLVEVSRSLGKVGTRCCTKPESERMPCTEDYLSLILNRLCVLHEKTPVSEKVTKCCTESLVNRRPCFSALTPDETYVPKAFDEKLFTFHADICTLPDTEKQIKKQTALVELLKHKPKATEEQLKTVMENFVAFVDKCCAADDKEACFAVEGPKLVVSTQTALA

SEQ ID NO: 12 - protein, 607 aa, Equus caballus
MKWVTFVSLLFLFSSAYSRGVLRRDTHKSEIAHRFNDLGEKHFKGLVLVAFSQYLQQCPFEDHVKLVNEVTEFAKKCAADESAENCDKSLHTLFGDKLCTVATLRATYGELADCCEKQEPERNECFLTHKDDHPNLPKLKPEPDAQCAAFQEDPDKFLGKYLYEVARRHPYFYGPELLFHAEEYKADFTECCPADDKLACLIPKLDALKERILLSSAKERLKCSSFQNFGERAVKAWSVARLSQKFPKADFAEVSKIVTDLTKVHKECCHGDLLECADDRADLAKYICEHQDSISGKLKACCDKPLLQKSHCIAEVKEDDLPSDLPALAADFAEDKEICKHYKDAKDVFLGTFLYEYSRRHPDYSVSLLLRIAKTYEATLEKCCAEADPPACYRTVFDQFTPLVEEPKSLVKKNCDLFEEVGEYDFQNALIVRYTKKAPQVSTPTLVEIGRTLGKVGSRCCKLPESERLPCSENHLALALNRLCVLHEKTPVSEKITKCCTDSLAERRPCFSALELDEGYVPKEFKAETFTFHADICTLPEDEKQIKKQSALAELVKHKPKATKEQLKTVLGNFSAFVAKCCGREDKEACFAEEGPKLVASSQLALA

SEQ ID NO: 13 - protein, 607 aa, Equus asinus
MKWVTFVSLLFLFSSAYFRGVLRRDTHKSEIAHRFNDLGEKHFKGLVLVAFSQYLQQCPFEDHVKLVNEVTEFAKKCAADESAENCDKSLHTLFGDKLCTVATLRATYGELADCCEKQEPERNECFLTHKDDHPNLPKLKPEPDAQCAAFQEDPDKFLGKYLYEVARRHPYFYGPELLFHAEEYKADFTECCPADDKAGCLIPKLDALKERILLSSAKERLKCSSFQKFGERAFKAWSVARLSQKFPKADFAEVSKIVTDLTKVHKECCHGDLLECADDRADLTKYICEHQDSISGKLKACCDKPLLQKSHCIAEVKEDDLPSDLPALAADFAEDKEICKHYKDAKDVFLGTFLYEYSRRHPDYSVSLLLRIAKTYEATLEKCCAEADPPACYATVFDQFTPLVEEPKSLVKKNCDLFEEVGEYDFQNALIVRYTKKAPQVSTPTLVEIGRTLGKVGSRCCKLPESERLPCSENHLALALNRLCVLHEKTPVSEKITKCCTDSLAERRPCFSALELDEGYIPKEFKAETFTFHADICTLPEDEKQIKKQSALAELVKHKPKATKEQLKTVLGNFSAFVAKCCGAEDKEACFAEEGPKLVASSQLALA

SEQ ID NO: 14 - protein, 608 aa, Oryctolagus cuniculus
MKWVTFISLLFLFSSAYSRGVFRREAHKSEIAHRFNDVGEEHFIGLVLITFSQYLQKCPYEEHAKLVKEVTDLAKACVADESAANCDKSLHDIFGDKICALPSLRDTYGDVADCCEKKEPERNECFLHHKDDKPDLPPFARPEADVLCKAFHDDEKAFFGHYLYEVARRHPYFYAPELLYYAQKYKAILTECCEAADKGACLTPKLDALEGKSLISAAQERLRCASIQKFGDRAYKAWALVRLSQRFPKADFTDISKIVTDLTKVHKECCHGDLLECADDRADLAKYMCEHQETISSHLKECCDKPILEKAHCIYGLHNDETPAGLPAVAEEFVEDKDVCKNYEEAKDLFLGKFLYEYSRRHPDYSVVLLLRLGKAYEATLKKCCATDDPHACYAKVLDEFQPLVDEPKNLVKQNCELYEQLGDYNFQNALLVRYTKKVPQVSTPTLVEISRSLGKVGSKCCKHPEAERLPCVEDYLSVVLNRLCVLHEKTPVSEKVTKCCSESLVDRRPCFSALGPDETYVPKEFNAETFTFHADICTLPETERKIKKQTALVELVKHKPHATNDQLKTVVGEFTALLDKCCSAEDKEACFAVEGPKLVESSKATLG

SEQ ID NO: 15 - protein, 583 aa, Capra hircus
DTHKSEIAHRFNDLGEENFQGLVLIAFSQYLQQCPFDEHVKLVKELTEFAKTCVADESHAGCDKSLHTLFGDELCKVATLRETYGDMADCCEKQEPERNECFLKHKDDSPDLPKLKPEPDTLCAEFKADEKKFWGKYLYEVARRHPYFYAPELLYYANKYNGVFQECCQAEDKGACLLPKIETMREKVLASSARQRLRCASIQKFGERALKAWSVARLSQKFPKADFTDVTKIVTDLTKVHKECCHGDLLECADDRADLAKYICDHQDTLSSKLKECCDKPVLEKSHCIAEIDKDAVPENLPPLTADFAEDKEVCKNYQEAKDVFLGSFLYEYSRRHPEYAVSVLLRLAKEYEATLEDCCAKEDPHACYATVFDKLKHLVDEPQNLIKKNCELFEKHGEYGFQNALIVRYTRKAPQVSTPTLVEISRSLGKVGTKCCAKPESERMPCTEDYLSLILNRLCVLHEKTPVSEKVTKCCTESLVNRRPCFSDLTLDETYVPKPFDGESFTFHADICTLPDTEKQIKKQTALVELLKHKPKATDEQLKTVMENFVAFVDKCCAADDKEGCFLLEGPKLVASTQAALA

SEQ ID NO: 16 - protein, 607 aa, Ovis aries
MKWVTFISLLLLFSSAYSRGVFRRDTHKSEIAHRFNDLGEENFQGLVLIAFSQYLQQCPFDEHVKLVKELTEFAKTCVADESHAGCDKSLHTLFGDELCKVATLRETYGDMADCCEKQEPERNECFLNHKDDSPDLPKLKPEPDTLCAEFKADEKKFWGKYLYEVARRHPYFYAPELLYYANKYNGVFQECCQAEDKGACLLPKIDAMREKVLASSARQRLRCASIQKFGERALKAWSVARLSQKFPKADFTDVTKIVTDLTKVHKECCHGDLLECADDRADLAKYICDHQDALSSKLKECCDKPVLEKSHCIAEVDKDAVPENLPPLTADFAEDKEVCKNYQEAKDVFLGSFLYEYSRRHPEYAVSVLLRLAKEYEATLEDCCAKEDPHACYATVFDKLKHLVDEPQNLIKKNCELFEKHGEYGFQNALIVRYTRKAPQVSTPTLVEISRSLGKVGTKCCAKPESERMPCTEDYLSLILNRLCVLHEKTPVSEKVTKCCTESLVNRRPCFSDLTLDETYVPKPFDEKFFTFHADICTLPDTEKQIKKQTALVELLKHKPKATDEQLKTVMENFVAFVDKCCAADDKEGCFVLEGPKLVASTQAALA

SEQ ID NO: 17 - protein, 608 aa, canis lupus familiaris
MKWVTFISLFFLFSSAYSRGLVRREAYKSEIAHRYNDLGEEHFRGLVLVAFSQYLQQCPFEDHVKLAKEVTEFAKACAAEESGANCDKSLHTLFGDKLCTVASLRDKYGDMADCCEKQEPDRNECFLAHKDDNPGFPPLVAPEPDALCAAFQDNEQLFLGKYLYEIARRHPYFYAPELLYYAQQYKGVFAECCQAADKAACLGPKIEALREKVLLSSAKERFKCASLQKFGDRAFKAWSVARLSQRFPKADFAEISKVVTDLTKVHKECCHGDLLECADDRADLAKYMCENQDSISTKLKECCDKPVLEKSQCLAEVERDELPGDLPSLAADFVEDKEVCKNYQEAKDVFLGTFLYEYARRHPEYSVSLLLRLAKEYEATLEKCCATDDPPTCYAKVLDEFKPLVDEPQNLVKTNCELFEKLGEYGFQNALLVRYTKKAPQVSTPTLVEVSRKLGKVGTKCCKKPESERMSCAEDFLSVVLNRLCVLHEKTPVSERVTKCCSESLVNRRPCFSGLEVDETYVPKEFNAETFTFHADLCTLPEAEKQVKKQTALVELLKHKPKATDEQLKTVMGDFGAFVEKCCAAENKEGCFSEEGPKLVAAAQAALV

SEQ ID NO: 18 - protein, 615 aa, Gallus gallus
MKWVTLISFIFLFSSATSRNLQRFARDAEHKSEIAHRYNDLKEETFKAVAMITFAQYLQRCSYEGLSKLVKDVVDLAQKCVANEDAPECSKPLPSIILDEICQVEKLRDSYGAMADCCSKADPERNECFLSFKVSQPDFVQPYQRPASDVICQEYQDNRVSFLGHFIYSVARRHPFLYAPAILSFAVDFEHALQSCCKESDVGACLDTKEIVMREKAKGVSVKQQYFCGILKQFGDRVFQARQLIYLSQKYPKAPFSEVSKFVHDSIGVHKECCEGDMVECMDDMARMMSNLCSQQDVFSGKIKDCCEKPIVERSQCIMEAEFDEKPADLPSLVEKYIEDKEVCKSFEAGHDAFMAEFVYEYSRRHPEFSIQLIMRIAKGYESLLEKCCKTDNPAECYANAQEQLNQHIKETQDVVKTNCDLLHDHGEADFLKSILIRYTKKMPQVPTDLLLETGKKMTTIGTKCCQLGEDRRMACSEGYLSIVIHDTCRKQETTPINDNVSQCCSQLYANRRPCFTAMGVDTKYVPPPFNPDMFSFDEKLCSAPAEEREVGQMKLLINLIKRKPQMTEEQIKTIADGFTAMVDKCCKQSDINTCFGEEGANLIVQSRATLGIGA

SEQ ID NO: 19 - protein, 607 aa, Sus scrofa
MKWVTFISLLFLFSSAYSRGVFRRDTYKSEIAHRFKDLGEQYFKGLVLIAFSQHLQQCPYEEHVKLVREVTEFAKTCVADESAENCDKSIHTLFGDKLCAIPSLREHYGDLADCCEKEEPERNECFLQHKNDNPDIPKLKPDPVALCADFQEDEQKFWGKYLYEIARRHPYFYAPELLYYAIIYKDVFSECCQAADKAACLLPKIEHLREKVLTSAAKQRLKCASIQKFGERAFKAWSLARLSQRFPKADFTEISKIVTDLAKVHKECCHGDLLECADDRADLAKYICENQDTISTKLKECCDKPLLEKSHCIAEAKRDELPADLNPLEHDFVEDKEVCKNYKEAKHVFLGTFLYEYSRRHPDYSVSLLLRIAKIYEATLEDCCAKEDPPACYATVFDKFQPLVDEPKNLIKQNCELFEKLGEYGFQNALIVRYTKKVPQVSTPTLVEVARKLGLVGSRCCKRPEEERLSCAEDYLSLVLNRLCVLHEKTPVSEKVTKCCTESLVNRRPCFSALTPDETYKPKEFVEGTFTFHADLCTLPEDEKQIKKQTALVELLKHKPHATEEQLRTVLGNFAAFVQKCCAAPDHEACFAVEGPKFVIEIRGILA

SEQ ID NO: 20 - protein, 584 aa, artificial sequence
  N terminal is residues 1 to 572 of HSA. C terminal is residues 573 to 584 of Macaque albumin.
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKFVAASQAALA

SEQ ID NO: 21 - protein, 584 aa, Artificial Sequence
  N terminal is residues 1 to 572 from HSA. C terminal is residues 573 to 584 from mouse albumin.
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPNLVTRCKDALA

SEQ ID NO: 22 - protein, 584 aa, Artificial Sequence
  N terminal is residues 1 to 572 of HSA. C terminal is residues 573 to 584 of rabbit albumin.
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVESSKATLG

SEQ ID NO: 23 - protein, 584 aa, Artificial Sequence
  N-terminal is residues 1 to 572 of HSA. C-terminal is residues 573 to 583 of sheep albumin.
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVASTQAALA

SEQ ID NO: 24 - protein, 399 aa, Artificial sequence
  Artificial albumin variant human serum albumin domain 1 and human serum albumin domain 3
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 25 - protein, 403 aa, Artificial sequence
  Artificial albumin variant human serum albumin domain 2 and human serum albumin domain 3
DELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 26 - protein, 410 aa, Artificial sequence
  Artificial albumin variant two consecutive copies of human serum albumin domain 3
VEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 27 - protein, 205 aa, Artificial sequence [sequence omitted]

SEQ ID NO: 28 - protein, 615 aa, Artificial Sequence
  HSA Domain III + HSA Domain III + HSA Domain III
VEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 29 - protein, 604 aa, Artificial Sequence
  HSA Domain I + HSA Domain III + HSA Domain III
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 30 - protein, 290 aa, Homo sapiens
  misc_feature (1)..(290): Truncated heavy chain of the major histocompatibility complex class I-like Fc receptor (FCGRT) (together, SEQ ID No. 30 and SEQ ID No. 31 form FcRN)
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVEL

SEQ ID NO: 31 - protein, 119 aa, Homo sapiens
  misc_feature (1)..(119): Beta-2-microglobulin (together, SEQ ID No. 30 and SEQ ID No. 31 form FcRN)
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 32 - protein, 2059 aa, Artificial Sequence
  Synthetic DNA fragment containing 3′ region of PRB1 promoter, modified fusion leader sequence, nucleotide sequence encoding HSA and 5′ region of modified ADH1
GGTCTCAAGCTAACCTAATTCTAACAAGCAAAGATGAAGTGGGTTTTCATCGTCTCCATTTTGTTCTTGTTCTCCTCTGCTTACTCTAGATCTTTGGATAAGAGAGACGCTCACAAGTCCGAAGTCGCTCACAGATTCAAGGACTTGGGTGAAGAAAACTTCAAGGCTTTGGTCTTGATCGCTTTCGCTCAATACTTGCAACAATGTCCATTCGAAGATCACGTCAAGTTGGTCAACGAAGTTACCGAATTCGCTAAGACTTGTGTTGCTGACGAATCCGCGGAAAACTGTGACAAGTCCTTGCACACCTTGTTCGGTGATAAGTTGTGTACTGTTGCTACCTTGAGAGAAACCTACGGTGAAATGGCTGACTGTTGTGCTAAGCAAGAACCAGAAAGAAACGAATGTTTCTTGCAACACAAGGACGACAACCCAAACTTGCCAAGATTGGTTAGACCAGAAGTTGACGTCATGTGTACTGCTTTCCACGACAACGAAGAAACCTTCTTGAAGAAGTACTTGTACGAAATTGCTAGAAGACACCCATACTTCTACGCTCCAGAATTGTTGTTCTTCGCTAAGAGATACAAGGCTGCTTTCACCGAATGTTGTCAAGCTGCTGATAAGGCTGCTTGTTTGTTGCCAAAGTTGGATGAATTGAGAGACGAAGGTAAGGCTTCTTCCGCTAAGCAAAGATTGAAGTGTGCTTCCTTGCAAAAGTTCGGTGAAAGAGCTTTCAAGGCTTGGGCTGTCGCTAGATTGTCTCAAAGATTCCCAAAGGCTGAATTCGCTGAAGTTTCTAAGTTGGTTACTGACTTGACTAAGGTTCACACTGAATGTTGTCACGGTGACTTGTTGGAATGTGCTGATGACAGAGCTGACTTGGCTAAGTACATCTGTGAAAACCAAGACTCTATCTCTTCCAAGTTGAAGGAATGTTGTGAAAAGCCATTGTTGGAAAAGTCTCACTGTATTGCTGAAGTTGAAAACGATGAAATGCCAGCTGACTTGCCATCTTTGGCTGCTGACTTCGTTGAATCTAAGGACGTTTGTAAGAACTACGCTGAAGCTAAGGACGTCTTCTTGGGTATGTTCTTGTACGAATACGCTAGAAGACACCCAGACTACTCCGTTGTCTTGTTGTTGAGATTGGCTAAGACCTACGAAACTACCCTCGAGAAGTGTTGTGCTGCTGCTGACCCACACGAATGTTACGCTAAGGTTTTCGATGAATTCAAGCCATTGGTCGAAGAACCACAAAACTTGATCAAGCAAAACTGTGAATTGTTCGAACAATTGGGTGAATACAAGTTCCAAAACGCTTTGTTGGTTAGATACACTAAGAAGGTCCCACAAGTCTCCACCCCAACTTTGGTTGAAGTCTCTAGAAACTTGGGTAAGGTCGGTTCTAAGTGTTGTAAGCACCCAGAAGCTAAGAGAATGCCATGTGCTGAAGATTACTTGTCCGTCGTTTTGAACCAATTGTGTGTTTTGCACGAAAAGACCCCAGTCTCTGATAGAGTCACCAAGTGTTGTACTGAATCTTTGGTTAACAGAAGACCATGTTTCTCTGCTTTGGAAGTCGACGAAACTTACGTTCCAAAGGAATTCAACGCTGAAACTTTCACCTTCCACGCTGATATCTGTACCTTGTCCGAAAAGGAAAGACAAATTAAGAAGCAAACTGCTTTGGTTGAATTGGTCAAGCACAAGCCAAAGGCTACTAAGGAACAATTGAAGGCTGTCATGGATGATTTCGCTGCTTTCGTTGAAAAGTGTTGTAAGGCTGATGATAAGGAAACTTGTTTCGCTGAAGAAGGTAAGAAGTTGGTCGCTGCTTCCCAAGCTGCCTTAGGTTTGTAATAAGCTTAATTAATATGATTTATGATTTTTATTATTAAATAAGTTATAAAAAAAATAAGTGTATACAAATTTTAAAGTGACTCTTAGGTTTTAAAACGAAAATTCTTATTCTTGAGTAACTCTTTCCTGTAGGTCAGGTTGCTTTCTCAGGTATAGCATGAGGTCGCTCTTATTGACCACACCTCTACCGGCATGC

SEQ ID NO: 33 - DNA, 36 bp, Artificial Sequence
  Oligonucleotide primer K195A Reverse
agcggaagaagccttaccttcgtctctcaattcatc

SEQ ID NO: 34 - DNA, 45 bp, Artificial Sequence
  Oligonucleotide primer K195A Forward
gaaggtaaggcttcttccgctgctcaaagattgaagtgtgcttcc

SEQ ID NO: 35 - DNA, 36 bp, Artificial Sequence
  Oligonucleotide primer L198A Reverse
tctttgcttagcggaagaagccttaccttcgtctct

SEQ ID NO: 36 - DNA, 45 bp, Artificial Sequence
  Oligonucleotide primer L198A Forward
gcttcttccgctaagcaaagagctaagtgtgcttccttgcaaaag

SEQ ID NO: 37 - DNA, 36 bp, Artificial Sequence
  Oligonucleotide primer F206A Reverse
cttttgcaaggaagcacacttcaatctttgcttagc

SEQ ID NO: 38 - DNA, 45 bp, Artificial Sequence
  Oligonucleotide primer F206A Forward
aagtgtgcttccttgcaaaaggctggtgaaagagctttcaaggct

SEQ ID NO: 39 - DNA, 36 bp, Artificial Sequence
  Oligonucleotide primer D340Y Reverse
tgggtgtcttctagcgtattcgtacaagaacatacc

SEQ ID NO: 40 - DNA, 45 bp, Artificial Sequence
  Oligonucleotide primer D340Y Forward
gaatacgctagaagacacccatattactccgttgtcttgttgttg

SEQ ID NO: 41 - DNA, 36 bp, Artificial Sequence
  Oligonucleotide primer Y341F Reverse
gtctgggtgtcttctagcgtattcgtacaagaacat

SEQ ID NO: 42 - DNA, 45 bp, Artificial Sequence
  Oligonucleotide primer Y341F Forward
tacgctagaagacacccagacttttccgttgtcttgttgttgaga

SEQ ID NO: 43 - DNA, 36 bp, Artificial Sequence
  Oligonucleotide primer S342Y Reverse
gtagtctgggtgtcttctagcgtattcgtacaagaa

SEQ ID NO: 44 - DNA, 45 bp, Artificial Sequence
  Oligonucleotide primer S342Y Forward
gctagaagacacccagactactatgttgtcttgttgttgagattg

SEQ ID NO: 45 - DNA, 36 bp, Artificial Sequence
  Oligonucleotide primer V343A Reverse
ggagtagtctgggtgtcttctagcgtattcgtacaa

SEQ ID NO: 46 - DNA, 45 bp, Artificial Sequence
  Oligonucleotide primer V343A Forward
agaagacacccagactactccgctgtcttgttgttgagattggct

SEQ ID NO: 47 - DNA, 36 bp, Artificial Sequence
  Oligonucleotide primer V344P Reverse
aacggagtagtctgggtgtcttctagcgtattcgta

SEQ ID NO: 48 - DNA, 45 bp, Artificial Sequence
  Oligonucleotide primer V344P Forward
agacacccagactactccgttccattgttgttgagattggctaag

SEQ ID NO: 49 - DNA, 36 bp, Artificial Sequence
  Oligonucleotide primer L345E Reverse
gacaacggagtagtctgggtgtcttctagcgtattc

SEQ ID NO: 50 - DNA, 45 bp, Artificial Sequence
  Oligonucleotide primer L345E Forward
cacccagactactccgttgtcgaattgttgagattggctaagacc

SEQ ID NO: 51 - DNA, 36 bp, Artificial Sequence
  Oligonucleotide primer R348F Reverse
caacaacaagacaacggagtagtctgggtgtcttct

SEQ ID NO: 52 - DNA, 45 bp, Artificial Sequence
  Oligonucleotide primer R348F Forward
tactccgttgtcttgttgttgtttttggctaagacctacgaaact

SEQ ID NO: 53 - DNA, 36 bp, Artificial Sequence
  Oligonucleotide primer L349F Reverse
tctcaacaacaagacaacggagtagtctgggtgtct

SEQ ID NO: 54 - DNA, 45 bp, Artificial Sequence
  Oligonucleotide primer L349F Forward
tccgttgtcttgttgttgagatttgctaagacctacgaaactacc

SEQ ID NO: 55 - DNA, 36 bp, Artificial Sequence
  Oligonucleotide primer T352R Reverse
cttagccaatctcaacaacaagacaacggagtagtc

SEQ ID NO: 56 - DNA, 45 bp, Artificial Sequence
  Oligonucleotide primer T352R Forward
ttgttgttgagattggctaagagatacgaaactaccctcgagaag

SEQ ID NO: 57 - DNA, 36 bp, Artificial Sequence
  Oligonucleotide primer F377L Reverse
ttcatcgaaaaccttagcgtaacattcgtgtgggtc

SEQ ID NO: 58 - DNA, 45 bp, Artificial Sequence
  Oligonucleotide primer F377L Forward
tacgctaaggttttcgatgaattgaagccattggtcgaagaacca

SEQ ID NO: 59 - DNA, 36 bp, Artificial Sequence
  Oligonucleotide primer K378R Reverse
gaattcatcgaaaaccttagcgtaacattcgtgtgg

SEQ ID NO: 60 - DNA, 45 bp, Artificial Sequence
  Oligonucleotide primer K378R Forward
gctaaggttttcgatgaattcagaccattggtcgaagaaccacaa

SEQ ID NO: 61 - DNA, 36 bp, Artificial Sequence
  Oligonucleotide primer P379D Reverse
cttgaattcatcgaaaaccttagcgtaacattcgtg

SEQ ID NO: 62 - DNA, 45 bp, Artificial Sequence
  Oligonucleotide primer P379D Forward
aaggttttcgatgaattcaaggatttggtcgaagaaccacaaaac

SEQ ID NO: 63 - DNA, 36 bp, Artificial Sequence
  Oligonucleotide primer L380E Reverse
tggcttgaattcatcgaaaaccttagcgtaacattc

SEQ ID NO: 64 - DNA, 45 bp, Artificial Sequence
  Oligonucleotide primer L380E Forward
gttttcgatgaattcaagccagaagtcgaagaaccacaaaacttg

SEQ ID NO: 65 - DNA, 36 bp, Artificial Sequence
  Oligonucleotide primer V381G Reverse
caatggcttgaattcatcgaaaaccttagcgtaaca

SEQ ID NO: 66 - DNA, 45 bp, Artificial Sequence
  Oligonucleotide primer V381G Forward
ttcgatgaattcaagccattgggtgaagaaccacaaaacttgatc

SEQ ID NO: 67 - DNA, 36 bp, Artificial Sequence
  Oligonucleotide primer E382K Reverse
gaccaatggcttgaattcatcgaaaaccttagcgta

SEQ ID NO: 68 - DNA, 45 bp, Artificial Sequence
  Oligonucleotide primer E382K Forward
gatgaattcaagccattggtcaaagaaccacaaaacttgatcaag

SEQ ID NO: 69 - DNA, 36 bp, Artificial Sequence
  Oligonucleotide primer E383A Reverse
ttcgaccaatggcttgaattcatcgaaaaccttagc

SEQ ID NO: 70 - DNA, 45 bp, Artificial Sequence
  Oligonucleotide primer E383A Forward
gaattcaagccattggtcgaagctccacaaaacttgatcaagcaa

SEQ ID NO: 71 - DNA, 36 bp, Artificial Sequence
  Oligonucleotide primer P384S Reverse
ttcttcgaccaatggcttgaattcatcgaaaacctt

SEQ ID NO: 72 - DNA, 45 bp, Artificial Sequence
  Oligonucleotide primer P384S Forward
ttcaagccattggtcgaagaatctcaaaacttgatcaagcaaaac

SEQ ID NO: 73 - protein, 585 aa, Artificial Sequence
  HSA K195A
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAAQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 74 - protein, 585 aa, Artificial Sequence
  HSA L198A
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRAKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 75 - protein, 585 aa, Artificial Sequence
  HSA F206A
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKAGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 76 - protein, 585 aa, Artificial Sequence
  HSA D340Y
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPYYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 77 - protein, 585 aa, Artificial Sequence
  HSA Y341F
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDFSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 78 - protein, 585 aa, Artificial Sequence
  HSA S342Y
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYYVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 79 - protein, 585 aa, Artificial Sequence
  HSA V343A
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSAVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 80 - protein, 585 aa, Artificial Sequence
  HSA V344P
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVPLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 81 - protein, 585 aa, Artificial Sequence
  HSA L345E
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVELLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 82 - protein, 585 aa, Artificial Sequence
  HSA R348F
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLFLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 83 - protein, 585 aa, Artificial Sequence
  HSA L349F
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRFAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 84 - protein, 585 aa, Artificial Sequence
  HSA T352R
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKRYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 85 - protein, 585 aa, Artificial Sequence
  HSA F377L
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDELKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 86 - protein, 585 aa, Artificial Sequence
  HSA K378R
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFRPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 87 - protein, 585 aa, Artificial Sequence
  HSA P379D
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKDLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 88 - protein, 585 aa, Artificial Sequence
  HSA L380E
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPEVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 89 - protein, 585 aa, Artificial Sequence
  HSA V381G
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLGEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 90 - protein, 585 aa, Artificial Sequence
  HSA E382K
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVKEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 91 - protein, 585 aa, Artificial Sequence
  HSA E383A
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEAPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 92 - protein, 585 aa, Artificial Sequence
  HSA P384S
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEESQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 93 - protein, 585 aa, Artificial Sequence
  HSA S342Y K573P
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYYVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 94 - protein, 585 aa, Artificial Sequence
  HSA L345E K573P
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVELLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 95 - protein, 585 aa, Artificial Sequence
  HSA L349F K573P
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRFAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 96 - protein, 585 aa, Artificial Sequence
  HSA V381G K573P
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLGEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 97 - protein, 387 aa, Artificial Sequence
  Artificial albumin variant human serum albumin domain 1 and 2
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNL

SEQ ID NO: 98 - protein, 585 aa, Artificial Sequence
  HSA K205A
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQAFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 99 - DNA, 45 bp, Artificial Sequence
  Oligonucleotide primer K205A Forward
ttgaagtgtgcttccttgcaagctttcggtgaaagagctttcaag

SEQ ID NO: 100 - DNA, 36 bp, Artificial Sequence
  Oligonucleotide primer K205A Reverse
ttgcaaggaagcacacttcaatctttgcttagcgga

SEQ ID NO: 101 - DNA, 45 bp, Artificial Sequence
  Oligonucleotide primer MDH30001
actgttgctaccttgagagaaaattacggtgaaatggctgactgt

SEQ ID NO: 102 - DNA, 36 bp, Artificial Sequence
  Oligonucleotide primer MDH30002
ttctctcaaggtagcaacagtacacaacttatcacc

SEQ ID NO: 103 - protein, 585 aa, Artificial Sequence
  HSA T83N S342Y K573P
DAHKSEVAHRFKDLGEENFKALVLIAFAQYLQQCPFEDHVKLVNEVTEFAKTCVADESAENCDKSLHTLFGDKLCTVATLRENYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYYVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 104 - protein, 585 aa, Artificial Sequence
  HSA T83N L349F K573P
DAHKSEVAHRFKDLGEENFKALVLIAFAQYLQQCPFEDHVKLVNEVTEFAKTCVADESAENCDKSLHTLFGDKLCTVATLRENYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRFAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 105 - protein, 585 aa, Artificial Sequence
  HSA T83N V381G K573P
DAHKSEVAHRFKDLGEENFKALVLIAFAQYLQQCPFEDHVKLVNEVTEFAKTCVADESAENCDKSLHTLFGDKLCTVATLRENYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLGEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 106 - DNA, 902 bp, Artificial Sequence
  Synthetic DNA fragment for construction of pDB5102
ccgcggaaaactgtgacaagtccttgcacaccttgttcggtgataagttgtgtactgttgctaccttgagagaaacctacggtgaaatggctgactgttgtgctaagcaagaaccagaaagaaacgaatgtttcttgcaacacaaggacgacaacccaaacttgccaagattggttagaccagaagttgacgtcatgtgtactgctttccacgacaacgaagaaaccttcttgaagaagtacttgtacgaaattgctagaagacacccatacttctacgctccagaattgttgttcttcgctaagagatacaaggctgctttcaccgaatgttgtcaagctgctgataaggctgcttgtttgttgccaaagttggatgaattgagagacgaaggtaaggctagctccgcaaagcaaagattgaagtgtgcttccttgcaaaagttcggtgaaagagctttcaaggcttgggctgtcgctagattgtctcaaagattcccaaaggctgaattcgctgaagtttctaagttggttactgacttgactaaggttcacactgaatgttgtcacggtgacttgttggaatgtgctgatgacagagctgacttggctaagtacatctgtgaaaaccaagactctatctcttccaagttgaaggaatgttgtgaaaagccattgttggaaaagtctcactgtattgctgaagttgaaaacgatgaaatgccagctgacttgccatctttggctgctgacttcgttgaatctaaggacgtttgtaagaactacgctgaagctaaggacgtcttcttgggtatgttcttgtacgaatacgctagaagacacccagactactccgttgtcttgttgttgagattggctaagacctacgaaactaccctcgag

SEQ ID NO: 107 - DNA, 48 bp, Artificial Sequence
  Oligonucleotide primer HSAJ-39
cgctagaagacacccagactacgctgttgtcttgttgttgagattggc

SEQ ID NO: 108 - DNA, 48 bp, Artificial Sequence
  Oligonucleotide primer HSAJ-40
cgctagaagacacccagactactgtgttgtcttgttgttgagattggc

SEQ ID NO: 109 - DNA, 48 bp, Artificial Sequence
  Oligonucleotide primer HSAJ-45
cgctagaagacacccagactaccacgttgtcttgttgttgagattggc

SEQ ID NO: 110 - DNA, 48 bp, Artificial Sequence
  Oligonucleotide primer HSAJ-46
cgctagaagacacccagactacatcgttgtcttgttgttgagattggc

SEQ ID NO: 111 - DNA, 48 bp, Artificial Sequence
  Oligonucleotide primer HSAJ-48
cgctagaagacacccagactacttggttgtcttgttgttgagattggc

SEQ ID NO: 112 - DNA, 48 bp, Artificial Sequence
  Oligonucleotide primer HSAJ-50
cgctagaagacacccagactacaacgttgtcttgttgttgagattggc

SEQ ID NO: 113 - DNA, 48 bp, Artificial Sequence
  Oligonucleotide primer HSAJ-51
cgctagaagacacccagactacccagttgtcttgttgttgagattggc

SEQ ID NO: 114 - DNA, 48 bp, Artificial Sequence
  Oligonucleotide primer HSAJ-52
cgctagaagacacccagactaccaagttgtcttgttgttgagattggc

SEQ ID NO: 115 - DNA, 48 bp, Artificial Sequence
  Oligonucleotide primer HSAJ-55
cgctagaagacacccagactacgttgttgtcttgttgttgagattggc

SEQ ID NO: 116 - DNA, 35 bp, Artificial Sequence
  Oligonucleotide primer HSAJ-39rev
gtagtctgggtgtcttctagcgtattcgtacaaga

SEQ ID NO: 117 - DNA, 48 bp, Artificial Sequence
  Oligonucleotide primer HSAK-07
cacccagactactccgttgtccacttgttgagattggctaagacctac

SEQ ID NO: 118 - DNA, 48 bp, Artificial Sequence
  Oligonucleotide primer HSAK-08
cacccagactactccgttgtcatcttgttgagattggctaagacctac

SEQ ID NO: 119 - DNA, 48 bp, Artificial Sequence
  Oligonucleotide primer HSAK-13
cacccagactactccgttgtccaattgttgagattggctaagacctac

SEQ ID NO: 120 - DNA, 33 bp, Artificial Sequence
  Oligonucleotide primer HSAK-01rev
gacaacggagtagtctgggtgtcttctagcgta

SEQ ID NO: 121 - DNA, 48 bp, Artificial Sequence
  Oligonucleotide primer HSAK-23
actccgttgtcttgttgttgagagaagctaagacctacgaaactaccc

SEQ ID NO: 122 - DNA, 48 bp, Artificial Sequence
  Oligonucleotide primer HSAK-32
actccgttgtcttgttgttgagacaagctaagacctacgaaactaccc

SEQ ID NO: 123 - DNA, 34 bp, Artificial Sequence
  Oligonucleotide primer HSAK-20rev
tctcaacaacaagacaacggagtagtctgggtgt

SEQ ID NO: 124 - protein, 585 aa, Artificial Sequence
  HSA S342A
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYAVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 125 - protein, 585 aa, Artificial Sequence
  HSA S342C
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYCVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 126 - protein, 585 aa, Artificial Sequence
  HSA S342H
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYHVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 127 - protein, 585 aa, Artificial Sequence
  HSA S342I
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYIVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 128 - protein, 585 aa, Artificial Sequence
  HSA S342L
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYLVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 129 - protein, 585 aa, Artificial Sequence
  HSA S342N
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYNVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 130 - protein, 585 aa, Artificial Sequence
  HSA S342P
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYPVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 131 - protein, 585 aa, Artificial Sequence
  HSA S342Q
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYQVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 132 - protein, 585 aa, Artificial Sequence
  HSA S342V
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYVVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 133 - protein, 585 aa, Artificial Sequence
  HSA S345H
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVHLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 134 - protein, 585 aa, Artificial Sequence
  HSA S345I
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVILLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 135 - protein, 585 aa, Artificial Sequence
  HSA S345Q
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVQLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 136 - protein, 585 aa, Artificial Sequence
  HSA S349K
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRKAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 137 - protein, 585 aa, Artificial Sequence
  HSA S349Q
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRQAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

(19) United States
(12) Patent Application Publication
(10) Pub. No.: US 2016/0222087 A1
(43) Pub. Date: Aug. 4, 2016

(54) ALBUMIN VARIANTS

(71) Applicant: NOVOZYMES BIOPHARMA DK A/S, Bagsvaerd (DK)

(72) Inventors: Karen DELAHAY, Nottingham (GB); Jason CAMERON, Nottingham (GB)

(73) Assignee: Novozymes Biopharma DK A/S, Bagsvaerd (DK)

(21) Appl. No.: 15/021,602

(22) PCT Filed: Sep. 12, 2014
(86) PCT No.: PCT/EP2014/069561

§ 371 Date: Mar. 11, 2016

(30) Foreign Application Priority Data
Sep. 13, 2013 (EP) 13184465.6

(51) Int. Cl. C07K 14/765

(52) U.S. Cl. CPC C07K 14/765; C07K 2319/31; A61K 38/00

(57) ABSTRACT

The present invention relates to variants of a parent albumin; the variants comprise one or more mutations in Domain II of albumin which lead to a change in binding affinity to FcRn and/or a change in half-life compared to the “parent” albumin. The invention allows tailoring of binding affinity and/or half-life of an albumin to the requirements and desires of a user or application. The invention also relates to fragments and fusion polypeptide of the variant albumins, as well as to polynucleotides encoding the variants, nucleic acid constructs, vectors, host cells comprising the polynucleotides and methods of using said variants.

### REFERENCE TO A SEQUENCE LISTING

This application contains a Sequence Listing in computer readable form, which is incorporated herein by reference.

### BACKGROUND OF THE INVENTION

1. Field of the Invention

The invention relates to variants of albumin or fragments thereof or fusion polypeptides comprising variant albumin or fragments thereof comprising one or more (several) mutations in Domain II of albumin and having a change in binding affinity to FcRn and/or a change in half-life compared to the “parent” albumin, fragment thereof or fusion polypeptide comprising albumin or a fragment thereof. The invention allows tailoring of binding affinity and/or half-life of an albumin to the requirements and desires of a user or application.

2. Description of the Related Art

Albumin is a protein naturally found in the blood plasma of mammals where it is the most abundant protein. It has important roles in maintaining the desired osmotic pressure of the blood and also in transport of various substances in the blood stream. Albumins have been characterized from many species including human, pig, mouse, rat, rabbit and goat and they share a high degree of sequence and structural homology.

Albumin binds in vivo to its receptor, the neonatal Fc receptor (FcRn) “Brambell” and this interaction is known to be important for the plasma half-life of albumin. FcRn is a membrane bound protein, expressed in many cell and tissue types. FcRn has been found to salvage albumin from intracellular degradation (Roopenian D. C. and Akilesh, S. (2007), Nat. Rev. Immunol 7, 715-725.). FcRn is a bifunctional molecule that contributes to maintaining a high level of IgGs and albumin in serum in mammals such as human beings.

Whilst the FcRn-immunoglobulin (IgG) interaction has been characterized in the prior art, the FcRn-albumin interaction is less well characterized. The major FcRn binding site in albumin is localized within Domain III (DIII, 381-585), (Andersen et al. (2010), Clinical Biochemistry 43, 367-372). A number of key amino acids have been shown to be important in binding, notably histidines H464, H510 and H536 and Lys500 (Andersen et al. (2010), Nat. Commun. 3:610. DOI:10.1038/ncomms1607). Data indicates that IgG and albumin bind non-cooperatively to distinct sites on FcRn (Andersen et al. (2006), Eur. J. Immunol 36, 3044-3051; Chaudhury et al. (2006), Biochemistry 45, 4983-4990.).

It is known that mouse FcRn binds IgG from mice and humans whereas human FcRn appears to be more discriminating (Ober et al. (2001) Int. Immunol 13, 1551-1559). Andersen et al. (2010) Journal of Biological Chemistry 285(7):4826-36, describes the affinity of human and mouse FcRn for each of mouse and human albumin (all possible combinations). No binding of albumin from either species was observed at physiological pH to either receptor. At acidic pH, a 100-fold difference in binding affinity was observed. In all cases, binding of albumin and IgG from either species to both receptors were additive.

Human serum albumin (HSA) has been well characterized as a polypeptide of 585 amino acids, the sequence of which can be found in Peters, T., Jr. (1996) All about Albumin: Biochemistry, Genetics and Medical, Applications pp 10, Academic Press, Inc., Orlando (ISBN 0-12-552110-3). It has a characteristic binding to its receptor FcRn, where it binds at pH 6.0 but not at pH 7.4.

The plasma half-life of HSA has been found to be approximately 19 days. A natural variant having lower plasma half-life has been identified (Peach, R. J. and Brennan, S. 0., (1991) Biochim Biophys Acta. 1097:49-54) having the substitution D494N. This substitution generated an N-glycosylation site in this variant, which is not present in the wild-type albumin. It is not known whether the glycosylation or the amino acid change is responsible for the change in plasma half-life.

Albumin has a long plasma half-life and because of this property it has been suggested for use in drug delivery. Albumin has been conjugated to pharmaceutically beneficial compounds (WO2000/69902), and it was found that the conjugate maintained the long plasma half-life of albumin. The resulting plasma half-life of the conjugate was generally considerably longer than the plasma half-life of the beneficial therapeutic compound alone.

Further, albumin has been genetically fused to therapeutically beneficial peptides (WO 2001/79271 A and WO2003/59934) with the typical result that the fusion has the activity of the therapeutically beneficial peptide and a considerably longer plasma half-life than the plasma half-life of the therapeutically beneficial peptides alone.

Otagiri et al. (2009), Biol. Pharm. Bull. 32(4), 527-534, discloses more than 70 albumin variants. Of these variants, 24 positions in Domain II are found to be mutated e.g. E382K. A natural variant lacking the last 175 amino acids at the carboxy terminus has been shown to have reduced half-life (Andersen et al. (2010), Clinical Biochemistry 43, 367-372). Iwao et al. (2007) studied the half-life of naturally occurring human albumin variants using a mouse model, and found that K541E and K560E had reduced half-life, E501K and E570K had increased half-life and K573E had almost no effect on half-life (Iwao, et. al. (2007) B.B.A. Proteins and Proteomics 1774, 1582-1590). Minchiotti et al. (2008) Human Mutation 29(8), 1007-1016, discloses several natural variants.

WO2011/051489 and WO2012/150319 disclose a number of point mutations in albumin which modulate the binding of albumin to FcRn, WO2010/092135 discloses a number of point mutations in albumin which increase the number of thiols available for conjugation in the albumin, the disclosure is silent about the effect of the mutations on the binding of the albumin to FcRn. WO2011/103076 discloses albumin variants, each containing a substitution in Domain III of HSA. WO2012/112188 discloses albumin variants containing substitutions in Domain III of HSA. WO2013/075066 discloses albumin variants which modulate the binding affinity of albumin to FcRn.

Albumin has the ability to bind a number of ligands and these become associated (associates) with albumin thereby forming ‘associates’. This property has been utilized to extend the plasma half-life of drugs having the ability to non-covalently bind to albumin. This can also be achieved by binding a pharmaceutical beneficial compound, which has little or no albumin binding properties, to a moiety having albumin binding properties, see review article and reference therein, Kratz (2008) Journal of Controlled Release 132, 171-183.

Albumin is used in preparations of pharmaceutically beneficial compounds, in which such a preparation may be for example, but not limited to, a nanoparticle or microparticle of albumin. In these examples the delivery of a pharmaceutically beneficial compound or mixture of compounds may benefit from alteration in the albumin's affinity to its receptor where the beneficial compound has been shown to associate with albumin for the means of delivery. It is not clear what determines the plasma half-life of the formed associates (for example but not limited to Levemir®, Kurtzhals P et al. Biochem. J. 1995; 312:725-731), conjugates or fusion polypeptides but it appears to be a result of the combination of the albumin and the selected pharmaceutically beneficial compound/polypeptide. It would be desirable to be able to control the plasma half-life of given albumin conjugates, associates or albumin fusion polypeptides so that a longer or shorter plasma half-life can be achieved than given by the components of the association, conjugation or fusion, in order to be able to design a particular drug according to the particulars of the indication intended to be treated.

Albumin is known to accumulate and be catabolized in tumors, it has also been shown to accumulate in inflamed joints of rheumatoid arthritis sufferers. See review article and reference therein, Kratz (2008) Journal of Controlled Release 132, 171-183. It is envisaged that HSA variants with increased affinity for FcRn would be advantageous for the delivery of pharmaceutically beneficial compounds.

It may even be desirable to have variants of albumin that have little or no binding to FcRn in order to provide shorter half-lives or controlled serum pharmacokinetics as described by Kenanova et al. (2009) J. Nucl. Med.; 50 (Supplement 2):1582).

Kenanova et al. (2010, Protein Engineering, Design & Selection 23(10): 789-798; WO2010/118169) discloses a docking model comprising a structural model of Domain III of HSA (solved at pH 7 to 8) and a structural model of FcRn (solved at pH 6.4). Kenanova et al. discloses that positions 464, 505, 510, 531 and 535 in Domain III potentially interact with FcRn. The histidines at positions 464, 510 and 535 were identified as being of particular interest by Chaudhury et al., (2006, op. cit.) and these were shown to have a significant reduction in affinity and shorter half-life in mouse by Kenanova (2010, op. cit.). However, the studies of Kenanova et al. are limited to Domain III of HSA and therefore do not consider HSA in its native intact configuration. Furthermore, the identified positions result in a decrease in affinity for the FcRn receptor.

The present invention surprisingly identifies that alterations to amino acids which are not located in the principle albumin-receptor binding face can affect the binding affinity of albumin to the receptor. Domains I and III make up the binding face of albumin that interacts with the FcRn receptor. Therefore, the invention provides further variants having altered binding affinity to the FcRn receptor wherein the variants comprise one or more (several) alterations in Domain II of albumin, particularly from positions corresponding to 195 to 384 of SEQ ID NO: 2. The albumin moiety or moieties may therefore be used to tailor the binding affinity to FcRn and/or half-life of fusion polypeptides, conjugates, associates, nanoparticles and compositions comprising the albumin moiety. Since Domain II of albumin does not include surface-exposed residues involved in the binding interface, it is surprising that alterations in Domain II affect the binding affinity of albumin to FcRn. For example, Oganesyan et al ((2014) Journal of Biological Chemistry 289(11):7812-24) observed no direct contribution from HSA DII to the HSA-FcRn complex.

### SUMMARY OF THE INVENTION

The present invention relates to albumin variants comprising one or more (several) alterations at positions in Domain II of albumin particularly from positions corresponding to 195 to 384 of SEQ ID NO: 2. More particularly, the invention relates to albumin variants comprising one or more (several) alterations at positions corresponding to positions comprising or selected from the group consisting of 198, 206, 340, 341, 342, 343, 344, 345, 348, 349, 352, 381, 382, 383, and/or 384, of the mature polypeptide of SEQ ID NO: 2 or equivalent positions of other albumins or fragments thereof. Positions 342 to 384 form helices IIB-h3 and IIb-h4 in the structure of albumin.

The present invention also relates to isolated polynucleotides encoding the variants; nucleic acid constructs, vectors, and host cells comprising the polynucleotides; and methods of producing the variants.

The invention also relates to conjugates or associates comprising the variant albumin or fragment thereof according to the invention and a beneficial therapeutic moiety or to a fusion polypeptide comprising a variant albumin or fragment thereof of the invention and a fusion partner polypeptide.

The invention further relates to compositions comprising the variant albumin, fragment thereof, fusion polypeptide comprising variant albumin or fragment thereof or conjugates comprising the variant albumin or fragment thereof, according to the invention or associates comprising the variant albumin or fragment thereof, according to the invention. The compositions are preferably pharmaceutical compositions.

The invention further relates to a pharmaceutical composition comprising a variant albumin, fragment thereof, fusion polypeptide comprising variant albumin or fragment thereof or conjugates comprising the variant albumin or fragment thereof, or associates comprising the variant albumin or fragment thereof.

The invention also relates to the use of the variants, fragments, fusion polypeptides, conjugates, associates, nanoparticles and microparticles.

The invention also relates to a method for preparing a variant albumin, fragment thereof, fusion polypeptide comprising variant albumin or fragment thereof or conjugates comprising the variant albumin or fragment thereof, or associates comprising the variant albumin or fragment thereof.

### BRIEF DESCRIPTION OF THE FIGURES

FIG. 1: Multiple alignment of amino acid sequences of (i) full length mature HSA (Hu_1_2_3), (ii) an albumin variant comprising Domain I and Domain III of HSA (Hu_1_3), (iii) an albumin variant comprising Domain II and Domain III of HSA (Hu_2_3), (iv) full-length Macaca mulatta albumin (Mac_mul), (v) full-length Rattus norvegicus albumin (Rat) and (vi) full-length Mus musculus albumin (Mouse). Positions 500, 550 and 573 (relative to full length HSA) are indicated by arrows. In FIG. 1, Domains I, II and III are referred to as 1, 2 and 3 (respectively).

FIG. 2: Multiple alignment of amino acid sequences of mature albumin from human, sheep, mouse, rabbit and goat and immature albumins from chimpanzee (“Chimp”), macaque, hamster, guinea pig, rat, cow, horse, donkey, dog, chicken, and pig. The Start and End amino acids of domains 1, 2 and 3 (as defined by Dockal et al. (The Journal of Biological Chemistry, 1999, Vol. 274(41): 29303-29310)) are indicated with respect to mature human albumin.

FIG. 3: Conserved groups of amino acids based on their properties.

FIG. 4: Representation of shFcRn-HSA docking model. (A-B) Two orientations of the complex are shown. Albumin is shown by a space-filling diagram, FcRn is shown as a ribbon diagram. The core binding interface of HSA is highlighted in pink (in grey-scale this is seen as the darkest (almost black) region; DI (CBI)), while the area distally localized from the interface is shown as DII (orange) and DIII is split into sub-domains DIIIa (in colour, this is cyan) and DIIIb (in colour, this is blue).

FIG. 5: Domain structure of HSA (1E78.pdb from RCSB Protein Databank; Bhattacharya et al. (2000), Binding of the general anesthetics propofol and halothane to human serum albumin. High resolution crystal structures. J. Biol. Chem. 275: 38731)).

FIG. 6: SPR sensorgrams showing the binding affinity of WT HSA and variants comprising combinations of domains injected over immobilized shFcRn at pH 6.0. (Andersen et al., 2012). WT: wild-type, DIII: Domain III, DI-DIII: Domain I-Domain III, DII-DIII: Domain II-Domain III; DI-DIII: Domain I-Domain III).

FIG. 7: Superposition of Domain I (residues 1-194 of SEQ ID NO: 2) onto Domain II (residues 195-380 of SEQ ID NO: 2). Domain I is shown in dark grey and DII in light grey. Sub-domains are labelled A and B.

FIG. 8: Alignment of Domain I (residues 16-194 of SEQ ID NO: 2) and Domain II (residues 208-384 of SEQ ID NO: 2) anchored using disulphide bonds. Residues 1-15 and 195-207 are not included due to limited structural homology in these regions. Boxes indicate strictly conserved residues. Dotted line indicates pairwise comparison variants depicted in FIG. 9 (R348F and L349F). Dashed line indicates conserved acidic residues used for pairwise comparison variants at positions 377-384.

FIG. 9: Example of pairwise comparison variants, relevant residues are shown in stick form. DI is shown in dark grey and DII in light grey. A. R348F B. R349F.

FIG. 10: Domain I/II boundary. Domain I (dark gray) superimposed on Domain II in full length HSA (light gray). K195, L198 and F206 are shown in stick representation. K195 represents the DI/DII boundary. S5 (representing the N-terminus) and Domain III are labelled.

FIG. 11: Domain II/III boundary. Domain I (dark gray) superimposed on Domain II in full length HSA (light gray). Spheres represent the residues involved in the junction of the DI-DIII variant (A194 and V381). B. The Domain II/III spanning helix is kinked (dotted lines).

FIG. 12: Position of variants resulting in altered FcRn binding (indicated by spheres). Domains and positions L198, F206, S342, L349, V381 and P384 (residue defining the domain boundary) are labeled.

### DEFINITIONS

Variant: The term “variant” means a polypeptide derived from a parent albumin by one or more (several) alteration(s), i.e. a substitution, insertion, and/or deletion, at one or more (several) positions. A substitution means a replacement of an amino acid occupying a position with a different amino acid; a deletion means removal of an amino acid occupying a position; and an insertion means adding 1 or more, such as 1, 2, 3, 4, 5, 6, 7, 8, 9 or 10, preferably 1-3 amino acids immediately adjacent an amino acid occupying a position. In relation to substitutions, ‘immediately adjacent’ may be to the N-side (‘upstream’) or C-side (‘downstream’) of the amino acid occupying a position (‘the named amino acid’). Therefore, for an amino acid named/numbered ‘X’, the insertion may be at position ‘X+1’ (‘downstream’) or at position ‘X−1’ (‘upstream’).

Mutant: The term “mutant” means a polynucleotide encoding a variant.

Wild-Type Albumin: The term “wild-type” (WT) albumin means albumin having the same amino acid sequence as naturally found in an animal or in a human being.

Parent Albumin: The term “parent” or “parent albumin” means an albumin to which an alteration is made by the hand of man to produce the albumin variants of the invention. The parent may be a naturally occurring (wild-type) polypeptide or an allele thereof, or even a variant thereof.

Albumin: Albumins are proteins and constitute the most abundant protein in plasma in mammals and albumins from a long number of mammals have been characterized by biochemical methods and/or by sequence information. Several albumins, e.g, human serum albumin (HSA), have also been characterized crystallographically and the structure determined (HSA: He X M, Carter D C (July 1992). “Atomic structure and chemistry of human serum albumin”. Nature 358 (6383): 209-15; horse albumin: Ho, J. X. et al. (2001). X-ray and primary structure of horse serum albumin (Equus caballus) at 0.27-nm resolution. Eur J Biochem. 215(1):205-12).

The term “albumin” means a protein having the same and/or very similar three dimensional (tertiary) structure as HSA or HSA domains and having similar properties to HSA or to the relevant domains. Similar three dimensional structures are for example the structures of the albumins from the species mentioned herein. Some of the major properties of albumin are i) its ability to regulate plasma volume (oncotic activity), ii) a long plasma half-life of around 19 days±5 days, iii) binding to FcRn, iv) ligand-binding, e.g. binding of endogenous molecules such as acidic, lipophilic compounds including bilirubin, fatty acids, hemin and thyroxine (see also Table 1 of Kragh-Hansen et al., 2002, Biol. Pharm. Bull. 25, 695, hereby incorporated by reference), v) binding of small organic compounds with acidic or electronegative features e.g. drugs such as warfarin, diazepam, ibuprofen and paclitaxel (see also Table 1 of Kragh-Hansen et al., 2002, Biol. Pharm. Bull. 25, 695, hereby incorporated by reference). Not all of these properties need to be fulfilled in order to characterize a protein or fragment as an albumin. If a fragment, for example, does not comprise a domain responsible for binding of certain ligands or organic compounds the variant of such a fragment will not be expected to have these properties either.

Albumins have generally a long plasma half-life of approximately 20 days or longer, e.g, HSA has a plasma half-life of 19 days. It is known that the long plasma half-life of HSA is mediated via interaction with its receptor FcRn, however, an understanding or knowledge of the exact mechanism behind the long half-life of HSA is not essential for the invention.

As examples of albumin proteins as starting parent “backbones” for making albumin variants according to the invention can be mentioned human serum albumin (e.g. AAA98797 or P02768-1, SEQ ID NO: 2 (mature), SEQ ID NO: 4 (immature)), primate serum albumin, (such as chimpanzee serum albumin (e.g. predicted sequence XP_517233.2 SEQ ID NO: 5), gorilla serum albumin or macaque serum albumin (e.g. NP_001182578, SEQ ID NO: 6), rodent serum albumin (such as hamster serum albumin (e.g. A6YF56, SEQ ID NO: 7), guinea pig serum albumin (e.g. Q6WDN9-1, SEQ ID NO: 8), mouse serum albumin (e.g. AAH49971 or P07724-1 Version 3, SEQ ID NO: 9) and rat serum albumin (e.g. AAH85359 or P02770-1 Version 2, SEQ ID NO: 10))), bovine serum albumin (e.g. cow serum albumin P02769-1, SEQ ID NO: 11), equine serum albumin such as horse serum albumin (e.g. P35747-1, SEQ ID NO: 12) or donkey serum albumin (e.g. Q5XLE4-1, SEQ ID NO: 13), rabbit serum albumin (e.g. P49065-1 Version 2, SEQ ID NO: 14), goat serum albumin (e.g. ACF10391, SEQ ID NO: 15), sheep serum albumin (e.g. P14639-1, SEQ ID NO: 16), dog serum albumin (e.g. P49822-1, SEQ ID NO: 17), chicken serum albumin (e.g. P19121-1 Version 2, SEQ ID NO: 18) and pig serum albumin (e.g. P08835-1 Version 2, SEQ ID NO: 19) or a polypeptide having at least 70, 75, 80, 85, 90, 91, 92, 93, 94, 95, 96, 97, 98 or at least 99% amino acid identity to such an albumin. The parent or reference albumin may be an artificial variant such as HSA K573P (SEQ ID NO: 3) or a chimeric albumin such as the N-terminal of HSA and the C-terminal of macaca albumin (SEQ ID NO: 20), N-terminal of HSA and the C-terminal of mouse albumin (SEQ ID NO: 21), N-terminal of HSA and the C-terminal of rabbit albumin (SEQ ID NO: 22), N-terminal of HSA and the C-terminal of sheep albumin (SEQ ID NO: 23).

Other examples of albumin, which are also included in the scope of this application, include ovalbumin (e.g. P01012.pro: chicken ovalbumin; 073860.pro: turkey ovalbumin).

HSA as disclosed in SEQ ID NO: 2 or any naturally occurring allele thereof, is the preferred parent albumin according to the invention. HSA is a protein consisting of 585 amino acid residues and has a molecular weight of 67 kDa. In its natural form it is not glycosylated. The skilled person will appreciate that natural alleles may exist having essentially the same properties as HSA but having one or more (several) amino acid changes compared to SEQ ID NO: 2, and the inventors also contemplate the use of such natural alleles as parent albumins according to the invention.

The parent albumin, a fragment thereof, or albumin part of a fusion polypeptide comprising albumin or a fragment thereof according to the invention preferably has a sequence identity to the sequence of HSA shown in SEQ ID NO: 2 of at least 60%, preferably at least 70%, preferably at least 80%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, more preferred at least 96%, more preferred at least 97%, more preferred at least 98% and most preferred at least 99%, at least 99.2%, at least 99.4%, at least 99.6% or at least 99.8% or 100%. It is preferred that the parent albumin maintains at least one of the major properties of albumin or a similar tertiary structure as an albumin, such as HSA. The sequence identity may be over the full-length of SEQ ID NO: 2 or over a molecule consisting or comprising of a fragment such as one or more (several) domains of SEQ ID NO: 2 such as a molecule consisting of or comprising Domain III (e.g. SEQ ID NO: 27), a molecule consisting of or comprising Domain II and Domain III (e.g. SEQ ID NO: 25), a molecule consisting of or comprising Domain I and Domain III (e.g. SEQ ID NO: 24), a molecule consisting of or comprising two copies of Domain III (e.g. SEQ ID NO: 26), a molecule consisting of or comprising three copies of Domain III (e.g. SEQ ID NO: 28) or a molecule consisting of or comprising Domain I and two copies of Domain III (e.g. SEQ ID NO: 29).

The parent preferably comprises or consists of the amino acid sequence of SEQ ID NO: 4 (immature sequence of HSA) or SEQ ID NO: 2 (mature sequence of HSA).

In another embodiment, the parent is an allelic variant of the mature polypeptide of SEQ ID NO: 2.

The parent albumin may be encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 1, or (ii) the full-length complementary strand of (i) (J. Sambrook, E. F. Fritsch, and T. Maniatis, 1989, Molecular Cloning, A Laboratory Manual, 2d edition, Cold Spring Harbor, New York).

The polynucleotide of SEQ ID NO: 1 or a subsequence thereof, as well as the amino acid sequence of SEQ ID NO: 2 or a fragment thereof, may be used to design nucleic acid probes to identify and clone DNA encoding a parent from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 14, e.g, at least 25, at least 35, or at least 70 nucleotides in length. Preferably, the nucleic acid probe is at least 100 nucleotides in length, e.g, at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labelled for detecting the corresponding gene (for example, with ³²P, ³H, ³⁵S, biotin, or avidin). Such probes are encompassed by the invention.

A genomic DNA or cDNA library prepared from such other organisms may be screened for DNA that hybridizes with the probes described above and encodes a parent. Genomic or other DNA from such other organisms may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that is homologous with SEQ ID NO: 1 or a subsequence thereof, the carrier material is used in a Southern blot.

For purposes of the invention, hybridization indicates that the polynucleotide hybridizes to a labelled nucleotide probe corresponding to the polynucleotide shown in SEQ ID NO: 1, its complementary strand, or a subsequence thereof, under low to very high stringency conditions. Molecules to which the probe hybridizes can be detected using, for example, X-ray film or any other detection means known in the art.

The nucleic acid probe may comprise or consist of the mature polypeptide coding sequence of SEQ ID NO: 1, i.e. nucleotides 1 to 1785 of SEQ ID NO: 1. The nucleic acid probe may comprise or consist of a polynucleotide that encodes the polypeptide of SEQ ID NO: 2 or a fragment thereof.

For long probes of at least 100 nucleotides in length, very low to very high stringency conditions are defined as pre-hybridization and hybridization at 42° C. in 5×SSPE, 0.3% SDS, 200 micrograms/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally. The carrier material is finally washed three times each for 15 minutes using 2×SSC, 0.2% SDS at 45° C. (very low stringency), 50° C. (low stringency), 55° C. (medium stringency), 60° C. (medium-high stringency), 65° C. (high stringency), or 70° C. (very high stringency).

For short probes that are about 15 nucleotides to about 70 nucleotides in length, stringency conditions are defined as pre-hybridization and hybridization at about 5° C. to about 10° C. below the calculated Tₘ using the calculation according to Bolton and McCarthy (1962, Proc. Natl. Acad. Sci. USA 48: 1390) in 0.9 M NaCl, 0.09 M Tris-HCl pH 7.6, 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per ml following standard Southern blotting procedures for 12 to 24 hours optimally. The carrier material is finally washed once in 6×SCC plus 0.1% SDS for 15 minutes and twice each for 15 minutes using 6×SSC at 5° C. to 10° C. below the calculated Tₘ.

The parent may be encoded by a polynucleotide with a sequence identity to the mature polypeptide coding sequence of SEQ ID NO: 1 of at least 60%, e.g, at least 65%, at least 70%, at least 75%, at least 80%, at least 85%, at least 90%, at least 95%, at least 96%, at least 97%, at least 98%, at least 99%, or 100%, which encodes a polypeptide which is able to function as an albumin. In an embodiment, the parent is encoded by a polynucleotide comprising or consisting of SEQ ID NO: 1.

Albumin moiety: The albumin part of a fusion polypeptide, conjugate, associate, nanoparticle or composition comprising the albumin variant or fragment thereof according to the invention, may be referred to as an ‘albumin moiety’ or ‘albumin component’. A polypeptide according to the invention may comprise or consist of an albumin moiety.

FcRn and shFcRn: The term “FcRn” means the neonatal Fc receptor (FcRn), particularly the human neonatal Fc receptor. shFcRn is a soluble recombinant form of FcRn. shFcRn is a heterodimer of SEQ ID NO: 30 (truncated heavy chain of the major histocompatibility complex class I-like Fc receptor (FCGRT)) and SEQ ID NO: 31 (beta-2-microglobulin). Together, SEQ ID NO: 30 and 31 form hFcRn.

Isolated variant: The term “isolated variant” means a variant in a form or environment which does not occur in nature. Non-limiting examples of isolated variants include (1) any non-naturally occurring variant, (2) any variant that is at least partially removed from one or more (several) or all of the naturally occurring constituents with which it is associated in nature; (3) any variant modified by the hand of man relative to the polypeptide from which it is derived (e.g. the polypeptide from which it is derived as found in nature); or (4) any variant modified by increasing the amount of the variant relative to other components with which it is naturally associated (e.g, multiple copies of a gene encoding the substance; use of a stronger promoter than the promoter naturally associated with the gene encoding the substance). An isolated variant may be present in a fermentation broth sample.

Substantially pure variant: The term “substantially pure variant” means a preparation that contains at most 10%, at most 8%, at most 6%, at most 5%, at most 4%, at most 3%, at most 2%, at most 1%, and at most 0.5% by weight of other polypeptide material with which it is natively or recombinantly associated. Preferably, the variant is at least 92% pure, e.g, at least 94% pure, at least 95% pure, at least 96% pure, at least 97% pure, at least 98% pure, at least 99%, at least 99.5% pure, and 100% pure by weight of the total polypeptide material present in the preparation. Purity may be determined by SDS-PAGE or GP-HPLC. The variants of the invention are preferably in a substantially pure form. This can be accomplished, for example, by preparing the variant by well-known recombinant methods and by purification methods.

Mature polypeptide: The term “mature polypeptide” means a polypeptide in its final form following translation and any post-translational modifications, such as N-terminal processing, C-terminal truncation, glycosylation, phosphorylation, etc. The mature polypeptide may be amino acids 1 to 585 of SEQ ID NO: 2, e.g. with the inclusion of alterations according to the invention and/or any post-translational modifications.

Mature polypeptide coding sequence: The term “mature polypeptide coding sequence” means a polynucleotide that encodes a mature albumin polypeptide. The mature polypeptide coding sequence may be nucleotides 1 to 1758 of SEQ ID NO: 1 e.g. with the alterations required to encode a variant according to the invention.

Sequence Identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Fragment: The term “fragment” means a polypeptide having one or more (several) amino acids deleted from the amino and/or carboxyl terminus of an albumin and/or an internal region of albumin that has retained the ability to bind to FcRn. Fragments may consist of one uninterrupted sequence derived from HSA or it may comprise two or more (several) sequences derived from HSA. The fragments according to the invention have a size of more than approximately 20 amino acid residues, preferably more than 30 amino acid residues, more preferred more than 40 amino acid residues, more preferred more than 50 amino acid residues, more preferred more than 75 amino acid residues, more preferred more than 100 amino acid residues, more preferred more than 200 amino acid residues, more preferred more than 300 amino acid residues, even more preferred more than 400 amino acid residues and most preferred more than 500 amino acid residues. A fragment may comprise or consist of one more domains of albumin such as DI+DII, DI+DIII, DII+DIII, DIII+DIII, DI+DIII+DIII, DIII+DIII+DIII, or fragments of such domains or combinations of domains.

Domains I, II and III may be defined with reference to HSA (SEQ ID NO: 2). For example, HSA Domain I may consist of or comprise amino acids 1 to 194 (±1 to 15 amino acids) of SEQ ID NO: 2, HSA Domain II may consist of or comprise amino acids 192 (±1 to 15 amino acids) to 387 (±1 to 15 amino acids) of SEQ ID NO: 2 and Domain III may consist of or comprise amino acid residues 381 (±1 to 15 amino acids) to 585 (±1 to 15 amino acids) of SEQ ID NO: 2. “±1 to 15 amino acids” means that the residue number may deviate by 1, 2, 3, 4, 5, 6, 7, 8, 9, 10, 11, 12, 13, 14, or 15 amino acids to the C-terminus and/or to the N-terminus of the stated amino acid position. Examples of domains I, II and III are described by Dockal et al. (The Journal of Biological Chemistry, 1999, Vol. 274(41): 29303-29310) and Kjeldsen et al. (Protein Expression and Purification, 1998, Vol 13: 163-169) and are tabulated below.

| Amino acid residues of HSA | | |
|---|---|---|
| domains I, II and III with | | |
| reference to SEQ ID NO: 2 | Dockal et al | Kjeldsen et al |
| Domain I | 1 to 197 | 1 to 192 |
| Domain II | 189 to 385 | 193 to 382 |
| Domain III | 381 to 585 | 383 to 585 |

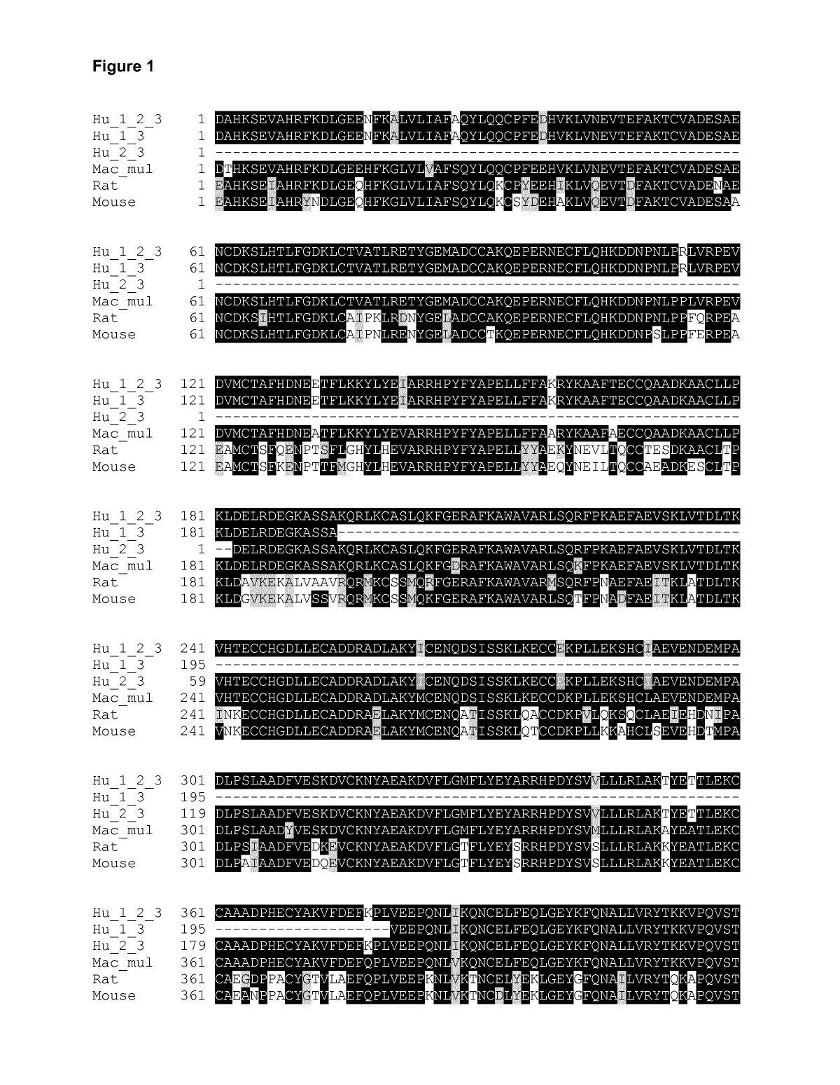

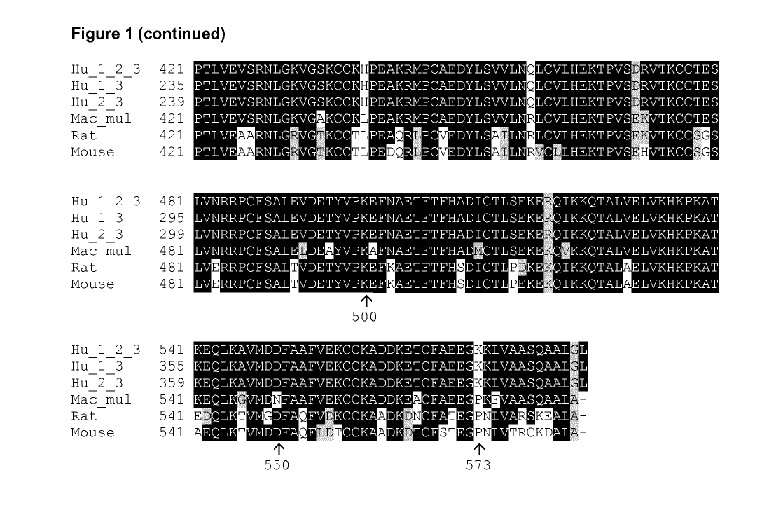

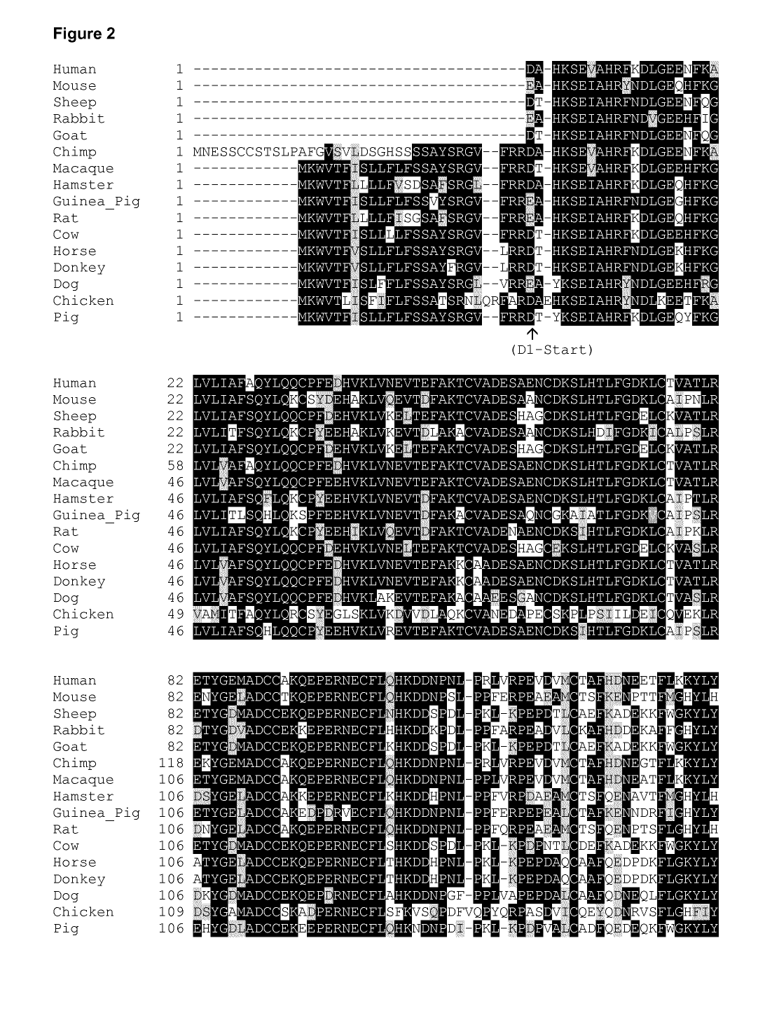

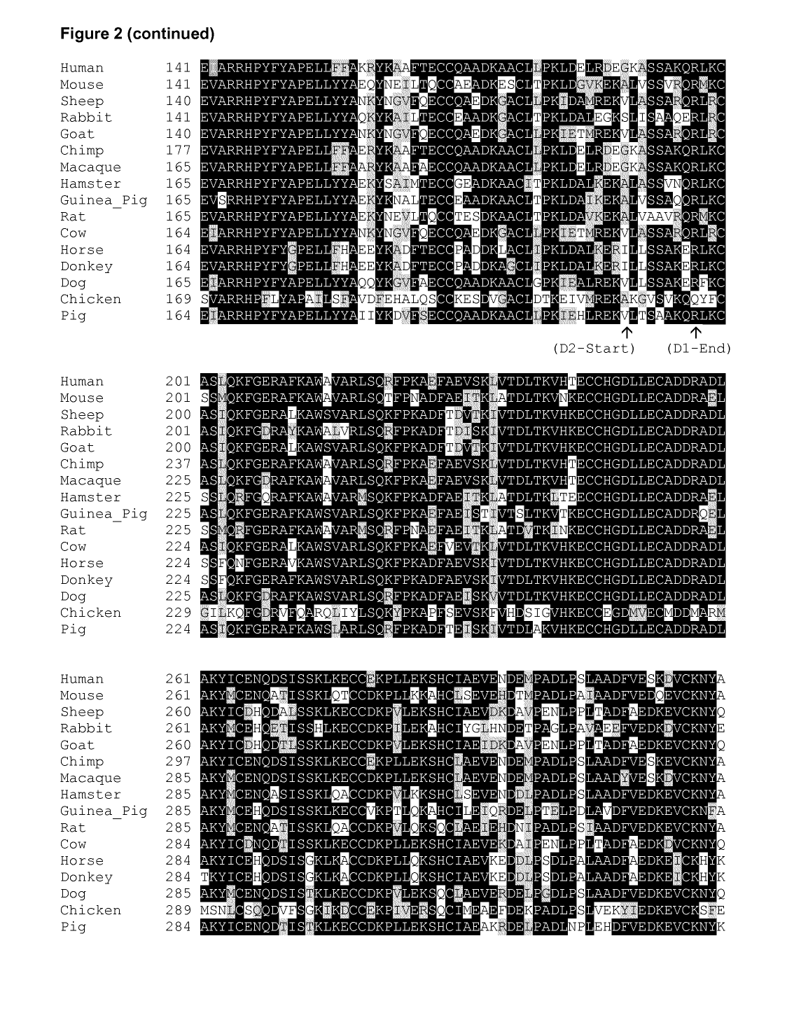

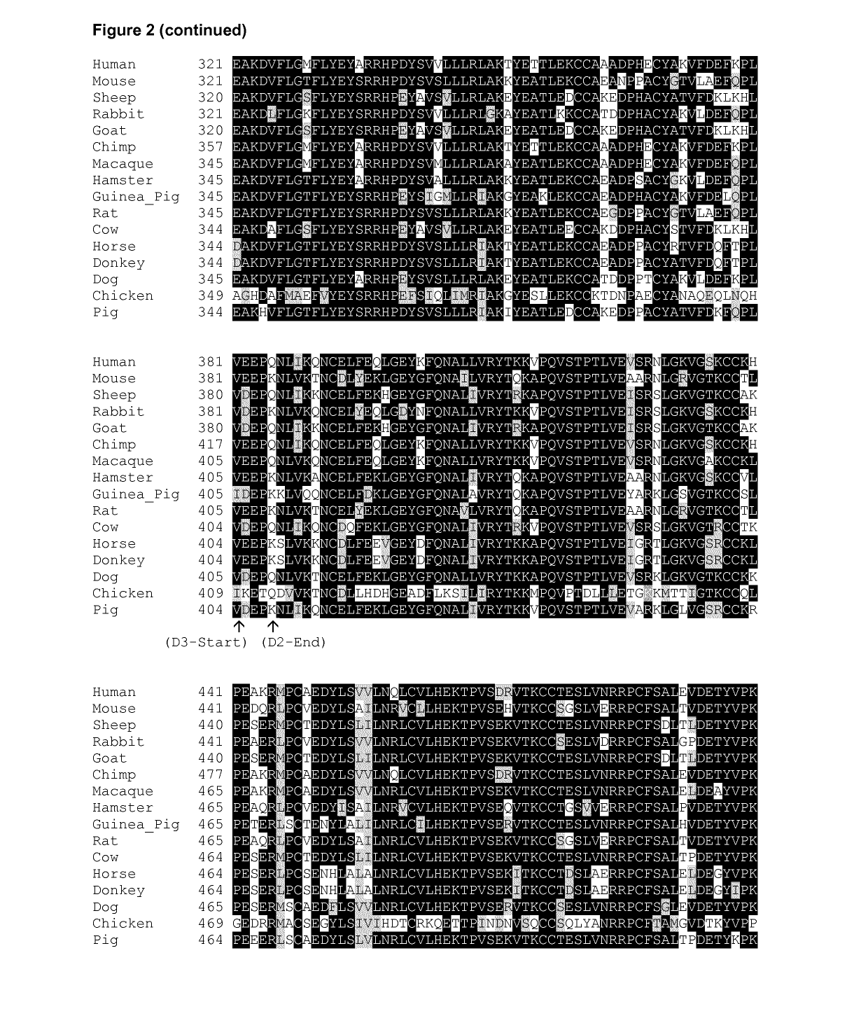

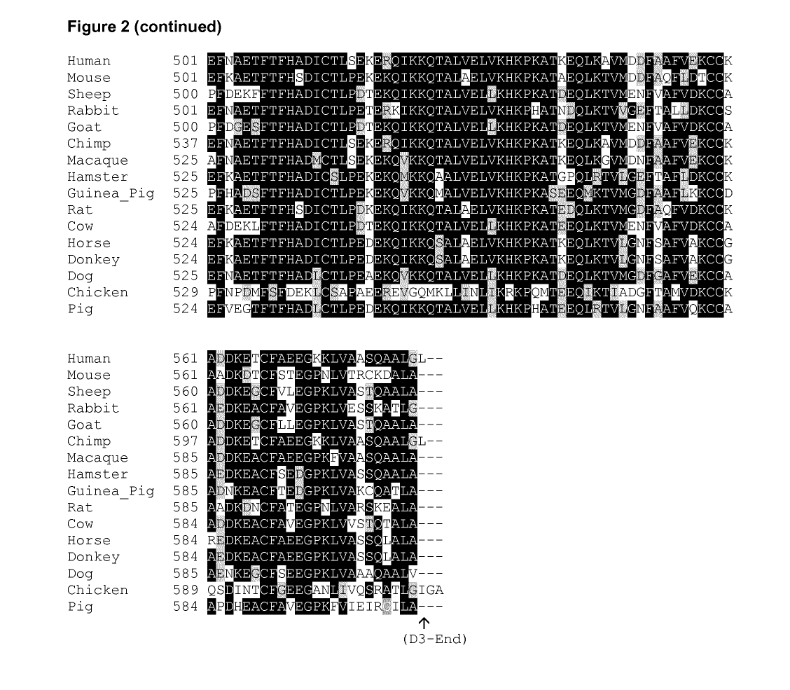

The skilled person can identify domains I, II and III in non-human albumins by amino acid sequence alignment with HSA, for example using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. The optional parameters used are gap open penalty of 10, gap extension penalty of 0.5, and the EBLOSUM62 (EMBOSS version of BLOSUM62) substitution matrix. Other suitable software includes MUSCLE ((Multiple sequence comparison by log-expectation, Robert C. Edgar, Version 3.6, http://www.drive5.com/muscle; Edgar (2004) Nucleic Acids Research 32(5), 1792-97 and Edgar (2004) BMC Bioinformatics, 5(1):113) which may be used with the default settings as described in the User Guide (Version 3.6, September 2005). Versions of MUSCLE later than 3.6 may also be used for any aspect of the invention). Examples of suitable alignments are provided in FIGS. 1 and 2.

It is preferred that domains have at least 70, 75, 80, 85, 90, 95, 96, 97, 98, 99, 99.5% identity or 100% identity to Domain I, II or III of HSA (SEQ ID NO: 2).

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Coding sequence: The term “coding sequence” means a polynucleotide, which directly specifies the amino acid sequence of its translated polypeptide product. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, cDNA, synthetic, or recombinant polynucleotide.

cDNA: The term “cDNA” means a DNA molecule that can be prepared by reverse transcription from a mature, spliced, mRNA molecule obtained from a eukaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the invention.

Control sequences: The term “control sequences” means all nucleic acid sequences necessary for the expression of a polynucleotide encoding a variant of the invention. Each control sequence may be native (i.e. from the same gene) or foreign (i.e. from a different gene) to the polynucleotide encoding the variant or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant.

Operably linked: The term “operably linked” means a configuration in which a control sequence is placed at an appropriate position relative to the coding sequence of a polynucleotide such that the control sequence directs the expression of the coding sequence.

Expression: The term “expression” includes any step involved in the production of the variant including, but not limited to, transcription, post-transcriptional modification, translation, post-translational modification, and secretion.

Expression vector: The term “expression vector” means a linear or circular DNA molecule that comprises a polynucleotide encoding a variant and is operably linked to control sequences that provide for its expression.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Plasma half-life: Plasma half-life is ideally determined in vivo in suitable individuals. However, since it is time consuming and expensive and inevitably there are ethical concerns connected with doing experiments in animals or man, it is desirable to use an in vitro assay for determining whether plasma half-life is extended or reduced. It is known that the binding of albumin to its receptor (FcRn) is important for plasma half-life and the correlation between receptor binding and plasma half-life is that a higher affinity of albumin to its receptor leads to longer plasma half-life. Thus for the invention a higher affinity of albumin to FcRn is considered indicative of an increased plasma half-life and a lower affinity of albumin to its receptor is considered indicative of a reduced plasma half-life.

In this application and claims the binding of albumin to its receptor FcRn is described using the term affinity and the expressions “stronger” or “weaker”. Thus, it should be understood that a molecule having a higher affinity to FcRn than HSA is considered to bind stronger to FcRn than HSA and a molecule having a lower affinity to FcRn than HSA is considered to bind weaker to FcRn than HSA. The term ‘binding coefficient’ can be used instead of the term ‘binding affinity’.

The terms “longer plasma half-life” or “shorter plasma half-life” and similar expressions are understood to be in relationship to the corresponding parent or reference or corresponding albumin molecule. Thus, a longer plasma half-life with respect to a variant albumin of the invention means that the variant has longer plasma half-life than the corresponding albumin having the same sequences except for the alteration(s) described herein, e.g. at one or more (several) positions corresponding to positions comprising or selected from the group consisting of 198, 206, 340, 341, 342, 343, 344, 345, 348, 349, 352, 381, 382, 383, and/or 384, of HSA (SEQ ID NO: 2).

Reference: a reference is an albumin, fusion, conjugate, composition, associate or nanoparticle to which an albumin variant, fusion, conjugate, composition, associate or nanoparticle is compared. The reference may comprise or consist of full length albumin (such as HSA or a natural allele thereof) or a fragment thereof. A reference may also be referred to as a ‘corresponding’ albumin, fusion, conjugate, composition, associate or nanoparticle to which an albumin variant, fusion, conjugate, composition, associate or nanoparticle is compared. A reference may comprise or consist of HSA (SEQ ID NO: 2) or a fragment, fusion, conjugate, associate, nanoparticle or microparticle thereof. Preferably, the reference is identical to the polypeptide, fusion polypeptide, conjugate, composition, associate, nanoparticle or microparticle according to the invention (“being studied”) with the exception of the albumin moiety. Preferably the albumin moiety of the reference comprises or consists of an albumin (e.g. HSA, SEQ ID NO: 2) or a fragment thereof. The amino acid sequence of the albumin moiety of the reference may be longer than, shorter than or, preferably, the same (±1 to 15 amino acids) length as the amino sequence of the albumin moiety of the polypeptide, fusion polypeptide, conjugate, composition, associate, nanoparticle or microparticle according to the invention (“being studied”).

Equivalent amino acid positions: Throughout this specification amino acid positions are defined in relation to full-length mature human serum albumin (i.e. without leader sequence, SEQ ID NO: 2). However, the skilled person understands that the invention also relates to variants of non-human albumins (e.g. those disclosed herein) and/or fragments of a human or non-human albumin. Equivalent positions can be identified in fragments of human serum albumin, in animal albumins and in fragments, fusions and other derivative or variants thereof by comparing amino acid sequences using pairwise (e.g. ClustalW) or multiple (e.g. MUSCLE) alignments. For example, FIG. 1 shows that positions equivalent to 500, 550 and 573 in full length human serum albumin are easily identified in fragments of human serum albumin and in albumins of other species. Positions 500, 550 and 573 are indicated by arrows. Further details are provided in Table 1 below.

TABLE 1
Example of identification of equivalent positions
in HSA, animal albumins and albumin fragments

| | Albumin | | | Position equivalent to human | | |
|---|---|---|---|---|---|---|
| Organism (accession | Full length | Fragment | Total length of | serum albumin (native amino acid): | | |
| number of protein) | or fragment | details | mature protein | 500 (K) | 550 (D) | 573 (K) |
| Homo sapiens | Full length | — | 585 | 500 (K) | 550 (D) | 573 (K) |
| (AAA98797) | | | | | | |
| Homo sapiens | Fragment | DI, DIII | 399 | 314 (K) | 364 (D) | 387 (K) |
| Homo sapiens | Fragment | DI, DIII | 403 | 318 (K) | 368 (D) | 391 (K) |
| Macaca mulatta | Full length | — | 584 | 500 (K) | 550 (N) | 573 (P) |
| (NP_001182578) | | | | | | |
| Rattus norvegicus | Full length | — | 584 | 500 (K) | 550 (D) | 573 (P) |
| (AAH85359) | | | | | | |
| Mus musculus | Full length | — | 584 | 500 (K) | 550 (D) | 573 (P) |
| (AAH49971) | | | | | | |

FIG. 1 was generated by MUSCLE using the default parameters including output in ClustalW 1.81 format. The raw output data was shaded using BoxShade 3.21 (http://www.ch.embnet.org/software/BOX_form.html) using Output Format: RTF_new; Font Size: 10; Consensus Line: no consensus line; Fraction of sequences (that must agree for shading): 0.5; Input sequence format: ALN. Therefore, throughout this specification amino acid positions defined in human serum albumin also apply to equivalent positions in fragments, derivatives or variants and fusions of human serum albumin, animals from other species and fragments and fusions thereof. Such equivalent positions may have (i) a different residue number in its native protein and/or (ii) a different native amino acid in its native protein.

Likewise, FIG. 2 shows that equivalent positions can be identified in fragments (e.g. domains) of an albumin with reference to SEQ ID NO: 2 (HSA).

### Conventions for Designation of Variants

For purposes of the present invention, the mature polypeptide disclosed in SEQ ID NO: 2 is used to determine the corresponding amino acid residue in another albumin. The amino acid sequence of another albumin is aligned with the mature polypeptide disclosed in SEQ ID NO: 2, and based on the alignment, the amino acid position number corresponding to any amino acid residue in the mature polypeptide disclosed in SEQ ID NO: 2 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another albumin can be determined or confirmed by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

When the other polypeptide (or protein) has diverged from the mature polypeptide of SEQ ID NO: 2 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more (several) representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing the albumin variants of the present invention, the nomenclature described below is adapted for ease of reference. The accepted IUPAC single letter or three letter amino acid abbreviation is employed. The term ‘point mutation’ and/or ‘alteration’ includes deletions, insertions and substitutions.

Substitutions.

For an amino acid substitution, the following nomenclature is used: Original amino acid, position, substituted amino acid. Accordingly, the substitution of threonine at position 226 with alanine is designated as “Thr226Ala” or “T226A”. Multiple mutations (or alterations) are separated by addition marks (“+”), e.g, “Gly205Arg+Ser411Phe” or “G205R+S411F”, representing substitutions at positions 205 and 411 of glycine (G) with arginine (R) and serine (S) with phenylalanine (F), respectively. The Figures also use (“/”), e.g, “E492T/N503D” this should be viewed as interchangeable with (“+”).

Deletions.

For an amino acid deletion, the following nomenclature is used: Original amino acid, position*. Accordingly, the deletion of glycine at position 195 is designated as “Gly195*” or “G195*”. Multiple deletions are separated by addition marks (“+”), e.g, “Gly195*+Ser411*” or “G195*+S411*”.

Insertions.

As disclosed above, an insertion may be to the N-side (‘upstream’, ‘X−1’) or C-side (‘downstream’, ‘X+1’) of the amino acid occupying a position (‘the named (or original) amino acid’, ‘X’).

For an amino acid insertion to the C-side (‘downstream’, ‘X+1’) of the original amino acid (‘X’), the following nomenclature is used: Original amino acid, position, original amino acid, inserted amino acid. Accordingly the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G-K-A |

For an amino acid insertion to the N-side (‘upstream’, ‘X−1’) of the original amino acid (X), the following nomenclature is used: Original amino acid, position, inserted amino acid, original amino acid. Accordingly the insertion of lysine (K) before glycine (G) at position 195 is designated “Gly195LysGly” or “G195KG”. An insertion of multiple amino acids is designated [Original amino acid, position, inserted amino acid #1, inserted amino acid #2; etc., original amino acid]. For example, the insertion of lysine (K) and alanine (A) before glycine at position 195 is indicated as “Gly195LysAlaGly” or “G195KAG”. In such cases the inserted amino acid residue(s) are numbered by the addition of lower case letters with prime to the position number of the amino acid residue following the inserted amino acid residue(s). In the above example, the sequence would thus be:

| Parent: | Variant: |
|---|---|
| 195 | 195a′ 195b′ 195 |
| G | K-A-G |

Multiple Alterations.

Variants comprising multiple alterations are separated by addition marks (“+”), e.g, “Arg170Tyr+Gly195Glu” or “R170Y+G195E” representing a substitution of arginine and glycine at positions 170 and 195 tyrosine and glutamic acid, respectively.

Different Alterations.

Where different alterations can be introduced at a position, the different alterations are separated by a comma, e.g, “Arg170Tyr,Glu” represents a substitution of arginine at position 170 with tyrosine or glutamic acid. Thus, “Tyr167Gly,Ala+Arg170Gly,Ala” designates the following variants: “Tyr167Gly+Arg170Gly”, “Tyr167Gly+Arg170Ala”, “Tyr167Ala+Arg170Gly”, and “Tyr167Ala+Arg170Ala”.

### DETAILED DESCRIPTION OF THE INVENTION

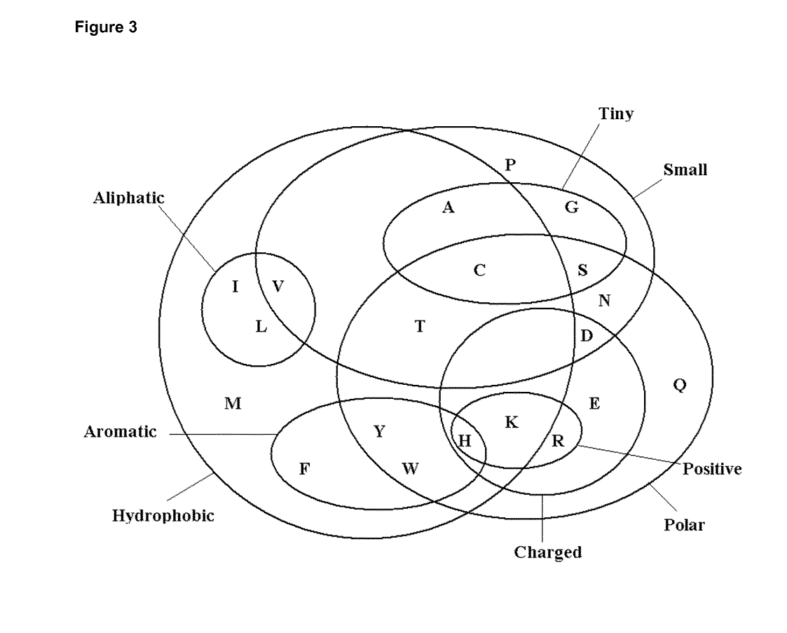

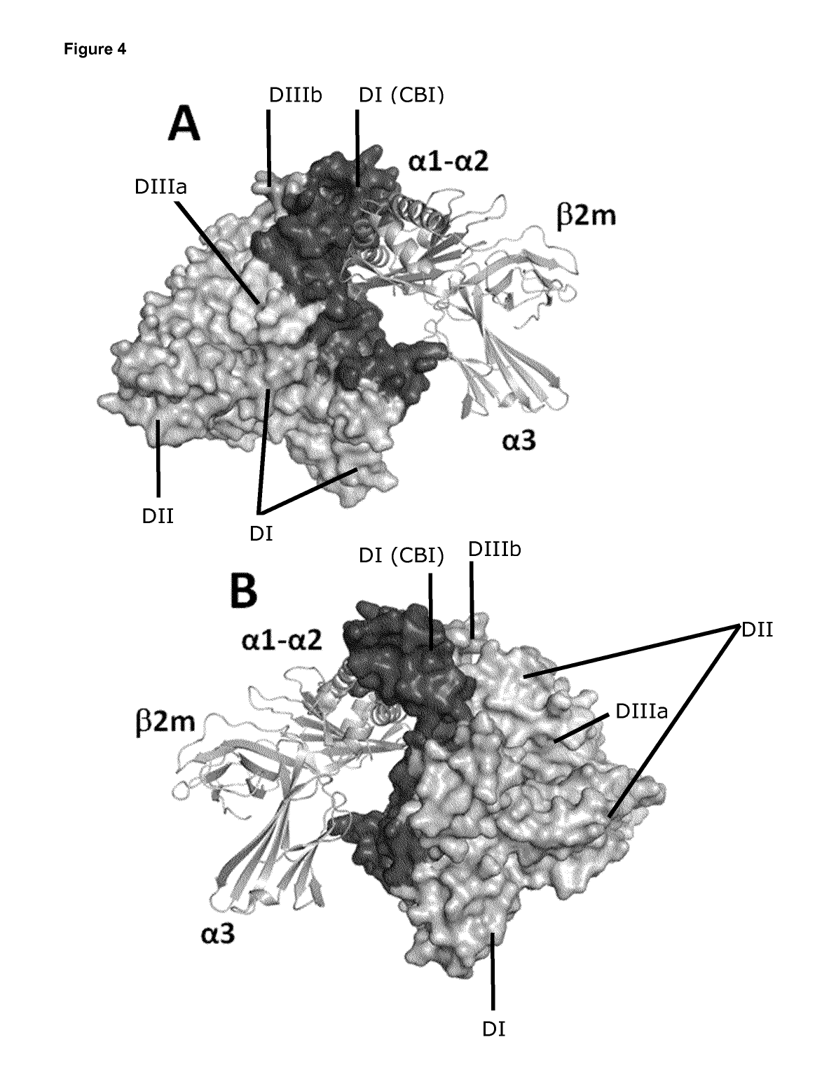

The present invention relates to albumin variants, comprising one or more (several) alterations at positions in Domain II of albumin, particularly from positions corresponding to 195 to 384 of SEQ ID NO: 2. More particularly, the invention relates to albumin variants comprising one or more (several) alterations at positions corresponding to positions comprising or selected from the group consisting of 198, 206, 340, 341, 342, 343, 344, 345, 348, 349, 352, 381, 382, 383, and/or 384 of the mature polypeptide of SEQ ID NO: 2, or at equivalent positions in other albumins or fragments thereof. The variants may have altered binding affinity FcRn compared to the binding affinity of WT albumin to FcRn. A representation of an shFcRn-HSA docking model is shown in FIG. 4.

### Variants

A first aspect of the invention provides polypeptides which are variant albumins or fragments thereof, or fusion polypeptides comprising the variant albumin or fragments thereof, of a parent albumin, comprising one or more (several) alterations at positions in Domain II of albumin particularly from positions corresponding to 195 to 384 of SEQ ID NO: 2. More particularly, the invention relates to albumin variants comprising one or more (several) alterations at positions corresponding to positions comprising or selected from the group consisting of 198, 206, 340, 341, 342, 343, 344, 345, 348, 349, 352, 381, 382, 383, and/or 384 of the mature polypeptide of SEQ ID NO: 2.

It is preferred that the parent albumin and/or the variant albumin comprises or consists of:

(a) a polypeptide having at least 60% sequence identity to the mature polypeptide of SEQ ID NO: 2;

(b) a polypeptide encoded by a polynucleotide that hybridizes under low stringency conditions with (i) the mature polypeptide coding sequence of SEQ ID NO: 1, or (ii) the full-length complement of (i);

(c) a polypeptide encoded by a polynucleotide having at least 60% identity to the mature polypeptide coding sequence of SEQ ID NO: 1; and/or

(d) a fragment of the mature polypeptide of SEQ ID NO: 2.

The variants of albumin or fragments thereof or fusion polypeptides comprising albumin or fragments thereof comprise alterations, such as substitutions, deletions or insertions at positions comprising or selected from the group consisting of 198, 206, 340, 341, 342, 343, 344, 345, 348, 349, 352, 381, 382, 383, and/or 384 of the mature polypeptide of SEQ ID NO: 2 or in equivalent positions of other albumins or variants or fragments thereof. Positions 342 to 384 form helices IIB-h3 and IIb-h4 in the structure of albumin. A stop codon may be introduced in addition to the alterations described herein and if introduced at position 574 or further downstream (e.g. in SEQ ID NO: 2) it is introduced at from position 574 to 585).

The variant albumin, a fragment thereof, or albumin part of a fusion polypeptide comprising variant albumin or a fragment thereof according to the invention has generally a sequence identity to the sequence of HSA shown in SEQ ID NO: 2 of at least 60%, preferably at least 70%, preferably at least 80%, preferably at least 85%, preferably at least 90%, more preferred at least 95%, more preferred at least 96%, more preferred at least 97%, more preferred at least 98% and most preferred at least 99%. The variant has less than 100% identity to SEQ ID NO: 2.

The variant albumin, a fragment thereof, or albumin part of a fusion polypeptide comprising variant albumin or a fragment thereof according to the invention has generally a sequence identity to the sequence of the parent albumin of at least 60%, preferably at least 70%, preferably at least 80%, preferably at least 85%, preferably at least 90%, more preferred at least 95%, more preferred at least 96%, more preferred at least 97%, more preferred at least 98% and most preferred at least 99%. The variant has less than 100% identity to the sequence of the parent albumin.

In one aspect, the number of alterations in the variants of the invention is 1 to 20, e.g, 1 to 10 and 1 to 5, such as 1, 2, 3, 4, 5, 6, 7, 8, 9, 10, 11, 12, 13, 14, 15, 16, 17, 18, 19 or 20 alterations relative to SEQ ID NO: 2 or relative to the sequence of the parent albumin.

At position 198 of SEQ ID NO: 2 (or equivalent position of other albumins or variants or fragments thereof), it is preferred that the alteration is a substitution, such as from the native amino acid to A, C, D, E, F, G, H, I, K, L, M, N, P, Q, R, S, T, V, W, Y. In SEQ ID NO: 2 the native amino acid at position 198 is L, therefore a substitution to L is not preferred. To generate an albumin variant with decreased binding affinity to FcRn (compared to the binding affinity of wild-type albumin (such as HSA) to FcRn), a substitution to A is preferred.

At position 206 of SEQ ID NO: 2 (or equivalent position of other albumins or variants or fragments thereof), it is preferred that the alteration is a substitution, such as from the native amino acid to A, C, D, E, F, G, H, I, K, L, M, N, P, Q, R, S, T, V, W, Y. In SEQ ID NO: 2 the native amino acid at position 206 is F, therefore a substitution to F is not preferred. To generate an albumin variant with decreased binding affinity to FcRn (compared to the binding affinity of wild-type albumin (such as HSA) to FcRn), a substitution to A is preferred.

At position 342 of SEQ ID NO: 2 (or equivalent position of other albumins or variants or fragments thereof), it is preferred that the alteration is a substitution, such as from the native amino acid to A, C, D, E, F, G, H, I, K, L, M, N, P, Q, R, S, T, V, W, Y. In SEQ ID NO: 2 the native amino acid at position 342 is S, therefore a substitution to S is not preferred. To generate an albumin variant with increased binding affinity to FcRn (compared to the binding affinity of wild-type albumin (such as HSA) to FcRn), a substitution to Y, W, F, H, T, N, Q, A, C, I, L, P, or V is preferred. A substitution to Y is particularly preferred.

At position 345 of SEQ ID NO: 2 (or equivalent position of other albumins or variants or fragments thereof), it is preferred that the alteration is a substitution, such as from the native amino acid to A, C, D, E, F, G, H, I, K, L, M, N, P, Q, R, S, T, V, W, Y. In SEQ ID NO: 2 the native amino acid at position 345 is L, therefore a substitution to L is not preferred. To generate an albumin variant with increased binding affinity to FcRn (compared to the binding affinity of wild-type albumin (such as HSA) to FcRn), a substitution to E, H, I or Q is preferred.

At position 349 of SEQ ID NO: 2 (or equivalent position of other albumins or variants or fragments thereof), it is preferred that the alteration is a substitution, such as from the native amino acid to A, C, D, E, F, G, H, I, K, L, M, N, P, Q, R, S, T, V, W, Y. In SEQ ID NO: 2 the native amino acid at position 349 is L, therefore a substitution to L is not preferred. To generate an albumin variant with increased binding affinity to FcRn (compared to the binding affinity of wild-type albumin (such as HSA) to FcRn), a substitution to F, W, Y, H, P, K or Q is preferred. Substitution to F is particularly preferred.

At position 381 of SEQ ID NO: 2 (or equivalent position of other albumins or variants or fragments thereof), it is preferred that the alteration is a substitution, such as from the native amino acid to A, C, D, E, F, G, H, I, K, L, M, N, P, Q, R, S, T, V, W, Y. In SEQ ID NO: 2 the native amino acid at position 381 is V, therefore a substitution to V is not preferred. To generate an albumin variant with increased binding affinity to FcRn (compared to the binding affinity of wild-type albumin (such as HSA) to FcRn), a substitution to G or A is preferred. Substitution to G is particularly preferred.

At position 384 of SEQ ID NO: 2 (or equivalent position of other albumins or variants or fragments thereof), it is preferred that the alteration is a substitution, such as from the native amino acid to A, C, D, E, F, G, H, I, K, L, M, N, P, Q, R, S, T, V, W, Y. In SEQ ID NO: 2 the native amino acid at position 384 is P, therefore a substitution to P is not preferred.

An albumin variant may comprise an alteration at one or more (several) of positions corresponding to position 198, 206, 340, 341, 342, 343, 344, 345, 348, 349, 352, 381, 382, 383, and/or 384, of SEQ ID NO: 2 and further comprise an alteration at a position corresponding to position 83 and/or 573 of SEQ ID NO: 2.

At position 83 of SEQ ID NO: 2 (or equivalent position of other albumins or variants or fragments thereof), it is preferred that the alteration is a substitution, such as from the native amino acid to A, C, D, E, F, G, H, I, K, L, M, N, P, Q, R, S, T, V, W, Y, more preferred to N, K or S, most preferred to N. In SEQ ID NO: 2 the native amino acid at position 83 is T, therefore a substitution to T is not preferred.

At position 573 of SEQ ID NO: 2 (or equivalent position of other albumins or variants or fragments thereof), it is preferred that the alteration is a substitution, such as from the native amino acid to A, C, D, E, F, G, H, I, K, L, M, N, P, Q, R, S, T, V, W, Y, more preferred to P, Y, W, H, F, T, I or V, even more preferred to P, Y or W and most preferred to P. In SEQ ID NO: 2 the native amino acid at position 573 is K, therefore a substitution to K is not preferred.

A variant albumin may comprise alterations at positions 198+573 of SEQ ID NO: 2 (or equivalent position of other albumins or variants or fragments thereof).

A variant albumin may comprise alterations at positions 206+573 of SEQ ID NO: 2 (or equivalent position of other albumins or variants or fragments thereof).

A variant albumin may comprise alterations at positions 342+573 of SEQ ID NO: 2 (or equivalent position of other albumins or variants or fragments thereof).

A variant albumin may comprise alterations at positions 345+573 of SEQ ID NO: 2 (or equivalent position of other albumins or variants or fragments thereof).

A variant albumin may comprise alterations at positions 349+573 of SEQ ID NO: 2 (or equivalent position of other albumins or variants or fragments thereof).

A variant albumin may comprise alterations at positions 381+573 of SEQ ID NO: 2 (or equivalent position of other albumins or variants or fragments thereof).

A variant albumin may comprise alterations at positions 384+573 of SEQ ID NO: 2 (or equivalent position of other albumins or variants or fragments thereof).

A variant albumin may comprise alterations at positions 83+342 of SEQ ID NO: 2 (or equivalent position of other albumins or variants or fragments thereof).

A variant albumin may comprise alterations at positions 83+345 of SEQ ID NO: 2 (or equivalent position of other albumins or variants or fragments thereof).

A variant albumin may comprise alterations at positions 83+349 of SEQ ID NO: 2 (or equivalent position of other albumins or variants or fragments thereof).

A variant albumin may comprise alterations at positions 83+381 of SEQ ID NO: 2 (or equivalent position of other albumins or variants or fragments thereof).

A variant albumin may comprise alterations at positions 83+384 of SEQ ID NO: 2 (or equivalent position of other albumins or variants or fragments thereof).

A variant albumin may comprise alterations at positions 83+342+573 of SEQ ID NO: 2 (or equivalent position of other albumins or variants or fragments thereof), e.g. T83N, K or S+S342Y, W, F, H, T, N, or Q, A, C, I, L, P, V or Y+K573P, Y or W, especially T83N+S342Y+K573P (SEQ ID NO: 103).

A variant albumin may comprise alterations at positions 83+345+573 of SEQ ID NO: 2 (or equivalent position of other albumins or variants or fragments thereof).

A variant albumin may comprise alterations at positions 83+349+573 of SEQ ID NO: 2 (or equivalent position of other albumins or variants or fragments thereof) e.g. T83N, K or S+L349F, W, Y, H, P, K or Q+K573P, Y or W, especially T83N+L349F+K573P (SEQ ID NO: X104.

A variant albumin may comprise alterations at positions 83+381+573 of SEQ ID NO: 2 (or equivalent position of other albumins or variants or fragments thereof), e.g. T83N, K or S+V381, G or A+K573P, Y or W, especially T83N+V381G+K573P (SEQ ID NO: 105).

A variant albumin may comprise alterations at positions 83+384+573 of SEQ ID NO: 2 (or equivalent position of other albumins or variants or fragments thereof).

It is preferred that the variant albumin, a fragment thereof or fusion polypeptide comprising the variant albumin or fragment thereof has altered binding affinity to FcRn and/or an altered plasma half-life compared with the corresponding parent or reference albumin, fragment thereof, or fusion polypeptide comprising the variant albumin or fragment thereof and/or an altered binding affinity to FcRn.

In a particularly preferred embodiment the parent or reference albumin is HSA (SEQ ID NO: 2) and the variant albumin, a fragment thereof or fusion polypeptide comprising the variant albumin or fragment thereof has altered binding affinity to FcRn and/or an altered plasma half-life compared with the HSA, the corresponding fragment or fusion polypeptide comprising HSA or fragment thereof and/or an altered binding affinity to FcRn.

The correlation between binding of albumin to its receptor and plasma half-life has been realized by the present inventors based on the natural occurring allele of HSA D494N. The inventors have previously analyzed this allele and found that it has a lower affinity to its receptor FcRn than the affinity of WT HSA to FcRn.

Further, it has been disclosed that a transgenic mouse having the natural mouse FcRn replaced with human FcRn has a higher serum albumin level than normal mouse (J Exp Med. (2003) 197(3):315-22). It has previously been discovered that human FcRn has a higher affinity to mouse serum albumin than mouse FcRn has to mouse serum albumin and, therefore, the observed increase in serum albumin in the transgenic mice corresponds with a higher affinity between serum albumin and its receptor, confirming the correlation between albumin binding to FcRn and plasma half-life. In addition, variants of albumin that have little or no binding to FcRn have been shown to have reduced half-life in a mouse model, Kenanova et al. (2009) J. Nucl. Med.; 50 (Supplement 2):1582).

One way to determine whether the affinity of a variant albumin to FcRn is higher or lower than the parent or reference albumin is to use the Surface Plasmon Resonance assay (SPR) as described below. The skilled person will understand that other methods might be useful to determine whether the affinity of a variant albumin to FcRn is higher or lower than the affinity of the parent or reference albumin to FcRn, e.g, determination and comparison of the binding constants KD. The binding affinity (KD) between a first molecule (e.g. ligand) and a second molecule (e.g. receptor) is a function of the kinetic constants for association (on rate, kₐ) and dissociation (off-rate, k_{d}) according to KD=k_{d}/kₐ. Thus, according to the invention variant albumins having a KD that is lower than the KD for natural HSA is considered to have a higher plasma half-life than HSA and variant albumins having a KD that is higher than the KD for natural HSA is considered to have a lower plasma half-life than HSA.

In an embodiment of the invention, the variants of albumin or fragments thereof, or fusion polypeptides comprising variant albumin or a fragment thereof according to the invention have a plasma half-life that is longer than the plasma half-life of the parent or reference albumin fragment thereof or fusion polypeptide comprising the parent or reference albumin or a fragment thereof and/or an stronger binding affinity to FcRn.

In a further embodiment the variants of albumin or fragments thereof, or fusion polypeptides comprising variant albumin or fragments thereof according to the invention have a plasma half-life that is shorter than the plasma half-life of the parent or reference albumin fragment thereof or fusion polypeptide comprising the parent or reference albumin or a fragment thereof and/or an weaker binding affinity to FcRn.

In addition to alterations at one or more (several) positions 198, 206, 340, 341, 342, 343, 344, 345, 348, 349, 352, 381, 382, 383, and/or 384 (or equivalent position of other albumins or variants of fragments thereof) the variant albumin or fragments thereof, or fusion polypeptides comprising variant albumin or fragments thereof according to the invention may contain additional substitutions, deletions or insertions in other positions of the molecules. Such additional substitutions, deletions or insertions may be useful in order to alter other properties of the molecules such as but not limited to altered glycosylation; introduction of reactive groups of the surface such a thiol groups, removing/generating a carbamoylation site; etc.

Residues that might be altered in order to provide reactive residues on the surface and which advantageously could be applied to the invention has been disclosed in WO2010/092135 (incorporated herein by reference). Particular preferred residues include the positions corresponding to positions in SEQ ID NO: 2.

As examples of alterations that can be made in SEQ ID NO: 2 or in corresponding positions in other albumins in order to provide a reactive thiol group on the surface includes alterations corresponding to following alterations in SEQ ID NO: 2: L585C, D1C, A2C, D562C, A364C, A504C, E505C, T79C, E86C, D129C, D549C, A581C, D121C, E82C, S270C, A578C, L595LC, D1 DC, A2AC, D562DC, A364AC, A504AC, E505EC, T79TC, E86EC, D129DC, D549DC, A581AC, A581AC, D121DC, E82EC, S270SC, A579AC, C360*, C316*, C75*, C168*, C558*, C361*, C91*, C124*, C169* and C567*. Alternatively a cysteine residue may be added to the N or C terminal of albumin. The term ‘reactive thiol’ means and/or includes a thiol group provided by a Cys which is not disulphide bonded to a Cysteine and/or which is sterically available for binding to a partner such as a conjugation partner.

### Fusion Polypeptides

A second aspect of the invention relates to fusion polypeptides. Therefore, the variants of albumin or fragments thereof according to the invention may be fused with a non-albumin polypeptide fusion partner. The fusion partner may in principle be any polypeptide but generally it is preferred that the fusion partner is a polypeptide having therapeutic, prophylactic (including vaccine), diagnostic, imaging or other beneficial properties. Such properties may be referred to as ‘pharmaceutically beneficial properties’. Fusion polypeptides comprising albumin or fragments thereof are known in the art. It has been found that such fusion polypeptides comprising albumin or a fragment thereof and a fusion partner polypeptide have a longer plasma half-life compared to the unfused fusion partner polypeptide alone. According to the invention it is possible to alter the plasma half-life of the fusion polypeptides according to the invention compared to the corresponding fusion polypeptides of the prior art. ‘Alter’ includes both increasing the plasma half-life or decreasing the plasma half-life. Increasing the plasma half-life is preferred. The invention allows tailoring of half-life to a term desired.

One or more (several) therapeutic, prophylactic (including vaccine), diagnostic, imaging or other beneficial polypeptides may be fused to the N-terminus, the C-terminus of albumin, inserted into a loop in the albumin structure or any combination thereof. It may or it may not comprise linker sequences separating the various components of the fusion polypeptide.

Teachings relating to fusions of albumin or a fragment thereof are known in the art and the skilled person will appreciate that such teachings can also be applied to the invention. WO 2001/79271A (particularly page 9 and/or Table 1), WO 2003/59934 (particularly Table 1), WO03/060071 (particularly Table 1) and WO01/079480 (particularly Table 1) (each incorporated herein by reference in their entirety) also contain examples of therapeutic, prophylactic (including vaccine), diagnostic, imaging or other beneficial polypeptides that may be fused to albumin or fragments thereof, and these examples apply also to the invention.

Further preferences for the second aspect of the invention include those of the first aspect of the invention and those provided below the twelfth aspect of the invention. The skilled person understands that any aspect of the invention may be combined with another aspect or aspects of the invention and/or with one or more (several) of the preferences for the aspects of the invention and/or other disclosures made herein.

### Polynucleotides

A third aspect of the invention relates to isolated polynucleotides that encode any of the variants or fusion polypeptides of the invention. The polynucleotide may be an isolated polynucleotide. The polynucleotide may be comprised in a vector (such as a plasmid) and/or in a host cell.

Further preferences for the third aspect of the invention include those of the first aspect of the invention and those provided below the twelfth aspect of the invention. The skilled person understands that any aspect of the invention may be combined with another aspect or aspects of the invention and/or with one or more (several) of the preferences for the aspects of the invention and/or other disclosures made herein.

### Nucleic Acid Constructs

A fourth aspect of the invention relates to nucleic acid constructs comprising a polynucleotide encoding a variant or fusion polypeptide of the invention operably linked to one or more (several) control sequences that direct the expression of the coding sequence in a suitable host cell under conditions compatible with the control sequences.

A polynucleotide may be manipulated in a variety of ways to provide for expression of a variant. Manipulation of the polynucleotide prior to its insertion into a vector may be desirable or necessary depending on the expression vector. The techniques for modifying polynucleotides utilizing recombinant DNA methods are well known in the art.

The control sequence may be a promoter sequence, which is recognized by a host cell for expression of the polynucleotide. The promoter sequence contains transcriptional control sequences that mediate the expression of the variant. The promoter may be any nucleic acid sequence that shows transcriptional activity in the host cell including mutant, truncated, and hybrid promoters, and may be obtained from genes encoding extracellular or intracellular polypeptides either homologous or heterologous to the host cell.

In a yeast host, useful promoters are obtained from the genes for Saccharomyces cerevisiae enolase (ENO-1), Saccharomyces cerevisiae protease A (PRA1), Saccharomyces cerevisiae protease B (PRB1), Saccharomyces cerevisiae translation elongation factor (TEF1), Saccharomyces cerevisiae translation elongation factor (TEF2), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The skilled person knows useful promoters for use in rice and mammalian cells, such as CHO or HEK. In a rice host, useful promoters are obtained from cauliflower mosaic virus 35S RNA gene (CaMV35S), maize alcohol dehydrogenase (Adh1) and alpha Amy3.

In a mammalian host cell, such as CHO or HEK, useful promoters are obtained from Cytomegalovirus (CMV) and CAG hybrid promoter (hybrid of CMV early enhancer element and chicken beta-actin promoter), Simian vacuolating virus 40 (SV40).

The control sequence may also be a suitable transcription terminator sequence, which is recognized by a host cell to terminate transcription. The terminator sequence is operably linked to the 3′-terminus of the polynucleotide encoding the variant. Any terminator that is functional in the host cell may be used.

Preferred terminators for yeast host cells are obtained from the genes for Saccharomyces cerevisiae enolase, Saccharomyces cerevisiae cytochrome C (CYC1), Saccharomyces cerevisiae alcohol dehydrogenase (ADH1) and Saccharomyces cerevisiae glyceraldehyde-3-phosphate dehydrogenase. Other useful terminators for yeast host cells are described by Romanos et al., 1992, supra. The skilled person knows useful terminators for use in rice and mammalian cells, such as CHO or HEK. For example, in a rice host, preferred terminators are obtained from Agrobacterium tumefaciens nopaline synthase (Nos) and cauliflower mosaic virus 35S RNA gene (CaMV35S).

The control sequence may also be a suitable leader sequence, a nontranslated region of an mRNA that is important for translation by the host cell. The leader sequence is operably linked to the 5′-terminus of the polynucleotide encoding the variant. Any leader sequence that is functional in the host cell may be used.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the variant-encoding sequence and, when transcribed, is recognized by the host cell as a signal to add polyadenosine residues to transcribed mRNA. Any polyadenylation sequence that is functional in the host cell may be used.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding region naturally linked in translation reading frame with the segment of the coding region that encodes the variant. Alternatively, the 5′-end of the coding sequence may contain a signal peptide coding region that is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the variant. However, any signal peptide coding region that directs the expressed variant into the secretory pathway of a host cell may be used.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra. The skilled person knows useful signal peptides for use in rice and mammalian cells, such as CHO or HEK.

Where both signal peptide and propeptide regions are present at the N-terminus of a variant, the propeptide region is positioned next to the N-terminus of the variant and the signal peptide region is positioned next to the N-terminus of the propeptide region.

Further preferences for the fourth aspect of the invention include those of the first aspect of the invention and those provided below the twelfth aspect of the invention. The skilled person understands that any aspect of the invention may be combined with another aspect or aspects of the invention and/or with one or more (several) of the preferences for the aspects of the invention and/or other disclosures made herein.

### Preparation of Variants

A fifth aspect of the invention relates to a method for preparing or obtaining a variant albumin or fragment thereof, or fusion polypeptides comprising the variant albumin or fragments thereof, or associates of variant albumin or fragment thereof comprising:

(a) introducing into a parent albumin or fragments thereof, or fusion polypeptides comprising the parent albumin or fragments thereof an alteration in Domain II such as at one or more (several) positions corresponding to positions 198, 206, 340, 342, 343, 345, 348, 349, 352, 381, 383, and/or 384 of SEQ ID NO: 2; and (b) recovering the variant albumin or fragment thereof, or fusion polypeptides comprising the variant albumin or fragment thereof.

Preferred alterations are as described in relation to the first aspect of the invention. The resultant variant albumin or fragment thereof may have altered FcRn-binding affinity compared to the FcRn-binding affinity of a reference such as a parent albumin or fragment which does not comprise the alterations. More preferably, the resultant variant albumin or fragment thereof has a stronger FcRn-binding affinity.

The invention includes a method for preparing a polypeptide which is a variant of albumin, fragment thereof or fusion polypeptide comprising said variant albumin or fragment thereof having a binding affinity to FcRn which is altered compared to the binding affinity of a reference albumin, fragment or fusion thereof to FcRn, comprising:

(a) providing a nucleic acid encoding a parent albumin such as an albumin having at least 60% sequence identity to SEQ ID NO: 2;

(b) modifying the sequence of step (a), to encode a polypeptide which is a variant albumin, fragment thereof or fusion polypeptide comprising said variant albumin or fragment thereof comprising alterations at positions corresponding to positions 198, 206, 340, 341, 342, 343, 344, 345, 348, 349, 352, 381, 382, 383, and/or 384 in SEQ ID NO: 2;

(c) optionally, introducing the modified sequence of step (b) in a suitable host cell;

(d) optionally, growing the cells in a suitable growth medium under condition leading to expression of the polypeptide; and

(e) optionally, recovering the polypeptide from the growth medium;

wherein the polypeptide has an altered binding affinity to FcRn and/or an altered plasma half-life compared with the half-life of a parent albumin, reference albumin, fragment thereof or fusion polypeptide comprising said parent albumin, reference albumin or fragment or fusion thereof.

It is preferred that the parent albumin and/or the variant albumin comprises or consists of:

(a) a polypeptide having at least 60% sequence identity to the mature polypeptide of SEQ ID NO: 2;

(b) a polypeptide encoded by a polynucleotide that hybridizes under low stringency conditions with (i) the mature polypeptide coding sequence of SEQ ID NO: 1, or (ii) the full-length complement of (i);

(c) a polypeptide encoded by a polynucleotide having at least 60% identity to the mature polypeptide coding sequence of SEQ ID NO: 1; and/or

(d) a fragment of the mature polypeptide of SEQ ID NO: 2.

The variants can be prepared by those skilled persons using any mutagenesis procedure known in the art, such as site-directed mutagenesis, synthetic gene construction, semi-synthetic gene construction, random mutagenesis, shuffling, etc.

Site-directed mutagenesis is a technique in which one or more (several) mutations (alterations) are created at one or more (several) defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests at the plasmid and the oligonucleotide is the same, permitting ligation of the plasmid and insert to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art, see, e.g, U.S. Patent Application Publication: 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Any site-directed mutagenesis procedure can be used in the invention. There are many commercial kits available that can be used to prepare variants.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g, Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide sub sequences may then be shuffled.

Further preferences for the fifth aspect of the invention include those of the first aspect of the invention and those provided below the twelfth aspect of the invention. The skilled person understands that any aspect of the invention may be combined with another aspect or aspects of the invention and/or with one or more (several) of the preferences for the aspects of the invention and/or other disclosures made herein.

### Methods of Production

A sixth aspect of the invention relates to methods of preparation of a variant according to the invention. The variants of the invention can be prepared using techniques well known to the skilled person. One convenient way is by cloning nucleic acid encoding the parent albumin or a fragment thereof or fusion polypeptide comprising albumin or a fragment thereof, modifying said nucleic acid to introduce the desired substitution(s) at one or more (several) positions corresponding to positions 198, 206, 340, 341, 342, 343, 344, 345, 348, 349, 352, 381, 382, 383, and/or 384 of the mature polypeptide of SEQ ID NO: 2 (or equivalent positions in other albumins or fragments thereof), preparing a suitable genetic construct where the modified nucleic acid is placed in operative connection with suitable regulatory genetic elements, such as promoter, terminator, activation sites, ribosome binding sites etc., introducing the genetic construct into a suitable host organism, culturing the transformed host organism under conditions leading to expression of the variant and recovering the variant. All these techniques are known in the art and it is within the skills of the average practitioner to design a suitable method for preparing a particular variant according to the invention.

The variant polypeptide of the invention may also be connected to a signal sequence in order to have the variant polypeptide secreted into the growth medium during culturing of the transformed host organism. It is generally advantageous to have the variant polypeptide secreted into the growth medium in order to ease recovery and purification.

Techniques for preparing variant polypeptides have also been disclosed in WO 2009019314 (included by reference) and these techniques may also be applied to the invention.

Albumins have been successfully expressed as recombinant proteins in a range of hosts including fungi (including but not limited to Aspergillus (WO06066595), Kluyveromyces (Fleer 1991, Bio/technology 9, 968-975), Pichia (Kobayashi 1998 Therapeutic Apheresis 2, 257-262) and Saccharomyces (Sleep 1990, Bio/technology 8, 42-46)), bacteria (Pandjaitab 2000, J. Allergy Clin. Immunol. 105, 279-285)), animals (Barash 1993, Transgenic Research 2, 266-276) and plants (including but not limited to potato and tobacco (Sijmons 1990, Bio/technology 8, 217 and Farran 2002, Transgenic Research 11, 337-346) and rice e.g. Oryza sativa) and mammalian cells such as CHO and HEK. The variant polypeptide of the invention is preferably produced recombinantly in a suitable host cell. In principle any host cell capable of producing a polypeptide in suitable amounts may be used and it is within the skills of the average practitioner to select a suitable host cell according to the invention. A preferred host organism is yeast, preferably selected among Saccharomycacae, more preferred Saccharomyces cerevisiae.

The variant polypeptides of the invention may be recovered and purified from the growth medium using a combination of known separation techniques such as filtration, centrifugation, chromatography, and affinity separation techniques etc. It is within the skills of the average practitioner to purify the variants of the invention using a particular combination of such known separation steps. As an example of purification techniques that may be applied to the variants of the invention can be mentioned the teaching of WO00/44772.

The variant polypeptides of the invention may be used for delivering a therapeutically beneficial compound (including prophylactically beneficial compound such as a vaccine) to an animal or a human individual in need thereof. Such therapeutically beneficial compounds include, but are not limited, to labels and readily detectable compounds for use in diagnostics, such as various imaging techniques; pharmaceutical active compounds such as drugs, or specifically binding moieties such as antibodies. The variants of the invention may even be connected to two or more (several) different therapeutically beneficial compounds, e.g, an antibody and a drug, which gives the combined molecule the ability to bind specifically to a desired target and thereby provide a high concentration of the connected drug at that particular target.

Further preferences for the sixth aspect of the invention include those of the first aspect of the invention and those provided below the twelfth aspect of the invention. The skilled person understands that any aspect of the invention may be combined with another aspect or aspects of the invention and/or with one or more (several) of the preferences for the aspects of the invention and/or other disclosures made herein.

### Conjugates

A seventh aspect of the invention relates to conjugates (conjugations). Therefore, the variants of albumin or fragments thereof or fusion polypeptides according to the invention may be conjugated to a second molecule (‘conjugation partner’) using techniques known within the art. The conjugation partner may be a therapeutic, prophylactic (including vaccine), diagnostic, imaging or other beneficial moiety. Said conjugation partner may be a polypeptide or a non-polypeptide chemical. The conjugation partner may be a polypeptide, chemical (e.g. chemically synthesized drug) or a nucleic acid (e.g. DNA, RNA, siRNA).

Said second molecule may comprise a diagnostic or imaging moiety, and in this embodiment the conjugate may be useful as a diagnostic tool such as in imaging; or the second molecule may be a therapeutic or prophylactic (e.g. vaccine) compound and in this embodiment the conjugate may be used for therapeutic or prophylactic (e.g. vaccination) purposes where the conjugate will have the therapeutic or prophylactic properties of the therapeutic or prophylactic compound as well as the desirable plasma half-life provided by the albumin part of the conjugate. Conjugates of albumin and a therapeutic molecule are known in the art and it has been verified that such conjugates have long plasma half-life compared with the non-conjugated, free therapeutic molecule as such. According to the invention it is possible to alter the binding affinity to FcRn and/or plasma half-life of the conjugate according to the invention compared to the corresponding conjugates of the prior art. ‘Alter’ includes both increasing the plasma half-life and decreasing the plasma half-life binding affinity to FcRn and/or increasing the binding affinity and decreasing the binding affinity to FcRn. Increasing the plasma half-life and/or binding affinity to FcRn is preferred. The conjugates may conveniently be linked via a free thiol group present on the surface of HSA (amino acid residue 34 of mature HSA) using well known chemistry.

In one particular preferred aspect the variant albumin or fragment thereof is conjugated to a beneficial therapeutic or prophylactic (including vaccine) compound and the conjugate is used for treatment of a condition in a patient in need thereof, which condition is responsive to the particular selected therapeutic compound. Techniques for conjugating such a therapeutically useful compound to the variant albumin or fragment thereof are known in the art. WO 2009/019314 (incorporated herein by reference in its entirety) discloses examples of techniques suitable for conjugating a therapeutically compound to a polypeptide which techniques can also be applied to the invention. Further WO 2009/019314 discloses examples of compounds and moieties that may be conjugated to substituted transferrin and these examples may also be applied to the invention. The teaching of WO 2009/019314 is included herein by reference.

HSA contains in its natural form one free thiol group (at Cys34) that conveniently may be used for conjugation. As a particular embodiment within this aspect the variant albumin or fragment thereof may comprise further modifications provided to generate additional free thiol groups on the surface. This has the benefit that the payload of the variant albumin or fragment thereof is increased so that more than one molecule of the therapeutic (e.g. prophylactic) compound can be conjugated to each molecule of variant albumin or fragment thereof, or two or more (several) different therapeutic compounds may be conjugated to each molecule of variant albumin or fragment thereof, e.g, a compound having targeting properties such as an antibody specific for example a tumor; and a cytotoxic drug conjugated to the variant albumin or fragment thereof thereby creating a highly specific drug against a tumor. Teaching of particular residues that may be modified to provide for further free thiol groups on the surface can be found in co-pending patent application WO 2010/092135, which is incorporated by reference.

The conjugation partner may alternatively be conjugated to a fusion polypeptide (described herein), resulting in a molecule comprising a fusion partner fused to the albumin as well as a conjugation partner conjugated to the same albumin or even to the fusion partner.

Further preferences for the seventh aspect of the invention include those of the first aspect of the invention and those provided below the twelfth aspect of the invention. The skilled person understands that any aspect of the invention may be combined with another aspect or aspects of the invention and/or with one or more (several) of the preferences for the aspects of the invention and/or other disclosures made herein.

### Associates

An eighth aspect of the invention relates to associates. Therefore, the variants of albumin or fragments thereof or fusion polypeptides may further be used in form of “associates”. In this connection the term “associate” is intended to mean a compound comprising a variant of albumin or a fragment thereof and another compound bound or associated to the variant albumin or fragment thereof by non-covalent binding. As an example of such an associate can be mentioned an associate consisting of variant albumin and a lipid associated to albumin by a hydrophobic interaction. Such associates are known in the art and they may be prepared using well known techniques. As an example of a preferred associate according to the invention can be mentioned, an associate comprising variant albumin and a taxane, a taxol or taxol derivative (e.g. paclitaxel). Further examples of associates comprise a therapeutic, prophylactic (including vaccine), diagnostic, imaging or other beneficial moiety.

The half-life of an albumin associate according to the invention may be longer or shorter than the half-life of the ‘other compound’ alone. The half-life of an albumin associate according to the invention may be longer or shorter than the half-life of the analogous/equivalent albumin associate comprising or consisting of a reference albumin such as native HSA (instead of an albumin variant or derivative according to the invention) and the ‘other compound’. Likewise, the binding affinity to FcRn of an albumin associate according to the invention may be stronger or weaker than the binding affinity to FcRn of the analogous/equivalent albumin associate comprising or consisting of a reference albumin such as native HSA (instead of an albumin variant or derivative according to the invention) and the ‘other compound’. Methods for the preparation of associates are well-known to the skilled person, for example, formulation (by association) of HSA with Lipo-compounds is described in Hussain, R. and Siligardi, G. (2006) International Journal of Peptide Research and Therapeutics, Vol. 12, NO: 3, pp. 311-315.

Further preferences for the eighth aspect of the invention include those of the first aspect of the invention and those provided below the twelfth aspect of the invention. The skilled person understands that any aspect of the invention may be combined with another aspect or aspects of the invention and/or with one or more (several) of the preferences for the aspects of the invention and/or other disclosures made herein.

### Compositions

A ninth aspect of the invention relates to compositions, for example pharmaceutical compositions. Therefore the invention is also directed to the use of a variant of albumin or a fragment thereof or fusion polypeptides comprising variant albumin or fragments thereof, or a conjugate comprising a variant of albumin or a fragment thereof, or an associate comprising a variant of albumin or a fragment thereof for the manufacture of a pharmaceutical composition, wherein the variant of albumin or a fragment thereof or fusion polypeptides comprising variant albumin or fragments thereof, or a conjugate comprising a variant of albumin or a fragment thereof, or an associate comprising a variant of albumin or a fragment thereof has an altered binding affinity to FcRn and/or an altered plasma half-life compared with HSA or the corresponding fragment thereof or fusion polypeptide comprising HSA or fragment thereof or conjugate comprising HSA. Likewise, the invention is also directed to use as a medicament.

In this connection the corresponding fragment of HSA is intended to mean a fragment of HSA that aligns with and has same number of amino acids as the fragment of the variant albumin with which it is compared. Similarly the corresponding fusion polypeptide comprising HSA or conjugate comprising HSA is intended to mean molecules having the same size and amino acid sequence as the fusion polypeptide of conjugate comprising variant albumin, with which it is compared.

The composition may comprise a pharmaceutically acceptable carrier or excipient such as water, Polysorbate 80 or those specified in the US Pharmacopoeia for human albumin.

Further preferences for the ninth aspect of the invention include those of the first aspect of the invention and those provided below the twelfth aspect of the invention. The skilled person understands that any aspect of the invention may be combined with another aspect or aspects of the invention and/or with one or more (several) of the preferences for the aspects of the invention and/or other disclosures made herein.

### Nanoparticles

A tenth aspect of the invention relates to a nanoparticle comprising a variant, fusion, conjugate, associate, nanoparticle, composition or polynucleotide as disclosed herein.

Techniques for incorporation of a molecule into nano- or microparticles are known in the art. Preferred methods for preparing nano- or microparticles that may be applied to the albumin, variant, fragment, fusion, conjugate or associate thereof according to the invention is disclosed in WO 2004/071536 or WO2008/007146 or Oner & Groves (Pharmaceutical Research, Vol 10(9), 1993, pages 1387 to 1388) which are incorporated herein by reference. Preferably the average diameter of a nano-particle is from 5 to 1000 nm, more preferably from 5, 10, 20, 30, 40, 50, 80, 100, 130, 150, 200, 300, 400, 500, 600, 700, 800, 900, or 999 to 5, 10, 20, 30, 40, 50, 80, 100, 130, 150, 200, 300, 400, 500, 600, 700, 800, 900, or 1000 nm. An advantage of a microparticle less than 200 nm diameter, and more particularly less than 130 nm, is that is amenable to sterilization by filtration through a 0.2 μm (micron) filter. Preferably, the average diameter of a micro-particle is from 1000 nm (1 μm (micron)) to 100 μm (micron), more preferably from 1, 2, 5, 10, 20, 30, 40, 50, 60, 70, 80, 90, 100 to 1, 2, 5, 10, 20, 30, 40, 50, 60, 70, 80, 90, 100 μm (micron).

Further preferences for the tenth aspect of the invention include those of the first aspect of the invention and those provided below the twelfth aspect of the invention. The skilled person understands that any aspect of the invention may be combined with another aspect or aspects of the invention and/or with one or more (several) of the preferences for the aspects of the invention and/or other disclosures made herein.

### Uses

An eleventh aspect of the invention relates to use of a variant albumin, fragment, fusion or conjugate thereof or nanoparticle or associate thereof. Use may be, for example, in a method of treatment, prophylaxis, diagnosis or imaging. The variant albumin or fragments thereof or fusion polypeptides comprising variant albumin or fragments thereof according to the invention have the benefit that their binding affinity to FcRn and/or plasma half-life is altered compared to the parent or reference albumin or fragments thereof or fusion polypeptides comprising parent or reference albumin or fragments thereof. This has the advantage that the binding affinity to FcRn and/or plasma half-life of conjugates comprising variant albumin or a fragment thereof or fusion polypeptide comprising variant albumin or a fragment thereof, or an associate comprising variant albumin or a fragment thereof according to the invention can be selected in accordance with the particular therapeutic purpose.

In some situations, it would be advantageous to use an albumin, variant, fragment, fusion, conjugate or associate or composition thereof having a longer plasma half-life than the reference molecule or composition since this would have the benefit that the administration of the albumin, variant, fragment, fusion, conjugate or associate or composition thereof would be needed less frequently or at a reduced dose (and consequently with fewer side effects) compared to the situation where the reference molecule or composition was used. With respect to the use of a variant, fusion, conjugate, associate, nanoparticle, composition or polynucleotide the albumin moiety may comprise one more alterations as disclosed herein.

In other situations, it would be advantageous to use an albumin, variant, fragment, fusion, conjugate or associate or composition thereof having a shorter plasma half-life than the reference molecule or composition since this would have the benefit that the administration of the albumin, variant, fragment, fusion, conjugate or associate or composition thereof can be carried out at a higher dose compared to the situation where the reference molecule or composition was used with the benefit that the administered compound clears from the recipient more quickly than if the reference molecule or composition was used. With respect to the use of a variant, fusion, conjugate, associate, nanoparticle, composition or polynucleotide the albumin moiety may comprise one more alterations as disclosed herein.

For example for a conjugate, associate or fusion polypeptide used for imaging purposes in animals or human beings, where the imaging moiety has a very short half-life and a conjugate or a fusion polypeptide comprising HSA has a plasma half-life that is far longer than needed for the imaging purposes it would be advantageous to use a variant albumin or fragment thereof of the invention having a shorter plasma half-life than the parent or reference albumin or fragment thereof, to provide conjugates of fusion polypeptides having a plasma half-life that is sufficiently long for the imaging purpose but sufficiently short to be cleared form the body of the particular patient on which it is applied.

In another example for a conjugate, an associate or fusion polypeptide comprising a therapeutic compound effective to treat or alleviate a particular condition in a patient in need for such a treatment it would be advantageous to use the variant albumin or fragment thereof having a longer plasma half-life than the parent or reference albumin or fragment thereof, to provide associates or conjugates or fusion polypeptides having longer plasma half-lives which would have the benefit that the administration of the associate or conjugate or fusion polypeptide of the invention would be needed less frequently or at reduced dose with less side effects compared to the situation where the parent or reference albumin or associates thereof or fragment thereof was used. For example, the invention provides a method of treating a proliferative disease in an individual, comprising administering the individual an effective amount of an associate according to the invention in which the associate comprises a taxane, a taxol or taxol derivative (e.g. paclitaxel).

In a further aspect the invention relates to compositions comprising the variant albumin, associates thereof or fragment thereof, variant albumin fragment or associates thereof or fusion polypeptide comprising variant albumin or fragment thereof according to the invention. The compositions are preferably pharmaceutical compositions. The composition may be prepared using techniques known in the area such as disclosed in recognized handbooks within the pharmaceutical field. Since the albumin, variant, fragment, fusion, conjugate or associate thereof has a binding affinity to FcRn and/or plasma half-life which is altered (i.e. stronger or weaker and/or longer or shorter) than that of a reference molecule, the composition also has a binding affinity to FcRn and/or altered plasma half-life relative to an equivalent composition comprising the reference molecule in place of the albumin, variant, fragment, fusion, conjugate or associate thereof as described herein. The composition may be a vaccine. The polypeptide according to the invention may be an active pharmaceutical or an excipient. Optionally, the composition is provided in unit dosage form.

Preferably the albumin, variant, fragment, fusion, conjugate or associate thereof has a plasma half-life that is longer than the plasma half-life of the reference molecule e.g. the same composition except that the albumin component (e.g. albumin, variant, fragment, fusion, conjugate or associate) is wild-type albumin (e.g. HSA) or a variant, fragment, fusion, conjugate or associate.

In a particular embodiment the compositions comprise a variant albumin or a fragment thereof according to the invention and a compound comprising a pharmaceutically beneficial moiety and an albumin binding domain (ABD). According to the invention ABD means a site, moiety or domain capable of binding to circulating albumin in vivo and thereby conferring transport in the circulation of the ABD and any compound or moiety bound to said ABD. ABD's are known in the art and have been shown to bind very tight to albumin so a compound comprising an ABD bound to albumin will to a certain extent behave as a single molecule. The inventors have realized by using the variant albumin or fragment thereof according to the invention together with a compound comprising a pharmaceutically beneficial moiety and an ABD makes it possible to alter the binding affinity to FcRn and/or plasma half-life of the compound comprising a pharmaceutically beneficial moiety and an ABD compared to the situation where said compound were injected as such in a patient having need thereof or administered in a formulation comprising natural albumin or a fragment thereof.

The variant albumin or fragments thereof, conjugates comprising variant albumin or a fragment thereof or fusion polypeptide comprising variant albumin or a fragment thereof, or an associate comprising variant albumin or a fragment thereof according to the invention may also be incorporated into nano- or microparticles using techniques well known within the art. A preferred method for preparing nano- or microparticles that may be applied to the variant albumins or fragments thereof according to the invention is disclosed in WO 2004/071536 or WO2008/007146 or Oner & Groves (Pharmaceutical Research, Vol 10(9), 1993, pages 1387 to 1388) which are incorporated herein by reference.

Further preferences for the eleventh aspect of the invention include those of the first aspect of the invention and those provided below the twelfth aspect of the invention. The skilled person understands that any aspect of the invention may be combined with another aspect or aspects of the invention and/or with one or more (several) of the preferences for the aspects of the invention and/or other disclosures made herein.

### Method for Altering the FcRn-Binding Affinity or Half-Life of a Molecule

A twelfth aspect of the invention provides a method for altering the FcRn-binding affinity or half-life of a molecule comprising:

(a) where the molecule is a polypeptide, fusing or conjugating the molecule to a polypeptide disclosed herein or to a conjugate disclosed herein; associating the molecule to a polypeptide disclosed herein or to a conjugate disclosed herein; incorporating the molecule in a nanoparticle disclosed herein or a composition disclosed herein;

(b) where the molecule is not a polypeptide, conjugating the molecule to a polypeptide disclosed herein or to a conjugate disclosed herein; associating the molecule to a polypeptide disclosed herein or to a conjugate a disclosed herein; incorporating the molecule in a nanoparticle disclosed herein or a composition disclosed herein.

Examples of ‘molecule’ include those useful in therapy, prophylaxis (including those used in vaccines either as an active pharmaceutical ingredient or as an excipient), imaging and diagnosis, such as those described herein.

Further preferences for the twelfth aspect of the invention include those of the first aspect of the invention and those provided below this twelfth aspect of the invention. The skilled person understands that any aspect of the invention may be combined with another aspect or aspects of the invention and/or with one or more (several) of the preferences for the aspects of the invention and/or other disclosures made herein.

Preferences for all aspects of the invention are provided below. The skilled person understands that any aspect of the invention may be combined with another aspect or aspects of the invention and/or with one or more (several) of the preferences for the aspects of the invention and/or other disclosures made herein.

The variant of albumin or a fragment thereof or fusion polypeptides comprising variant albumin or fragments thereof, fragment thereof, conjugate, nanoparticle, associate or composition may have a plasma half-life that is either longer or shorter, preferably longer, than the plasma half-life than a corresponding albumin or a fragment thereof or fusion polypeptides comprising albumin or fragments thereof, fragment thereof, conjugate, nanoparticle, associate or composition or a binding to FcRn that is stronger or weaker, preferably weaker. Preferably the variant of albumin or a fragment thereof or fusion polypeptides comprising variant albumin or fragments thereof, fragment thereof, conjugate, nanoparticle, associate or composition has a plasma half-life that is longer than the plasma half-life of HSA or the corresponding albumin or a fragment thereof or fusion polypeptides comprising albumin or fragments thereof, fragment thereof, conjugate, nanoparticle, associate or composition.

Alternatively, this may be expressed as the variant of albumin or a fragment thereof or fusion polypeptides comprising variant albumin or fragments thereof, fragment thereof, conjugate, nanoparticle, associate or composition having a KD to FcRn (e.g. shFcRn) that is lower than the corresponding KD for HSA to FcRn or the corresponding fragment thereof or fusion polypeptide comprising HSA or fragment thereof. Preferably, the KD for the variant of albumin or a fragment thereof or fusion polypeptides comprising variant albumin or fragments thereof, fragment thereof, conjugate, nanoparticle, associate or composition is less than 0.9×KD for HSA to FcRn, more preferred less than 0.5×KD for HSA to FcRn, more preferred less than 0.1×KD for HSA to FcRn, even more preferred less than 0.05×KD for HSA to FcRn, even more preferred less than 0.02×KD for HSA to FcRn and most preferred less than 0.01×KD for HSA to FcRn (where × means ‘multiplied by’). The KD of the variant of albumin or a fragment thereof or fusion polypeptides comprising variant albumin or fragments thereof, fragment thereof, conjugate, nanoparticle, associate or composition may be between the KD of WT albumin (e.g. SEQ ID NO: 2) for FcRn and the KD of HSA K573P (SEQ ID NO: 3) for FcRn. Such KDs represent binding affinities that are higher than the binding affinity between HSA and FcRn. A higher binding affinity indicates a longer half-life, for example plasma half-life.

Alternatively, the variant of albumin or a fragment thereof or fusion polypeptides comprising variant albumin or fragments thereof, fragment thereof, conjugate, nanoparticle, associate or composition has a plasma half-life that is shorter than the plasma half-life of HSA or the corresponding fragment thereof or fusion polypeptide comprising HSA or fragment thereof.

This may be expressed as the variant of albumin or a fragment thereof or fusion polypeptides comprising variant albumin or fragments thereof, fragment thereof, conjugate, nanoparticle, associate or composition having a KD to FcRn that is higher than the corresponding KD for HSA to FcRn or the corresponding of albumin or a fragment thereof or fusion polypeptides comprising albumin or fragments thereof, fragment thereof, conjugate, nanoparticle, associate or composition. Preferably, the KD for the variant of albumin or a fragment thereof or fusion polypeptides comprising variant albumin or fragments thereof, fragment thereof, or a conjugate comprising a variant of albumin or a fragment thereof is more than 2×KD for HSA to FcRn, more preferred more than 5×KD for HSA to FcRn, more preferred more than 10×KD for HSA to FcRn, even more preferred more than 25×KD for HSA to FcRn, even most preferred more than 50×KD for HSA to FcRn. The variant of albumin or a fragment thereof or fusion polypeptides comprising variant albumin or fragments thereof, fragment thereof, conjugate, nanoparticle, associate or composition may be a null binder to FcRn.

The variant of albumin or a fragment thereof or fusion polypeptides comprising variant albumin or fragments thereof, fragment thereof, or a conjugate or nanoparticle or associate or composition comprising a variant of albumin or a fragment thereof is preferably the variant of albumin or a fragment thereof or fusion polypeptides comprising variant albumin or fragments thereof, fragment thereof, or a conjugate or nanoparticle or associate or composition comprising a variant of albumin or a fragment thereof according to the invention. A lower binding affinity indicates a shorter half-life, for example plasma half-life.

One advantage of the invention is that it allows the half-life of albumin, a variant of albumin or a fragment thereof or fusion polypeptides comprising variant albumin or fragments thereof, fragment thereof, conjugate, nanoparticle, associate or composition to be tailored in order to achieve a binding affinity or half-life which meets the needs of the user.

When determining and/or comparing KD, one or more (and preferably all) of the following parameters may be used:

Instrument: Biacore 3000 instrument (GE Healthcare)

Flow cell: CM5 sensor chip

FcRn: human FcRn, preferably soluble human FcRn, optionally coupled to a tag such as Glutathione S Transferase (GST) or Histidine (His), most preferably His such as 6 histidine residues at the C-terminus of the beta-2-microglobulin (SEQ ID NO: 31).

Quantity of FcRn: 1200-2500 RU

Coupling chemistry: amine coupling chemistry (e.g. as described in the protocol provided by the manufacturer of the instrument).

Coupling method: The coupling may be performed by injecting 20 μg/ml of the protein in 10 mM sodium acetate pH 5.0 (GE Healthcare). Phosphate buffer (67 mM phosphate buffer, 0.15 M NaCl, 0.005% Tween 20) at pH 5.5) may be used as running buffer and dilution buffer. Regeneration of the surfaces may be done using injections of HBS-EP buffer (0.01 M HEPES, 0.15 M NaCl, 3 mM EDTA, 0.005% surfactant P20) at pH 7.4 (Biacore AB).

Quantity of injection of test molecule (e.g. HSA or variant) 20-0.032 μM

Flow rate of injection: constant, e.g. 30 μl/ml

Temperature of injection: 25° C.

Data evaluation software: BIAevaluation 4.1 software (BIAcore AB).

The preferred methods for determining KD are provided in Example 3. Of the two methods, the Biacore SPR method is preferred.

The invention discloses that positions 198, 206, 340, 341, 342, 343, 344, 345, 348, 349, 352, 381, 382, 383, and/or 384 in SEQ ID NO: 2 (and therefore equivalent positions in albumins and fragments from human serum and albumin and non-human serum albumins) may be altered in order to modulate (increase of decrease) the binding affinity and/or half-life e.g. plasma half-life of an albumin, fragment, fusion, conjugate, associate, nanoparticle or composition. An alteration may be a substitution, insertion or deletion. Substitution is preferred.

A substitution or insertion may or may not comprise introduction of a conserved amino acid, i.e. conserved in relation to the amino acid at the position of interest. Examples of conserved amino acids are shown by the groups of FIG. 3: aliphatic, aromatic, hydrophobic, charged, polar, positive, tiny and small.

At position 198 of SEQ ID NO: 2 (or equivalent position of other albumins or variants or fragments thereof), it is preferred that the alteration is a substitution, such as from the native amino acid to A, C, D, E, F, G, H, I, K, L, M, N, P, Q, R, S, T, V, W, Y. In SEQ ID NO: 2 the native amino acid at position 198 is L, therefore a substitution to L is not preferred. To generate an albumin variant with decreased binding affinity to FcRn (compared to the binding affinity of wild-type albumin (such as HSA) to FcRn), a substitution to A is preferred.

At position 206 of SEQ ID NO: 2 (or equivalent position of other albumins or variants or fragments thereof), it is preferred that the alteration is a substitution, such as from the native amino acid to A, C, D, E, F, G, H, I, K, L, M, N, P, Q, R, S, T, V, W, Y. In SEQ ID NO: 2 the native amino acid at position 206 is F, therefore a substitution to F is not preferred. To generate an albumin variant with decreased binding affinity to FcRn (compared to the binding affinity of wild-type albumin (such as HSA) to FcRn), a substitution to A is preferred.

At position 342 of SEQ ID NO: 2 (or equivalent position of other albumins or variants or fragments thereof), it is preferred that the alteration is a substitution, such as from the native amino acid to A, C, D, E, F, G, H, I, K, L, M, N, P, Q, R, S, T, V, W, Y. In SEQ ID NO: 2 the native amino acid at position 342 is S, therefore a substitution to S is not preferred. To generate an albumin variant with increased binding affinity to FcRn (compared to the binding affinity of wild-type albumin (such as HSA) to FcRn), a substitution to Y, W, F, H, T, N, Q, A, C, I, L, P, V is preferred. A substitution to Y is particularly preferred.

At position 345 of SEQ ID NO: 2 (or equivalent position of other albumins or variants or fragments thereof), it is preferred that the alteration is a substitution, such as from the native amino acid to A, C, D, E, F, G, H, I, K, L, M, N, P, Q, R, S, T, V, W, Y. In SEQ ID NO: 2 the native amino acid at position 345 is L, therefore a substitution to L is not preferred. To generate an albumin variant with increased binding affinity to FcRn (compared to the binding affinity of wild-type albumin (such as HSA) to FcRn), a substitution to E is preferred.

At position 349 of SEQ ID NO: 2 (or equivalent position of other albumins or variants or fragments thereof), it is preferred that the alteration is a substitution, such as from the native amino acid to A, C, D, E, F, G, H, I, K, L, M, N, P, Q, R, S, T, V, W, Y. In SEQ ID NO: 2 the native amino acid at position 349 is L, therefore a substitution to L is not preferred. To generate an albumin variant with increased binding affinity to FcRn (compared to the binding affinity of wild-type albumin (such as HSA) to FcRn), a substitution to F, W, Y, H, P, K or Q is preferred. Substitution to F is particularly preferred.

At position 381 of SEQ ID NO: 2 (or equivalent position of other albumins or variants or fragments thereof), it is preferred that the alteration is a substitution, such as from the native amino acid to A, C, D, E, F, G, H, I, K, L, M, N, P, Q, R, S, T, V, W, Y. In SEQ ID NO: 2 the native amino acid at position 381 is V, therefore a substitution to V is not preferred. To generate an albumin variant with increased binding affinity to FcRn (compared to the binding affinity of wild-type albumin (such as HSA) to FcRn), a substitution to G or A is preferred. Substitution to G is particularly preferred.

At position 384 of SEQ ID NO: 2 (or equivalent position of other albumins or variants or fragments thereof), it is preferred that the alteration is a substitution, such as from the native amino acid to A, C, D, E, F, G, H, I, K, L, M, N, P, Q, R, S, T, V, W, Y. In SEQ ID NO: 2 the native amino acid at position 384 is P, therefore a substitution to P is not preferred.

An albumin variant may comprise an alteration at one or more (several) of positions corresponding to position 198, 206, 340, 341, 342, 343, 344, 345, 348, 349, 352, 381, 382, 383, and/or 384, of SEQ ID NO: 2 and further comprise an alteration at a position corresponding to position 83 and/or 573 of SEQ ID NO: 2.

At position 83 of SEQ ID NO: 2 (or equivalent position of other albumins or variants or fragments thereof), it is preferred that the alteration is a substitution, such as from the native amino acid to A, C, D, E, F, G, H, I, K, L, M, N, P, Q, R, S, T, V, W, Y, more preferred to N, K or S, most preferred to N. In SEQ ID NO: 2 the native amino acid at position 83 is T, therefore a substitution to T is not preferred.

At position 573 of SEQ ID NO: 2 (or equivalent position of other albumins or variants or fragments thereof), it is preferred that the alteration is a substitution, such as from the native amino acid to A, C, D, E, F, G, H, I, K, L, M, N, P, Q, R, S, T, V, W, Y, more preferred to P, Y, W, H, F, T, I or V, even more preferred to P, Y or W and most preferred to P. In SEQ ID NO: 2 the native amino acid at position 573 is K, therefore a substitution to K is not preferred.

A variant albumin may comprise alterations at positions 198+573 of SEQ ID NO: 2 (or equivalent position of other albumins or variants or fragments thereof).

A variant albumin may comprise alterations at positions 206+573 of SEQ ID NO: 2 (or equivalent position of other albumins or variants or fragments thereof).

A variant albumin may comprise alterations at positions 342+573 of SEQ ID NO: 2 (or equivalent position of other albumins or variants or fragments thereof).

A variant albumin may comprise alterations at positions 345+573 of SEQ ID NO: 2 (or equivalent position of other albumins or variants or fragments thereof).

A variant albumin may comprise alterations at positions 349+573 of SEQ ID NO: 2 (or equivalent position of other albumins or variants or fragments thereof).

A variant albumin may comprise alterations at positions 381+573 of SEQ ID NO: 2 (or equivalent position of other albumins or variants or fragments thereof).

A variant albumin may comprise alterations at positions 384+573 of SEQ ID NO: 2 (or equivalent position of other albumins or variants or fragments thereof).

A variant albumin may comprise alterations at positions 83+342 of SEQ ID NO: 2 (or equivalent position of other albumins or variants or fragments thereof).

A variant albumin may comprise alterations at positions 83+345 of SEQ ID NO: 2 (or equivalent position of other albumins or variants or fragments thereof).

A variant albumin may comprise alterations at positions 83+349 of SEQ ID NO: 2 (or equivalent position of other albumins or variants or fragments thereof).

A variant albumin may comprise alterations at positions 83+381 of SEQ ID NO: 2 (or equivalent position of other albumins or variants or fragments thereof).

A variant albumin may comprise alterations at positions 83+384 of SEQ ID NO: 2 (or equivalent position of other albumins or variants or fragments thereof).

A variant albumin may comprise alterations at positions 83+342+573 of SEQ ID NO: 2 (or equivalent position of other albumins or variants or fragments thereof), e.g. T83N, K or S+S342Y, W, F, H, T, N, or Q, A, C, I, L, P, V or Y+K573P, Y or W, especially T83N+S342Y+K573P (SEQ ID NO: 103).

A variant albumin may comprise alterations at positions 83+345+573 of SEQ ID NO: 2 (or equivalent position of other albumins or variants or fragments thereof).

A variant albumin may comprise alterations at positions 83+349+573 of SEQ ID NO: 2 (or equivalent position of other albumins or variants or fragments thereof) e.g. T83N, K or S+L349F, W, Y, H, P, K or Q+K573P, Y or W, especially T83N+L349F+K573P (SEQ ID NO: 104).

A variant albumin may comprise alterations at positions 83+381+573 of SEQ ID NO: 2 (or equivalent position of other albumins or variants or fragments thereof), e.g. T83N, K or S+V381, G or A+K573P, Y or W, especially T83N+V381G+K573P (SEQ ID NO: 105).

A variant albumin may comprise alterations at positions 83+384+573 of SEQ ID NO: 2 (or equivalent position of other albumins or variants or fragments thereof).

Advantageously, the polypeptide retains substantially the same tertiary structure (or, for a fragment, the relevant part of the structure) as a reference or parent albumin such as HSA. The skilled person understand the term ‘substantially the same tertiary structure’ bearing in mind that some degree of variation in tertiary structure is expected as all proteins have some degree of structural flexibility. This applies particularly to polypeptides having a higher binding affinity to FcRn than the parent or reference albumin (e.g. HSA) has to FcRn.

One or more (several) of the His residues may or may not be maintained relative to the parent albumin. For example, with reference to SEQ ID NO: 2, one or more (several) of the following His residues may be maintained: 3, 9, 39, 67, 105, 128, 146, 242, 247, 288, 338, 367, 440, 464, 510, 535. One or more, preferably all, of the His residues in Domain I are maintained (i.e. 3, 9, 39, 67, 105, 128, 146). One or more, preferably all, of the His residues in Domain II are maintained (i.e. 242, 247, 288, 338, 367). One or more, preferably all, of the His residues in Domain III are maintained (i.e. 440, 464, 510, 535). One or more or all three of His 464, 510, 535 may be maintained.

It is preferred that at least 1, 2, 3, 4, 5, 6, 7, 8, 9, 10, 11, 12, 13, 14, 15, 16 or 17 of the disulphide bonds of the albumin are maintained in the polypeptide. For a polypeptide derived from a full length albumin, it is preferred that all disulphide bonds usually present in that albumin are maintained. For a polypeptide derived from a fragment of albumin, it is preferred that all disulphide bonds usually present in that fragment are maintained. It is preferred that Cys34 (or equivalent in other albumins e.g. non-human albumins) is maintained.

For all aspects of the invention fusion partner polypeptides and/or conjugates may comprise one or more (several) of: 4-1 BB ligand, 5-helix, A human C-C chemokine, A human L105 chemokine, A human L105 chemokine designated huL105_3, A monokine induced by gamma-interferon (MIG), A partial CXCR4B protein, A platelet basic protein (PBP), α1-antitrypsin, ACRP-30 Homologue; Complement Component C1q C, Adenoid-expressed chemokine (ADEC), aFGF; FGF-1, AGF, AGF Protein, albumin, an etoposide, angiostatin, Anthrax vaccine, Antibodies specific for collapsin, antistasin, Anti-TGF beta family antibodies, antithrombin III, APM-1; ACRP-30; Famoxin, apo-lipoprotein species, Arylsulfatase B, b57 Protein, BCMA, Beta-thromboglobulin protein (beta-TG), bFGF; FGF2, Blood coagulation factors, BMP Processing Enzyme Furin, BMP-10, BMP-12, BMP-15, BMP-17, BMP-18, BMP-2B, BMP-4, BMP-5, BMP-6, BMP-9, Bone Morphogenic Protein-2, calcitonin, Calpain-10a, Calpain-10b, Calpain-10c, Cancer Vaccine, Carboxypeptidase, C-C chemokine, MCP2, CCR5 variant, CCR7, CCR7, CD11a Mab, CD137; 4-1BB Receptor Protein, CD20 Mab, CD27, CD27L, CD30, CD30 ligand, CD33 immunotoxin, CD40, CD40L, CD52 Mab, Cerebus Protein, Chemokine Eotaxin, Chemokine hIL-8, Chemokine hMCP1, Chemokine hMCP1a, Chemokine hMCP1b, Chemokine hMCP2, Chemokine hMCP3, Chemokine hSDF1b, Chemokine MCP-4, chemokine TECK and TECK variant, Chemokine-like protein IL-8M1 Full-Length and Mature, Chemokine-like protein IL-8M10 Full-Length and Mature, Chemokine-like protein IL-8M3, Chemokine-like protein IL-8M8 Full-Length and Mature, Chemokine-like protein IL-8M9 Full-Length and Mature, Chemokine-like protein PF4-414 Full-Length and Mature, Chemokine-like protein PF4-426 Full-Length and Mature, Chemokine-like protein PF4-M2 Full-Length and Mature, Cholera vaccine, Chondromodulin-like protein, c-kit ligand; SCF; Mast cell growth factor; MGF; Fibrosarcoma-derived stem cell factor, CNTF and fragment thereof (such as CNTFAx15′(Axokine™)), coagulation factors in both pre and active forms, collagens, Complement C5 Mab, Connective tissue activating protein-Ill, CTAA16.88 Mab, CTAP-III, CTLA4-Ig, CTLA-8, CXC3, CXC3, CXCR3; CXC chemokine receptor 3, cyanovirin-N, Darbepoetin, designated exodus, designated huL105_7, DIL-40, DNase, EDAR, EGF Receptor Mab, ENA-78, Endostatin, Eotaxin, Epithelial neutrophil activating protein-78, EPO receptor; EPOR, erythropoietin (EPO) and EPO mimics, Eutropin, Exodus protein, Factor IX, Factor VII, Factor VIII, Factor X and Factor XIII, FAS Ligand Inhibitory Protein (DcR3), FasL, FasL, FasL, FGF, FGF-12; Fibroblast growth factor homologous factor-1, FGF-15, FGF-16, FGF-18, FGF-3; INT-2, FGF-4; gelonin, HST-1; HBGF-4, FGF-5, FGF-6; Heparin binding secreted transforming factor-2, FGF-8, FGF-9; Glia activating factor, fibrinogen, flt-1, flt-3 ligand, Follicle stimulating hormone Alpha subunit, Follicle stimulating hormone Beta subunit, Follitropin, Fractalkine, fragment. myofibrillar protein Troponin I, FSH, Galactosidase, Galectin-4, G-CSF, GDF-1, Gene therapy, Glioma-derived growth factor, glucagon, glucagon-like peptides, Glucocerebrosidase, glucose oxidase, Glucosidase, Glycodelin-A; Progesterone-associated endometrial protein, GM-CSF, gonadotropin, Granulocyte chemotactic protein-2 (GCP-2), Granulocyte-macrophage colony stimulating factor, growth hormone, Growth related oncogene-alpha (GRO-alpha), Growth related oncogene-beta (GRO-beta), Growth related oncogene-gamma (GRO-gamma), hAPO-4; TROY, hCG, Hepatitus B surface Antigen, Hepatitus B Vaccine, HER2 Receptor Mab, hirudin, HIV gp120, HIV gp41, HIV Inhibitor Peptide, HIV Inhibitor Peptide, HIV Inhibitor Peptide, HIV protease inhibiting peptides, HIV-1 protease inhibitors, HPV vaccine, Human 6CKine protein, Human Act-2 protein, Human adipogenesis inhibitory factor, human B cell stimulating factor-2 receptor, Human beta-chemokine H1305 (MCP-2), Human C-C chemokine DGWCC, Human CC chemokine ELC protein, Human CC type chemokine interleukin C, Human CCC3 protein, Human CCF18 chemokine, Human CC-type chemokine protein designated SLC (secondary lymphoid chemokine), Human chemokine beta-8 short forms, Human chemokine C10, Human chemokine CC-2, Human chemokine CC-3, Human chemokine CCR-2, Human chemokine Ckbeta-7, Human chemokine ENA-78, Human chemokine eotaxin, Human chemokine GRO alpha, Human chemokine GROalpha, Human chemokine GRObeta, Human chemokine HCC-1, Human chemokine HCC-1, Human chemokine 1-309, Human chemokine IP-10, Human chemokine L105_3, Human chemokine L105_7, Human chemokine MIG, Human chemokine MIG-beta protein, Human chemokine MIP-1alpha, Human chemokine MIP1beta, Human chemokine MIP-3alpha, Human chemokine MIP-3beta, Human chemokine PF4, Human chemokine protein 331D5, Human chemokine protein 61164, Human chemokine receptor CXCR3, Human chemokine SDF1alpha, Human chemokine SDF1beta, Human chemokine ZSIG-35, Human Chr19Kine protein, Human CKbeta-9, Human CKbeta-9, Human CX3C 111 amino acid chemokine, Human DNAX interleukin-40, Human DVic-1 C-C chemokine, Human EDIRF I protein sequence, Human EDIRF II protein sequence, Human eosinocyte CC type chemokine eotaxin, Human eosinophil-expressed chemokine (EEC), Human fast twitch skeletal muscle troponin C, Human fast twitch skeletal muscle troponin I, Human fast twitch skeletal muscle Troponin subunit C, Human fast twitch skeletal muscle Troponin subunit I Protein, Human fast twitch skeletal muscle Troponin subunit T, Human fast twitch skeletal muscle troponin T, Human foetal spleen expressed chemokine, FSEC, Human GM-CSF receptor, Human gro-alpha chemokine, Human gro-beta chemokine, Human gro-gamma chemokine, Human IL-16 protein, Human IL-1RD10 protein sequence, Human IL-1RD9, Human IL-5 receptor alpha chain, Human IL-6 receptor, Human IL-8 receptor protein hIL8RA, Human IL-8 receptor protein hIL8RB, Human IL-9 receptor protein, Human IL-9 receptor protein variant #3, Human IL-9 receptor protein variant fragment, Human IL-9 receptor protein variant fragment#3, Human interleukin 1 delta, Human Interleukin 10, Human Interleukin 10, Human interleukin 18, Human interleukin 18 derivatives, Human interleukin-1 beta precursor, Human interleukin-1 beta precursor, Human interleukin-1 receptor accessory protein, Human interleukin-1 receptor antagonist beta, Human interleukin-1 type-3 receptor, Human Interleukin-10 (precursor), Human Interleukin-10 (precursor), Human interleukin-11 receptor, Human interleukin-12 40 kD subunit, Human interleukin-12 beta-1 receptor, Human interleukin-12 beta-2 receptor, Human Interleukin-12 p35 protein, Human Interleukin-12 p40 protein, Human interleukin-12 receptor, Human interleukin-13 alpha receptor, Human interleukin-13 beta receptor, Human interleukin-15, Human interleukin-15 receptor from clone P1, Human interleukin-17 receptor, Human interleukin-18 protein (IL-18), Human interleukin-3, human interleukin-3 receptor, Human interleukin-3 variant, Human interleukin-4 receptor, Human interleukin-5, Human interleukin-6, Human interleukin-7, Human interleukin-7, Human interleukin-8 (IL-8), Human intracellular IL-1 receptor antagonist, Human IP-10 and HIV-1 gp120 hypervariable region fusion protein, Human IP-10 and human Muc-1 core epitope (VNT) fusion protein, human liver and activation regulated chemokine (LARC), Human Lkn-1 Full-Length and Mature protein, Human mammary associated chemokine (MACK) protein Full-Length and Mature, Human mature chemokine Ckbeta-7, Human mature gro-alpha, Human mature gro-gamma polypeptide used to treat sepsis, Human MCP-3 and human Muc-1 core epitope (VNT) fusion protein, Human MI10 protein, Human MI1A protein, Human monocyte chemoattractant factor hMCP-1, Human monocyte chemoattractant factor hMCP-3, Human monocyte chemotactic proprotein (MCPP) sequence, Human neurotactin chemokine like domain, Human non-ELR CXC chemokine H174, Human non-ELR CXC chemokine IP10, Human non-ELR CXC chemokine Mig, Human PAI-1 mutants, Human protein with IL-16 activity, Human protein with IL-16 activity, Human secondary lymphoid chemokine (SLC), Human SISD protein, Human STOP-1, Human stromal cell-derived chemokine, SDF-1, Human T cell mixed lymphocyte reaction expressed chemokine (TMEC), Human thymus and activation regulated cytokine (TARO), Human thymus expressed, Human TNF-alpha, Human TNF-alpha, Human TNF-beta (LT-alpha), Human type CC chemokine eotaxin 3 protein sequence, Human type II interleukin-1 receptor, Human wild-type interleukin-4 (hIL-4) protein, Human ZCHEMO-8 protein, Humanized Anti-VEGF Antibodies, and fragments thereof, Humanized Anti-VEGF Antibodies, and fragments thereof, Hyaluronidase, ICE 10 kD subunit, ICE 20 kD subunit, ICE 22 kD subunit, Iduronate-2-sulfatase, Iduronidase, IL-1 alpha, IL-1 beta, IL-1 inhibitor (IL-1i), IL-1 mature, IL-10 receptor, IL-11, IL-11, IL-12 p40 subunit, IL-13, IL-14, IL-15, IL-15 receptor, IL-17, IL-17 receptor, II-17 receptor, Il-17 receptor, IL-19, IL-1i fragments, IL1-receptor antagonist, IL-21 (TIF), IL-3 containing fusion protein, IL-3 mutant proteins, IL-3 variants, IL-3 variants, IL-4, IL-4 mutein, IL-4 mutein Y124G, IL-4 mutein Y124X, IL-4 muteins, Il-5 receptor, IL-6, Il-6 receptor, IL-7 receptor clone, IL-8 receptor, IL-9 mature protein variant (Met117 version), immunoglobulins or immunoglobulin-based molecules or fragment of either (e.g. a Small Modular ImmunoPharmaceutical™ (“SMIP”) or dAb, Fab′ fragments, F(ab′)2, scAb, scFv or scFv fragment), including but not limited to plasminogen, Influenza Vaccine, Inhibin alpha, Inhibin beta, insulin, insulin-like growth factor, Integrin Mab, inter-alpha trypsin inhibitor, inter-alpha trypsin inhibitor, Interferon gamma-inducible protein (IP-10), interferons (such as interferon alpha species and sub-species, interferon beta species and sub-species, interferon gamma species and sub-species), interferons (such as interferon alpha species and sub-species, interferon beta species and sub-species, interferon gamma species and sub-species), Interleukin 6, Interleukin 8 (IL-8) receptor, Interleukin 8 receptor B, Interleukin-1alpha, Interleukin-2 receptor associated protein p43, interleukin-3, interleukin-4 muteins, Interleukin-8 (IL-8) protein, interleukin-9, Interleukin-9 (IL-9) mature protein (Thr117 version), interleukins (such as IL10, IL11 and IL2), interleukins (such as IL10, IL11 and IL2), Japanese encephalitis vaccine, Kalikrein Inhibitor, Keratinocyte growth factor, Kunitz domain protein (such as aprotinin, amyloid precursor protein and those described in WO 03/066824, with or without albumin fusions), Kunitz domain protein (such as aprotinin, amyloid precursor protein and those described in WO 03/066824, with or without albumin fusions), LACI, lactoferrin, Latent TGF-beta binding protein II, leptin, Liver expressed chemokine-1 (LVEC-1), Liver expressed chemokine-2 (LVEC-2), LT-alpha, LT-beta, Luteinization Hormone, Lyme Vaccine, Lymphotactin, Macrophage derived chemokine analogue MDC (n+1), Macrophage derived chemokine analogue MDC-eyfy, Macrophage derived chemokine analogue MDC-yl, Macrophage derived chemokine, MDC, Macrophage-derived chemokine (MDC), Maspin; Protease Inhibitor 5, MCP-1 receptor, MCP-1a, MCP-1b, MCP-3, MCP-4 receptor, M-CSF, Melanoma inhibiting protein, Membrane-bound proteins, Met117 human interleukin 9, MIP-3 alpha, MIP-3 beta, MIP-Gamma, MIRAP, Modified Rantes, monoclonal antibody, MP52, Mutant Interleukin 6 S176R, myofibrillar contractile protein Troponin I, Natriuretic Peptide, Nerve Growth Factor-beta, Nerve Growth Factor-beta2, Neuropilin-1, Neuropilin-2, Neurotactin, Neurotrophin-3, Neurotrophin-4, Neurotrophin-4a, Neurotrophin-4b, Neurotrophin-4c, Neurotrophin-4d, Neutrophil activating peptide-2 (NAP-2), NOGO-66 Receptor, NOGO-A, NOGO-B, NOGO-C, Novel beta-chemokine designated PTEC, N-terminal modified chemokine GroHEK/hSDF-1alpha, N-terminal modified chemokine GroHEK/hSDF-1beta, N-terminal modified chemokine met-hSDF-1 alpha, N-terminal modified chemokine met-hSDF-1 beta, OPGL, Osteogenic Protein-1; OP-1; BMP-7, Osteogenic Protein-2, OX40; ACT-4, OX40L, Oxytocin (Neurophysin I), parathyroid hormone, Patched, Patched-2, PDGF-D, Pertussis toxoid, Pituitary expressed chemokine (PGEC), Placental Growth Factor, Placental Growth Factor-2, Plasminogen Activator Inhibitor-1; PAI-1, Plasminogen Activator Inhibitor-2; PAI-2, Plasminogen Activator Inhibitor-2; PAI-2, Platelet derived growth factor, Platelet derived growth factor Bv-sis, Platelet derived growth factor precursor A, Platelet derived growth factor precursor B, Platelet Mab, platelet-derived endothelial cell growth factor (PD-ECGF), Platelet-Derived Growth Factor A chain, Platelet-Derived Growth Factor B chain, polypeptide used to treat sepsis, Preproapolipoprotein “milano” variant, Preproapolipoprotein “paris” variant, pre-thrombin, Primate CC chemokine “ILINCK”, Primate CXC chemokine “IBICK”, proinsulin, Prolactin, Prolactin2, prosaptide, Protease inhibitor peptides, Protein C, Protein S, pro-thrombin, prourokinase, RANTES, RANTES 8-68, RANTES 9-68, RANTES peptide, RANTES receptor, Recombinant interleukin-16, Resistin, restrictocin, Retroviral protease inhibitors, ricin, Rotavirus Vaccine, RSV Mab, saporin, sarcin, Secreted and Transmembrane polypeptides, Secreted and Transmembrane polypeptides, serum cholinesterase, serum protein (such as a blood clotting factor), Soluble BMP Receptor Kinase Protein-3, Soluble VEGF Receptor, Stem Cell Inhibitory Factor, Straphylococcus Vaccine, Stromal Derived Factor-1 alpha, Stromal Derived Factor-1 beta, Substance P (tachykinin), T1249 peptide, T20 peptide, T4 Endonuclease, TACI, Tarc, TGF-beta 1, TGF-beta 2, Thr117 human interleukin 9, thrombin, thrombopoietin, Thrombopoietin derivative1, Thrombopoietin derivative2, Thrombopoietin derivative3, Thrombopoietin derivative4, Thrombopoietin derivative5, Thrombopoietin derivative6, Thrombopoietin derivative7, Thymus expressed chemokine (TECK), Thyroid stimulating Hormone, tick anticoagulant peptide, Tim-1 protein, TNF-alpha precursor, TNF-R, TNF-RII; TNF p75 Receptor; Death Receptor, tPA, transferrin, transforming growth factor beta, Troponin peptides, Truncated monocyte chemotactic protein 2 (6-76), Truncated monocyte chemotactic protein 2 (6-76), Truncated RANTES protein (3-68), tumour necrosis factor, Urate Oxidase, urokinase, Vasopressin (Neurophysin II), VEGF R-3; flt-4, VEGF Receptor; KDR; flk-1, VEGF-110, VEGF-121, VEGF-138, VEGF-145, VEGF-162, VEGF-165, VEGF-182, VEGF-189, VEGF-206, VEGF-D, VEGF-E; VEGF-X, von Willebrand's factor, Wild type monocyte chemotactic protein 2, Wild type monocyte chemotactic protein 2, ZTGF-beta 9, alternative antibody scaffolds e.g. anticalin(s), adnectin(s), fibrinogen fragment(s), nanobodies such as camelid nanobodies, infestin, and/or any of the molecules mentioned in WO01/79271 (particularly page 9 and/or Table 1), WO 2003/59934 (particularly Table 1), WO03/060071 (particularly Table 1) or WO01/079480 (particularly Table 1) (each incorporated herein by reference in their entirety).

Furthermore, conjugates may comprise one or more (several) of chemotherapy drugs such as: 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, A, Abraxane, Accutane®, Actinomycin-D, Adriamycin®, Adrucil®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin®, Arranon®, Arsenic Trioxide, Asparaginase, ATRA, Avastin®, Azacitidine, BCG, BCNU, Bevacizumab, Bexarotene, BEXXAR®, Bicalutamide, BiCNU, Blenoxane®, Bleomycin, Bortezomib, Busulfan, Busulfex®, C225, Calcium Leucovorin, Campath®, Camptosar®, Camptothecin-11, Capecitabine, Carac™, Carboplatin, Carmustine, Carmustine Wafer, Casodex®, CC-5013, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine, Cytarabine Liposomal, Cytosar-U®, Cytoxan®, Dacarbazine, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin diftitox, DepoCyt™, Dexamethasone, Dexamethasone acetate, Dexamethasone Sodium Phosphate, Dexasone, Dexrazoxane, DHAD, DIC, Diodex, Docetaxel, Doxil®, Doxorubicin, Doxorubicin liposomal, Droxia™, DTIC, DTIC-Dome®, Duralone®, Efudex®, Eligard™, Ellence™, Eloxatin™, Elspar®, Emcyt®, Epirubicin, Epoetin alfa, Erbitux™, Erlotinib, Erwinia L-asparaginase, Estramustine, Ethyol, Etopophos®, Etoposide, Etoposide Phosphate, Eulexin®, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, G-CSF, Gefitinib, Gemcitabine, Gemtuzumab ozogamicin, Gemzar®, Gleevec™, Gliadel® Wafer, GM-CSF, Goserelin, Granulocyte—Colony Stimulating Factor, Granulocyte Macrophage Colony Stimulating Factor, Halotestin®, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetan, Idamycin®, Idarubicin, Ifex®, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A® (interferon alfa-2b), Iressa®, Irinotecan, Isotretinoin, Kidrolase®, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, M, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar®, Neulasta™, Neumega®, Neupogen®, Nexavar®, Nilandron®, Nilutamide, Nipent®, Nitrogen Mustard, Novaldex®, Novantrone®, Octreotide, Octreotide acetate, Oncospar®, Oncovin®, Ontak®, Onxal™, Oprevelkin, Orapred®, Orasone®, Oxaliplatin, a taxol or taxol derivative e.g. Paclitaxel or Paclitaxel Protein-bound, Pamidronate, Panitumumab, Panretin®, Paraplatin®, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, R, Raloxifene, Revlimid®, Rheumatrex®, Rituxan®, Rituximab, Roferon-A® (Interferon Alfa-2a), Rubex®, Rubidomycin hydrochloride, Sandostatin®, Sandostatin LAR®, Sargramostim, Solu-Cortef®, Solu-Medrol®, Sorafenib, SPRYCEL™, STI-571, Streptozocin, SU11248, Sunitinib, Sutent®, Tamoxifen, Tarceva®, Targretin®, Taxol®, Taxotere®, Temodar®, Temozolomide, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Tositumomab, Trastuzumab, Tretinoin, Trexall™, Trisenox®, TSPA, TYKERB®, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, VP-16, Vumon®, Xeloda®, Zanosar®, Zevalin™, Zinecard®, Zoladex®, Zoledronic acid, Zolinza, Zometa®; radiopharmaceuticals such as: Carbon-11, Carbon-14, Chromium-51, Cobalt-57, Cobalt-58, Erbium-169, Fluorine-18, Gallium-67, Gold-198, Indium-111, Indium-113m, Iodine-123, Iodine-125, Iodine-131, Iron-59, Krypton-81m, Nitrogen-13, Oxygen-15, Phosphorous-32, Rhenium-186, Rubidium-82, Samarium-153, Selenium-75, Strontium-89, Technetium-99m, Thallium-201, Tritium, Xenon-127, Xenon-133, Yttrium-90; imaging agents such as Gadolinium, magnetite, manganese, technetium, I125, I131, P32, TI201, Iopamidol, PET-FDG.

Further fusion partners, conjugation partners and/or molecules for inclusion in a nanoparticle, associate or composition according to the invention include: acromegaly drugs e.g. somatuline, lanreotide, octreotide, Sandostatin; antithrombotics e.g. bivalirudin, Angiomax, dalteparin, Fragmin, enoxaparin, Lovenox, Drotrecogin alfa (e.g. Activated), Xigris, heparin; assisted reproductive therapy compounds e.g. choriogonadotropin, Ovidrel, follitropin, alpha/beta; enzymes e.g. hyaluronidase, Hylenex; diabetes drugs e.g. exenatide, Byetta, glucagon, insulin, liraglutide, albiglutide, GLP-1 agonists, exendin or an exendin analog; compounds useful in diagnosis e.g. protirelin, Thyrel TRH Thypinone, secretin (e.g. synthetic human), Chirhostim, thyrotropin (e.g. alpha), Thyrogen' erythropoiesis drugs e.g. Darbepoetin alfa, Aranesp, Epoetin alfa, Epogen, Eprex, drugs for the treatment of genetic defects e.g. pegademase, drugs for the treatment of growth failure e.g. Adagen, mecasermin, rinfabate, drugs for the treatment of cystic fibrosis e.g. Dornase alfa, Pulmozyme, drugs for the treatment of metaoblic disorders e.g. Agalsidase beta, Fabrazyme, alglucosidase alpha, Myozyme, Laronidase, Aldurazyme, drugs for the treatment of genital wart intralesional e.g. Interferon alfa-n3, Alferon N, drugs for the treatment of granulomatous disease e.g. Interferon gamma-1b, Actimmune; drugs for the treatment of growth failure e.g. pegvisomant, Somavert, somatropin, Genotropin, Nutropin, Humatrope, Serostim, Protropin; drugs for the treatment of heart failure e.g. nesiritide, Natrecor; drugs for the treatment of hemophilia e.g. a coagulation factor e.g. Factor VIII, Helixate FS, Kogenate FS, Factor IX, BeneFIX, Factor VIIa, Novoseven, desmopressin, Stimate, DDAVP; hemopoetic drugs e.g. Filgrastim (G-CSF), Neupogen, Oprelvekin, Neumega, Pegfilgrastim, Neulasta, Sargramostim, Leukine; drugs for the treatment of hepatitis C e.g. Interferon alfa-2a, Roferon A, Interferon alfa-2b, Intron A, Interferon alfacon-1, Infergen, Peginterferon alfa-2a, Pegasys, Peginterferon alfa-2b, PEG-Intron; drugs for the treatment of HIV e.g. enfuvirtide, Fuzeon; Fabs e.g. Fab (antithrombin), Abciximab, ReoPro; monoclonal antibodies e.g. Daclizumab, Zenapax; antiviral monoclonal antibodies e.g. Palivizumab, Synagis; monoclonal antibodies for the treatment of asthma e.g. Omalizumab, Xolair; monoclonal antibodies for use in diagnostic imaging e.g. Arcitumomab, CEA-Scan, Capromab Pendetide, ProstaScint, Satumomab Pendetide, OncoScint CR/OV, Fabs for use in diagnostic imaging e.g. Nofetumomab, Verluma; iimmuno-supressant monoclonal antibodies e.g. Basiliximab, Simulect, Muromonab-CD3, Orthoclone OKT3; monoclonal antibodies for the treatment of malignancy e.g. Alemtuzumab, Campath, Ibritumomab tiuxetan, Zevalin, Rituximab, Rituxan, Trastuzumab, Herceptin; monoclonal antibodies for the treatment of rheumatoid arthritis (RA) e.g. Adalimumab, Humira, Infliximab, Remicade; monoclonal antibodies for use as a radio-immuno-therapeutic e.g. Tositumomab and Iodine I¹³¹, Tositumomab, Bexxar; drugs for the treatment of macular degeneration e.g. pegaptanib, Macugen; drugs for the treatment of malignancy e.g. Aldesleukin, Proleukin, Interleukin-2, Asparaginase, Elspar, Rasburicase, Elitek, Denileukin diftitox, Ontak, Pegaspargase, Oncaspar, goserelin, leuprolide; drugs for the treatment of multiple sclerosis (MS) e.g. Glatiramer acetate (e.g. copolymer-1), Copaxone, Interferon beta-1a, Avonex, Interferon beta-1a, Rebif, Interferon beta-1 b, Betaseron; drugs for the treatment of mucositis e.g. palifermin, Kepivance; drug for the treatment of dystonia e.g., neurotoxin, Botulinum Toxin Type A, BOTOX, BOTOX Cosmetic, Botulinum Toxin Type B, MYOBLOC; drugs for the treatment of osteoporosis e.g. teriparatide, Forteo; drugs for the treatment of psoriasis e.g. Alefacept, Amevive; drugs for the treatment of RA e.g. abatacept, Orencia, Anakinra, Kineret, Etanercept, Enbrel; thrombolytics e.g. Alteplase, Activase, rtPA, Anistreplase, Eminase, Reteplase, Retavase, Streptokinase, Streptase, Tenecteplase, TNKase, Urokinase, Abbokinase, Kinlytic; drugs for the treatment of osteoporosis e.g. calcitonin (e.g. salmon), Miacalcin, Fortical, drugs for the treatment of skin ulcers e.g. Becaplermin, Regranex, Collagenase, Santyl.

Such polypeptides and chemical compounds may be referred to as diagnostic moieties, therapeutic moieties, prophylactic moieties or beneficial moieties.

Preferably the fusion partner and/or conjugation partner is not an albumin, variant or fragment thereof.

One or more (several) therapeutic or prophylactic polypeptides may be fused to the N-terminus, the C-terminus of albumin, inserted into a loop in the albumin structure or any combination thereof. It may or it may not comprise linker sequences separating the various components of the fusion polypeptide.

Teachings relating to fusions of albumin or a fragment thereof are known in the art and the skilled person will appreciate that such teachings can also be applied to the invention. WO 2001/79271A and WO 2003/59934 (incorporated herein by reference) also contain examples of therapeutic and prophylactic polypeptides that may be fused to albumin or fragments thereof, and these examples apply also to the invention.

Embodiments of the present invention in include:

1. A polypeptide which is a variant of a parent albumin, fragment thereof or fusion polypeptide comprising said variant albumin or a fragment thereof having an altered binding affinity to FcRn compared with the binding affinity of a parent albumin, reference albumin, fragment thereof or fusion polypeptide comprising said parent albumin, reference albumin or fragment or fusion thereof to FcRn, wherein the polypeptide comprises one or more (several) alterations in Domain II of albumin selected from the group consisting of positions corresponding to positions 349, 342, 381, 345, 384, 198, 206, 340, 341, 343, 344, 352, 382, 348, and/or 383 in SEQ ID NO: 2, wherein the polypeptide does not consist of SEQ ID NO: 2 with alteration E382K.
2. The polypeptide according to embodiment 1 wherein the alteration at the position corresponding to position 349, 342, 381, 345, 384, 198, 206, 340, 341, 343, 344, 352, 382, 348, and/or 383 is a substitution.
3. The polypeptide according to embodiment 2 wherein the substitution at the position corresponding to position 349 is to F, W Y H P, K or Q, preferably F.
4. The polypeptide according to embodiment 2 or 3 wherein the substitution at the position corresponding to position 342 is to Y, W, F, H, T, N, Q, A, C, I, L, P, V, preferably Y.
5. The polypeptide according to any of embodiments 2 to 4 wherein the substitution at the position corresponding to position 381 is to G or A, preferably G.
6. The polypeptide according to any of embodiments 2 to 5 wherein the substitution at the position corresponding to position 345 is to E, H, I or Q.
7. The polypeptide according to any of embodiments 1 to 6 further comprising a substitution at the position corresponding to position 573.
8. The polypeptide according to embodiment 7 wherein the substitution at the position corresponding to position 573 is to P, Y or W.
9. The polypeptide according to any of embodiments 1 to 8 further comprising a substitution at the position corresponding to position 83.
10. The polypeptide according to embodiment 9 wherein the substitution at the position corresponding to position 83 is to N, K or S.
11. The polypeptide according to any of embodiments 1 to 10 comprising substitutions at positions corresponding to positions 83, 342 and 573 (or equivalent position of other albumins or variants or fragments thereof), preferably T83N, K or S+S342Y, W, F, H, T, N, or Q, A, C, I, L, P, V or Y+K573P, Y or W, more preferably T83N+S342Y+K573P (SEQ ID NO: 103).
12. The polypeptide according to any of embodiments 1 to 11 comprising substitutions at positions corresponding to positions 83, 349 and 573 of SEQ ID NO: 2 (or equivalent position of other albumins or variants or fragments thereof), preferably T83N, K or S+L349F, W, Y, H, P, K or Q+K573P, Y or W, more preferably T83N+L349F+K573P (SEQ ID NO: 104).
13. The polypeptide according to any of embodiments 1 to 12 comprising substitutions at positions corresponding to positions 83, 381, and 573 of SEQ ID NO: 2 (or equivalent position of other albumins or variants or fragments thereof), preferably T83N, K or S+V381, G or A+K573P, Y or W, more preferably T83N+V381 G+K573P (SEQ ID NO: 105).
14. The polypeptide according to any of embodiments 1 to 13 wherein the parent albumin or reference albumin is HSA (SEQ ID NO: 2) or a fragment thereof, or a fusion polypeptide comprising HSA or a fragment thereof, most preferably SEQ ID NO: 2.
15. The polypeptide according to any of embodiments 1 to 14, having:
(a) a stronger binding affinity to FcRn and/or longer plasma half-life than a parent albumin, reference albumin, fragment thereof or fusion polypeptide comprising said parent albumin, reference albumin or fragment or fusion thereof; or
(b) a weaker binding affinity to FcRn and/or weaker plasma half-life than a parent albumin, reference albumin, fragment thereof or fusion polypeptide comprising said parent albumin, reference albumin or fragment or fusion thereof.
16. The polypeptide according any of embodiments 1 to 15, wherein the sequence identity of the polypeptide to SEQ ID NO: 2 is more than 80%, preferably more than 90%, more preferred more than 95%, more preferred more than 96%, even more preferred more than 97%, more preferred more than 98% and most preferred more than 99%.
17. A fusion polypeptide comprising a polypeptide according to any of embodiments 1 to 16 and a fusion partner polypeptide selected from a therapeutic, prophylactic, diagnostic, imaging or other beneficial polypeptide.
18. A method for preparing a polypeptide which is a variant of albumin, fragment thereof or fusion polypeptide comprising said variant albumin or fragment thereof having a binding affinity to FcRn which is altered compared to the binding affinity of a reference albumin, fragment or fusion thereof to FcRn, comprising:
(a) providing a nucleic acid encoding a parent albumin having at least 80% sequence identity to SEQ ID NO: 2;
(b) modifying the sequence of step (a), to encode a polypeptide which is a variant albumin, fragment thereof or fusion polypeptide comprising said variant albumin or fragment thereof comprising one or more (several) alterations in Domain II of albumin;
(c) introducing the modified sequence of step (b) in a suitable host cell;
(d) growing the cells in a suitable growth medium under condition leading to expression of the polypeptide; and
(e) recovering the polypeptide from the growth medium;
wherein the polypeptide has an altered binding affinity to FcRn and/or an altered plasma half-life compared with the half-life of a parent albumin, reference albumin, fragment thereof or fusion polypeptide comprising said parent albumin, reference albumin or fragment or fusion thereof.
   19. The method according to embodiment 18 wherein the one or more (several) alterations in Domain II of albumin are selected from the positions selected from the group consisting of positions corresponding to 349, 342, 381, 345, 384, 198, 206, 340, 341, 343, 344, 352, 382, 348, and/or 383 in SEQ ID NO: 2, wherein the polypeptide does not consist of SEQ ID NO: 2 with alteration E382K.
   20. The method according to embodiment 19 wherein the alteration at the position corresponding to position 349, 342, 381, 345, 384, 198, 206, 340, 341, 343, 344, 352, 382, 348, and/or 383 is a substitution.
   21. The method according to embodiment 20 wherein the substitution at the position corresponding to position 349 is to F, W Y H P, K or Q, preferably F.
   22. The method according to embodiment 20 or 21 wherein the substitution at the position corresponding to position 342 is to Y, W, F, H, T, N, Q, A, C, I, L, P, V, preferably Y.
   23. The method according to any of embodiments 20 to 22 wherein the substitution at the position corresponding to position 381 is to G or A, preferably G.
   24. The method according to any of embodiments 20 to 23 wherein the substitution at the position corresponding to position 345 is to E, H, I or Q.
   25. The method according to any of embodiments 19 to 24 further comprising a substitution at the position corresponding to position 573.
   26. The method according to embodiment 25 wherein the substitution at the position corresponding to position 573 is to P, Y or W.
   27. The method according to any of embodiments 19 to 26 further comprising a substitution at the position corresponding to position 83.
   28. The method according to embodiment 27 wherein the substitution at the position corresponding to position 83 is to N, K or S.
   29. The method according to any of embodiments 18 to 28 wherein the parent albumin is HSA (SEQ ID NO: 2) or a fragment thereof, or a fusion polypeptide comprising HSA or a fragment thereof, most preferably SEQ ID NO: 2.
   30. The method according any of embodiments 18 to 29, wherein the sequence identity of the polypeptide to SEQ ID NO: 2 is more than 80%, preferably more than 90%, more preferred more than 95%, more preferred more than 96%, even more preferred more than 97%, more preferred more than 98% and most preferred more than 99%.
   31. A conjugate comprising:
   a polypeptide according to any of embodiments 1 to 17;
   a polypeptide obtainable by a method according to any of embodiments 18 to 30; and
   a conjugation partner.
   32. The conjugate according to embodiment 31 wherein the conjugation partner is a therapeutic, prophylactic, diagnostic, imaging or other beneficial moiety.
   33. An associate comprising a polypeptide according to any of embodiments 1 to 17 or obtainable by a method according to any of embodiments 18 to 30 and a therapeutic, prophylactic, diagnostic, imaging or other beneficial moiety.
   34. A nanoparticle or microparticle comprising a polypeptide according to any of embodiments 1 to 17 or obtainable by a method according to any of embodiments 18 to 30, a conjugate according to embodiment 31 or 32 or an associate according to embodiment 33.
   35. A composition comprising a polypeptide according to any of embodiments 1 to 17 or obtainable by a method according to any of embodiments 18 to 30, a conjugate according to embodiment 31 or 32, an associate according to embodiment 33 or a nanoparticle or microparticle according embodiment 34, wherein the binding affinity of the polypeptide, fusion polypeptide, conjugate, associate or nanoparticle or microparticle to FcRn is:
   (a) stronger than the binding affinity of a composition comprising the corresponding parent albumin, reference albumin, fragment thereof or fusion polypeptide, conjugate, associate or nanoparticle or microparticle comprising said parent albumin, reference albumin or fragment or fusion thereof to FcRn; or
   (b) weaker than the binding affinity of a composition comprising the corresponding parent albumin, reference albumin, fragment thereof or fusion polypeptide, conjugate, associate or nanoparticle or microparticle comprising said parent albumin, reference albumin or fragment or fusion thereof to FcRn.
   36. A composition according to embodiment 35 where the binding affinity of the polypeptide, fusion polypeptide, conjugate, associate or nanoparticle or microparticle to FcRn is:
   (a) stronger than the binding affinity of a reference composition comprising or consisting of HSA (SEQ ID NO: 2) or a fragment, fusion, conjugate, associate, nanoparticle or microparticle thereof to FcRn; or
   (b) weaker than the binding affinity of a reference composition comprising or consisting of HSA (SEQ ID NO: 2) or a fragment, fusion, conjugate, associate, nanoparticle or microparticle thereof to FcRn.
   37. A composition according to embodiment 35 or 36, wherein the binding affinity of the variant to the polypeptide, fusion polypeptide, conjugate, associate or nanoparticle or microparticle to FcRn is less than 0.9×KD of the binding affinity of HSA to FcRn, more preferred less than 0.5×KD of HSA to FcRn, more preferred less than 0.1×KD of HSA to FcRn, even more preferred less than 0.05×KD of HSA to FcRn, even more preferred less than 0.02×KD of HSA to FcRn and most preferred less than 0.01×KD of a reference comprising or consisting of HSA (SEQ ID NO: 2) or a fragment, fusion, conjugate, associate, nanoparticle or microparticle thereof to FcRn.
   38. The composition according to any of embodiments 35 to 37, comprising a polypeptide according to any of embodiments 1 to 17 or obtainable by a method according to any of embodiments 18 to 30, a conjugate according to embodiment 31 or 32, an associate according to embodiment 33 or a nanoparticle or microparticle according embodiment 34, further comprising a compound comprising an antibody binding domain (ABD) and a therapeutic, prophylactic, diagnostic, imaging or other beneficial moiety.
   39. The composition according to any of embodiments 35 to 38, comprising a pharmaceutically acceptable carrier or excipient.
   40. Use of a polypeptide according to any of embodiments 1 to 17 or obtainable by a method according to any of embodiments 18 to 30, a conjugate according to embodiment 31 or 32, an associate according to embodiment 33 or a nanoparticle or microparticle according embodiment 34 or a composition according to any of embodiments 35 to 39 to alter the binding affinity to FcRn or half-life, preferably in plasma, of a therapeutic, prophylactic, diagnostic, imaging or other beneficial moiety.
   41. The use according to embodiment 40 wherein the binding affinity to FcRn is increased relative to the binding affinity of a reference comprising or consisting of HSA (SEQ ID NO: 2) or a fragment, fusion, conjugate, associate, nanoparticle or microparticle thereof to FcRn.
   42. The use according to embodiment 40 wherein the binding affinity to FcRn is decreased relative to the binding affinity of a reference comprising or consisting of HSA (SEQ ID NO: 2) or a fragment, fusion, conjugate, associate, nanoparticle or microparticle thereof to FcRn.
   43. A method for altering the binding affinity to FcRn or half-life preferably in plasma, of a molecule comprising:

(a) where the molecule is a polypeptide, fusing or conjugating the molecule to a polypeptide according to any of embodiments 1 to 17 or obtainable by a method of embodiments 18 to 30, or to a conjugate according to embodiment 31 or 32; associating the molecule to a polypeptide according to any of embodiments 1 to 17 or obtainable by a method of embodiments 18 to 30 or to a conjugate according to embodiment 31 or 32; incorporating the molecule in an associate according to embodiment 33, in nanoparticle or microparticle according to embodiment 34 or a composition according to any of embodiments 35 to 39;

(b) where the molecule is not a polypeptide, conjugating the molecule to a polypeptide according to any of embodiments 1 to 17 or obtainable by a method of embodiments 18 to 30, or to a conjugate according to embodiment 31 or 32; associating the molecule to a polypeptide according to any of embodiments 1 to 17 or obtainable by a method of embodiments 18 to 30 or to a conjugate according to embodiment 31 or 32; incorporating the molecule in an associate according to embodiment 33, in nanoparticle or microparticle according to embodiment 34 or a composition according to any of embodiments 35 to 39.

44. A method according to embodiment 43 wherein the molecule is a therapeutic, prophylactic, diagnostic, imaging or other beneficial moiety.
45. A polypeptide, fusion polypeptide, conjugate, associate, nanoparticle or microparticle or composition thereof according to any of embodiments 1 to 17, 31 to 39 or obtainable by the method of embodiments 18 to 30 wherein the polypeptide, fusion polypeptide, conjugate, associate, nanoparticle or microparticle or composition comprises one or more (several) moieties selected from those described herein.
46. A nucleic acid encoding the polypeptide or fusion polypeptide of any of embodiments 1 to 17.
47. A vector comprising a nucleic acid according to embodiment 46.
48. A host cell comprising a nucleic acid according to embodiment 46 or a vector according to embodiment 47.
49. A host cell according to embodiment 48 wherein the host cell is a eukaryote, preferably a yeast (such as Saccharomyces cerevisiae) or a mammalian cell (such as CHO or HEK) or a plant cell (such as rice).
50. A method of prophylaxis, treatment or diagnosis comprising administering a polypeptide, fusion polypeptide, conjugate, composition, associate, nanoparticle or microparticle or polynucleotide according to any of embodiments 1 to 17 or 31 to 39 or obtainable by the method of any of embodiments 18 to 30 to a subject.

The invention is further described by the following examples that should not be construed as limiting the scope of the invention.

### EXAMPLES

### Example 1

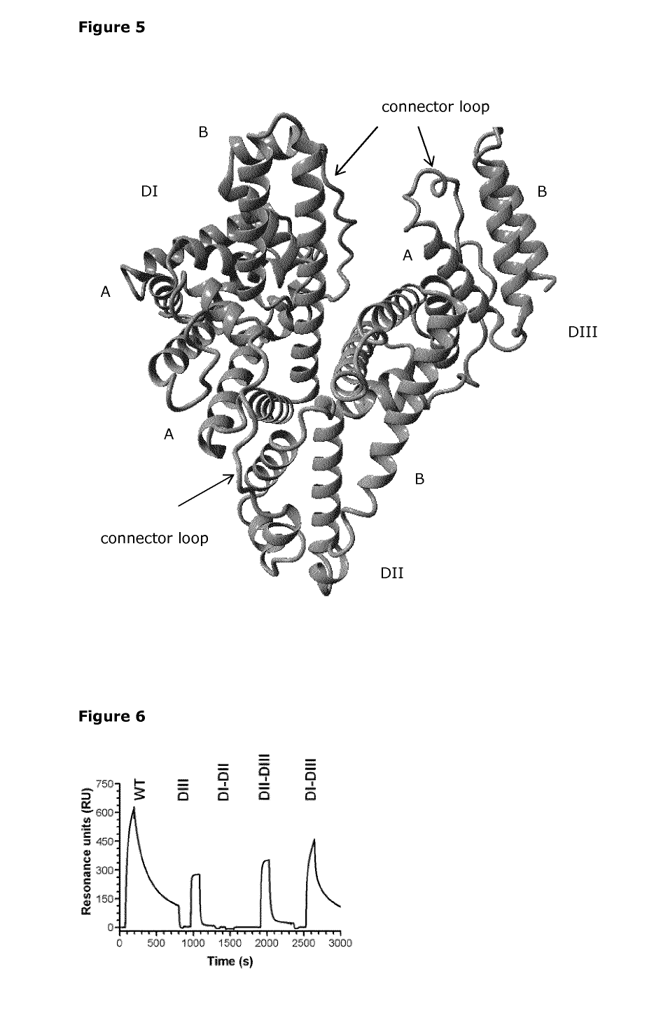

HSA comprises three structurally homologous domains, with a root-mean-square deviation (RMSD) of 3.78 Å (Angstrom) over the three domains, with the greatest similarity in shape observed between Domains II and III (Sugio et al., 1999, Protein Eng. 1999 June; 12(6):439-46.). All three domains contain sub-domains A and B, containing six and four alpha helices, respectively, connected by an extended loop (FIG. 5). Analysis of domain variants of HSA (DIII (SEQ ID NO: 27), DI-DII (SEQ ID NO: 97), DII-DIII (SEQ ID NO: 25), and DI-DIII (SEQ ID NO: 24)) in interaction studies with shFcRn identified Domain III as the principal binding region, whereby Domain III alone is capable of binding shFcRn in contrast to the DI-DII construct lacking Domain III, which was not proficient for binding (FIG. 6 and Andersen et al., (2012) Nature Communications 3, 610). While Domain II did not appear to impact on the binding of the DII-DIII construct, a DI-DIII construct (comprising residues 1-194 and 381-585 of SEQ ID NO: 2) showed improved binding compared to Domain III alone and, moreover, a sensorgram (provided as FIG. 6 of the present application) showed that association and dissociation of the DI-DIII constructed resembled that of WT HSA.

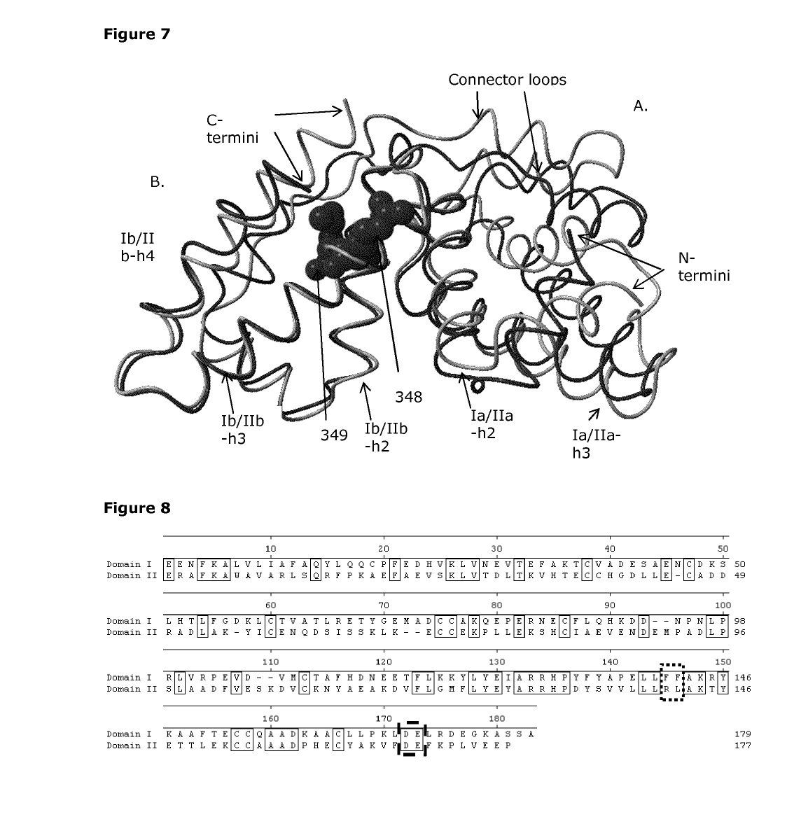

Hypothesising that Domain I has a positive impact on shFcRn binding when in the position of Domain II, as would be the case in the DI-DIII domain variant, the present invention provides a structural and sequence comparison which was used to design candidate variants postulated to have a modulating effect on the HSA:shFcRn interaction. Superposition was performed using YASARA version 12.1.19 (YASARA Bioscience, Graz, Austria; http://www.yasara.org/) and structures aligned using the MUSTANG module on default settings Graphical representations were done with the YASARA software. Superposition of Domain I (residues 1-194) onto Domain II (residues 195-380) yields an RMSD of 1.42 Å (Angstrom) over 112 aligned residues (FIG. 7). Co-ordinates for the crystal structure of HSA (accession number 1E78.pdb) were retrieved from the RCSB Protein Data Bank website and chain A used to produce the Domain I and II molecules for superposition. The N-terminal helix (about 15 residues) of HSA has no structural equivalent in Domain II and therefore sequence alignment was confined to regions with equivalent secondary structural elements (FIG. 8), although relative disposition of the helices does vary between the two domains. The position of the disulphide bonds within domains was taken into consideration with manual adjustment of the alignment. Agreement between Cα (Cₐₗₚₕₐ) atoms is good between helices Ia/IIa-h2 and Ia/IIa-h3 (residues 16-56 and 206-247, as designated by Sugio et al., 1999). The disposition of the secondary structural elements then varies until the start of the connector loop (residues 105 and 292). There is broad agreement between the loop regions, with some differences in helical turns. Agreement is good between Cα (Cₐₗₚₕₐ) residues from IbH1 to the final helices of each domain (I/IIb-h4), where the orientation of the helices differs.

Structural and sequence pairwise comparison was used to identify example positions where changes in the amino acid character occurred between Domain I and II, with such differences in character shown in FIG. 3. Examples of this analysis are shown in FIGS. 8 and 9, whereby aliphatic and basic residues in Domain II are substituted with the equivalent aromatic residues from Domain I, resulting in variants R348F and L349F.

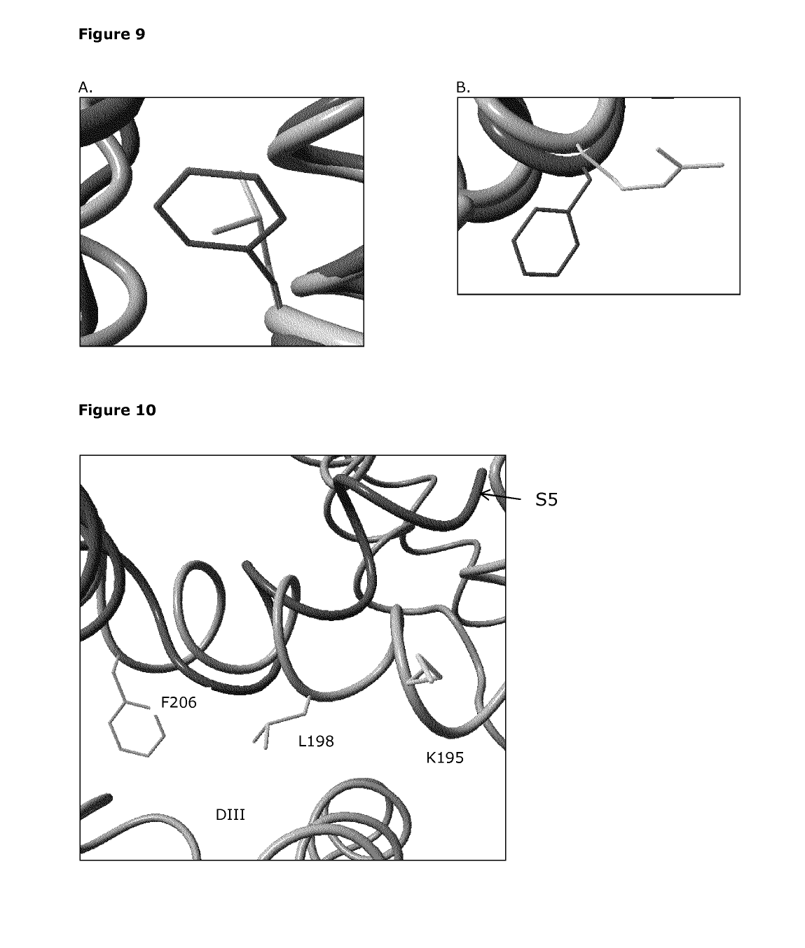

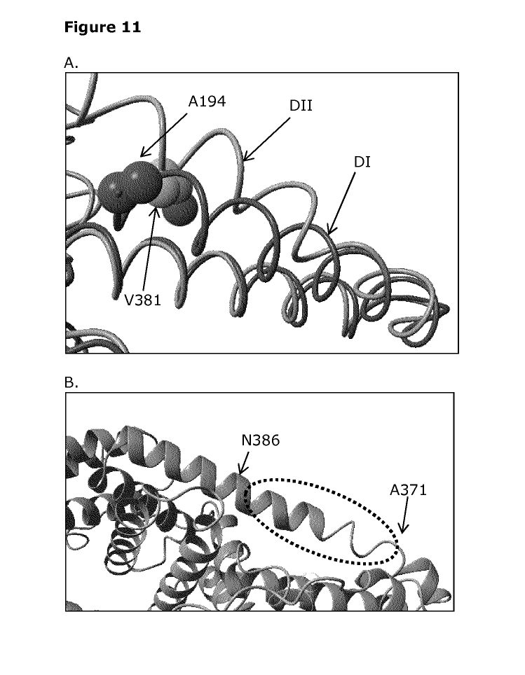

Structural inspection of superimposed Domain I onto Domain II in the context of the full length HSA was used to identify positions which, when subjected to amino acid substitution, insertion or deletion, modulate the binding affinity of albumin to FcRn. For example, such mapping highlighted variation in secondary structural elements at the domain boundaries. These regions are in proximity to Domain III and mutations in these positions in full length HSA may therefore impact shFcRn binding. For example, FIG. 10 shows the variation in secondary structure that would exist in the DI-DIII domain variant compared to the structure of full length HSA. Residues such as L198 and F206 are proximal to Domain III in full length HSA, but there are no positional equivalents in the DI-DIII variant. The absence of such residues may therefore modulate shFcRn binding and therefore alanine variants were explored for positions of this type, e.g. K195A, L198A and F206A (see Example 1, below). In the DI-DIII variant, variation was observed in the disposition of secondary structural elements at the boundary of DI and DIII, at residues 194 and 381 (FIG. 11), which may impact shFcRn binding. Additionally the DII-DIII helix is kinked in this region, resulting from the primary amino acid sequence and this kink is likely altered in the DI-DIII variant. Anchoring the sequence alignment at conserved acidic residues, by manual adjustment of the sequence alignment, permits pairwise comparison to produce variants at positions 377, 378, 379, 380, 381, 382, 383 and 384, such as V381 G (FIG. 8 and FIG. 11).

### Example 2

### Preparation of Variants

### Preparation of Specific HSA Variant Expression Plasmids.

Methods for the expression of HSA variants were performed using several techniques, employing standard molecular biology techniques throughout, such as described in Sambrook, J. and D. W. Russell, 2001 (Molecular Cloning: a laboratory manual, 3^{rd} ed. Cold Spring Harbor Laboratory Press, Cold Spring Harbor, N.Y).

### Method 1

Single amino acid mutations were introduced into the pDB4081 plasmid (encoding WT HSA) using a mutagenic forward primer and non-mutagenic reverse primer (Table 2). pDB4081 was made by the ligation of a synthetic DNA fragment, BsaI/SphI digested, which had been generated by gene assembly (DNA2.0 Inc, USA; SEQ ID 32), containing 3′ region of the PRB1 promoter, modified fusion leader sequence, nucleotide sequence encoding HSA and 5′ region of the modified ADH1 terminator) into HindIII/SphI-digested pDB4005. pDB4005 is described in WO 2011/051489 (incorporated herein by reference). Methylated template DNA was prepared by mixing about 2.5 μg of plasmid DNA with 5 μl 10× buffer (50 mM Tris-HCl mM β-mercaptoethanol, 10 mM EDTA pH 7.5 at 25° C.—New England Biolabs), 1 μl dam methyltransferase (New England Biolabs), 12.5 μl 80 μM s-adenosylmethionine (New England Biolabs) and water to 50 μl final volume and incubating at 37° C. for one hour. Reactions were then purified using a QIAquick PCR purification kit (Qiagen) according to the manufacturer's instructions.

TABLE 2
Oligonucleotides for mutagenic amplification with mutated
codons underlined (R = reverse, F = Forward).

| Oligo | Sequence (5′ to 3′) | SEQ ID NO: |
|---|---|---|
| K195A R | AGCGGAAGAAGCCTTACCTTCGTCTCTCAATTCATC | 33 |
| K195A F | GAAGGTAAGGCTTCTTCCGCTGCTCAAAGATTGAAGTGTGCTTCC | 34 |
| L198A R | TCTTTGCTTAGCGGAAGAAGCCTTACCTTCGTCTCT | 35 |
| L198A F | GCTTCTTCCGCTAAGCAAAGAGCTAAGTGTGCTTCCTTGCAAAAG | 36 |
| F206A R | CTTTTGCAAGGAAGCACACTTCAATCTTTGCTTAGC | 37 |
| F206A F | AAGTGTGCTTCCTTGCAAAAGGCTGGTGAAAGAGCTTTCAAGGCT | 38 |
| D340Y R | TGGGTGTCTTCTAGCGTATTCGTACAAGAACATACC | 39 |
| D340Y F | GAATACGCTAGAAGACACCCATATTACTCCGTTGTCTTGTTGTTG | 40 |
| Y341F R | GTCTGGGTGTCTTCTAGCGTATTCGTACAAGAACAT | 41 |
| Y341F F | TACGCTAGAAGACACCCAGACTTTTCCGTTGTCTTGTTGTTGAGA | 42 |
| S342Y R | GTAGTCTGGGTGTCTTCTAGCGTATTCGTACAAGAA | 43 |
| S342Y F | GCTAGAAGACACCCAGACTACTATGTTGTCTTGTTGTTGAGATTG | 44 |
| V343A R | GGAGTAGTCTGGGTGTCTTCTAGCGTATTCGTACAA | 45 |
| V343A F | AGAAGACACCCAGACTACTCCGCTGTCTTGTTGTTGAGATTGGCT | 46 |
| V344P R | AACGGAGTAGTCTGGGTGTCTTCTAGCGTATTCGTA | 47 |
| V344P F | AGACACCCAGACTACTCCGTTCCATTGTTGTTGAGATTGGCTAAG | 48 |
| L345E R | GACAACGGAGTAGTCTGGGTGTCTTCTAGCGTATTC | 49 |
| L345E F | CACCCAGACTACTCCGTTGTCGAATTGTTGAGATTGGCTAAGACC | 50 |
| R348F R | CAACAACAAGACAACGGAGTAGTCTGGGTGTCTTCT | 51 |
| R348F F | TACTCCGTTGTCTTGTTGTTGTTTTTGGCTAAGACCTACGAAACT | 52 |
| L349F R | TCTCAACAACAAGACAACGGAGTAGTCTGGGTGTCT | 53 |
| L349F F | TCCGTTGTCTTGTTGTTGAGATTTGCTAAGACCTACGAAACTACC | 54 |
| T352R R | CTTAGCCAATCTCAACAACAAGACAACGGAGTAGTC | 55 |
| T352R F | TTGTTGTTGAGATTGGCTAAGAGATACGAAACTACCCTCGAGAAG | 56 |
| F377L R | TTCATCGAAAACCTTAGCGTAACATTCGTGTGGGTC | 57 |
| F377L F | TACGCTAAGGTTTTCGATGAATTGAAGCCATTGGTCGAAGAACCA | 58 |
| K378R R | GAATTCATCGAAAACCTTAGCGTAACATTCGTGTGG | 59 |
| K378R F | GCTAAGGTTTTCGATGAATTCAGACCATTGGTCGAAGAACCACAA | 60 |
| P379D R | CTTGAATTCATCGAAAACCTTAGCGTAACATTCGTG | 61 |
| P379D F | AAGGTTTTCGATGAATTCAAGGATTTGGTCGAAGAACCACAAAAC | 62 |
| L380E R | TGGCTTGAATTCATCGAAAACCTTAGCGTAACATTC | 63 |
| L380E F | GTTTTCGATGAATTCAAGCCAGAAGTCGAAGAACCACAAAACTTG | 64 |
| V381G R | CAATGGCTTGAATTCATCGAAAACCTTAGCGTAACA | 65 |
| V381G F | TTCGATGAATTCAAGCCATTGGGTGAAGAACCACAAAACTTGATC | 66 |
| E382K R | GACCAATGGCTTGAATTCATCGAAAACCTTAGCGTA | 67 |
| E382K F | GATGAATTCAAGCCATTGGTCAAAGAACCACAAAACTTGATCAAG | 68 |
| E383A R | TTCGACCAATGGCTTGAATTCATCGAAAACCTTAGC | 69 |
| E383A F | GAATTCAAGCCATTGGTCGAAGCTCCACAAAACTTGATCAAGCAA | 70 |
| P384S R | TTCTTCGACCAATGGCTTGAATTCATCGAAAACCTT | 71 |
| P384S F | TTCAAGCCATTGGTCGAAGAATCTCAAAACTTGATCAAGCAAAAC | 72 |
| K205A F | TTGAAGTGTGCTTCCTTGCAAGCTTTCGGTGAAAGAGCTTTCAAG | 99 |
| K205A R | TTGCAAGGAAGCACACTTCAATCTTTGCTTAGCGGA | 100 |

The relevant primers were employed in the PCR reaction (described in Tables 3 and 4) using dam-methylated pDB4081 as template and Q5 DNA polymerase (New England Biolabs).

TABLE 3
PCR reaction components

| Template (5 ng/μl) | 1 μl | Forward primer (10 μM) | 2.5 μl |
|---|---|---|---|
| 5x buffer | 10 μl | Reverse primer (10 μM) | 2.5 μl |
| dNTP (2.5 mM) | 1 μl | Q5 polymerase | 0.5 μl |
| Sterile water | 32.5 | | |

TABLE 4
PCR reaction conditions

| Temperature | Cycle Length | Number of cycles |
|---|---|---|
| 98° C. | 2 min | 1 |
| 98° C. | 10 sec | 30 |
| 60° C. | 30 sec | |
| 72° C. | 5 min | |
| 72° C. | 7 min | 1 |

Successful amplification of the plasmid was confirmed by inspection of 5 μl of PCR product on a 1% TBE agarose gel. The remaining PCR product was supplemented with 4 μl buffer 4 (50 mM Potassium acetate, 20 mM Tris-acetate, 10 mM Magnesium acetate, 1 mM DTT, pH 7.9 at 25° C.—New England Biolabs) and 1 μl DpnI enzyme, followed by incubation at 37° C. for one hour. The reactions were then purified using a QIAquick PCR purification kit (Qiagen) according to the manufacturer's instructions. 1 μl of purified plasmid was transformed into E. coli 10-beta cells (New England Biolabs) and plated onto LB plates supplemented with 50 μg/ml ampicillin. Plasmids were isolated using a Qiagen Plasmid Plus Kit (Qiagen—according to manufacturer's instructions) and sequenced to confirm the presence of the desired mutation within the HSA sequence (Table 5).

TABLE 5
Plasmid and amino acid substitutions

| | | SEQ ID |
|---|---|---|
| Variant | Plasmid | NO: |
| K195A | pDB5157 | 73 |
| L198A | pDB5158 | 74 |
| F206A | pDB5159 | 75 |
| D340Y | pDB5160 | 76 |
| Y341F | pDB5161 | 77 |
| S342Y | pDB5162 | 78 |
| V343A | pDB5163 | 79 |
| V344P | pDB5164 | 80 |
| L345E | pDB5165 | 81 |
| R348F | pDB5166 | 82 |
| L349F | pDB5167 | 83 |
| T352R | pDB5168 | 84 |
| F377L | pDB5169 | 85 |
| K378R | pDB5173 | 86 |
| P379D | pDB5229 | 87 |
| L380E | pDB5230 | 88 |
| V381G | pDB5231 | 89 |
| E382K | pDB5232 | 90 |
| E383A | pDB5233 | 91 |
| P384S | pDB5234 | 92 |
| K205A | pDB5156 | 98 |

### Method 2

Combination variants (Table 6) were produced to combine a subset of the mutations described in Table 5 with the HSA K573P mutation (SEQ ID NO: 3). Fragments were removed from plasmids pDB5162, 5165, 5167 and 5231 (Table 5) using the SacII and SalI restriction sites and were purified using a QIAquick Gel Extraction Kit (Qiagen) and ligated into pDB4673 digested with the same enzymes. pDB4673 (HSA K573P) was constructed by insertion of the fragment produced by digestion of pD4283 (described in WO2011/051489, incorporated herein by reference)) with SalI and HinDIII restriction enzymes into similarly digested pDB4081. The ligated plasmids were transformed into E. coli cells, isolated using a Qiagen Plasmid Plus Kit (according to the manufacturer's instructions) and were sequenced to confirm the presence of the desired mutations.

TABLE 6
Plasmid and amino acid substitutions.

| Variant | Plasmid | SEQ ID NO: |
|---|---|---|
| S342Y + K573P | pDB5398 | 93 |
| L345E + K573P | pDB5399 | 94 |
| L349F + K573P | pDB5400 | 95 |
| V381G + K573P | pDB5401 | 96 |

### Production of Expression Plasmid and Yeast Stocks.

Preparation of the expression plasmids and transformation of S. cerevisiae was performed as described in WO2012/150319 (incorporated herein by reference), by either the 24-hour stocking method (pDB5229-5234 and pDB5398-5401) or 48-hour stocking method (pDB5157-5173). The host strain for pDB5229-5234 and pDB5157-5173 was S. cerevisiae DYB7 ura3 (Payne et al. (2008). Applied and Environmental Microbiology Vol 74(24): 7759-7766) with four copies of PDI integrated into the genome. The host strain for pDB5398-5401 was BXP10 Cir⁰. Purification of variants from shake flask was performed as described in WO2012/150319 unless otherwise stated.

### Example 3

### Kinetic Analysis of Binding Affinity of WT HSA and Variants to shFcRn

Kinetic analyses were performed on an Octet Red-96 instrument (ForteBio, Pall Life Sciences) and/or Biacore 3000 instrument (GE Healthcare).

### Kinetic Analyses in Crude Fermentation Supernatants

Kinetic analyses using bio-layer interferometry were performed on an Octet Red-96 instrument. Immobilization of shFcRn was carried out on AR2G biosensors using ForteBio amine coupling chemistry following the instructions from the manufacturer. Immobilized level of shFcRn (either GST-tagged on the C-terminus of the alpha-chain (either obtained from GeneArt, LifeTechnologies or produced in HEK cells according to standard procedures) or his-tagged on the C-terminus of the beta-2-microglobulin (GeneArt, LifeTechnologies)) was at a response level more than 1 nm, and achieved using a FcRn concentration of 2-10 μg/mL in sodium acetate, followed by ethanolamine quenching of the amine coupling reaction. The sensors were either used directly or soaked in sucrose and dried until use.

Kinetic measurements were performed using crude fermentation supernatants from yeast cultures expressing the albumin variants diluted 2-, 4- and 8-fold in fermentation media supplemented with 100 mM sodium acetate and adjusted to pH 5.5. Association (120 s) and dissociation (300 s) were performed at 30° C. and shaking at 1000 rpm. ForteBio software was used for data evaluation and calculation of KD values as well as association and dissociation constants were estimated using the measured HSA concentrations in the supernatants.

### Kinetic Analyses on Purified Variants

With respect to the Biacore 3000 instrument, SPR kinetic analyses were performed as follows: Immobilization was carried out on CM5 chips coupled with shFcRn using GE Healthcare amine coupling chemistry as per manufacturer's instructions. Immobilized levels of shFcRn-HIS (shFcRn with a 6-His tail on the C-terminus of beta-2-microglobulin) were 1200-2000 RU and achieved by injecting 5 μg/mL shFcRn diluted using sodium acetate pH5.0 (G E Healthcare). Chip surface was left to stabilize with a constant flow (5 μL/min) of running buffer—Di-basic/Mono-basic phosphate buffer pH5.5 at 25° C. overnight. After ligand stabilization, the chip surface was conditioned by injecting 6×45 μL Di-basic/Mono-basic phosphate buffer at 30 μL/min followed by HBS_EP (0.01 M HEPES, 0.15 M NaCl, 3 mM EDTA, 0.005% surfactant P20) at pH 7.4 (GE Healthcare)) regeneration steps (12 s) in between each injection. Surfaces were then checked for activity by injecting 6×45 μL positive control at 30 μL/min, followed by 12 s regeneration pulse. Kinetic measurements were performed by injecting dilutions (20 μM-0.0156 μM) of HSA and HSA variants at 30 μL/min over immobilized shFcRn, at 25° C. The reference cell value was then subtracted and Biaevaluation software 4.1 used to obtain kinetic data and confirm KD values.

### Example 4

### Kinetic Analysis of Binding Affinity of WT HSA and Variants to shFcRn

An initial screen of the variants described in Table 7 was carried out using the Octet Red-96 instrument according to the method of Example 3. The data of Table 7 show that alterations at positions 340, 341, 342, 343, 344, 345, 349, 352, 381, 382 and 198, 206, 348, and 383 are potentially important to the interaction between albumin and FcRn and therefore that the presence of an appropriate amino acid at this position would result in an increased binding affinity to FcRn compared to the binding affinity of WT albumin to FcRn.

TABLE 7
Binding affinity of albumin variants to shFcRn

| | SEQ ID | Binding relative |
|---|---|---|
| Variant | NO | to WT HSA |
| K205A | 98 | 0 |
| K195A | 73 | 0 |
| L198A | 74 | − |
| F206A | 75 | − |
| D340Y | 76 | + |
| Y341F | 77 | + |
| S342Y | 78 | + |
| V343A | 79 | + |
| V344P | 80 | + |
| L345E | 81 | + |
| R348F | 82 | − |
| L349F | 83 | + |
| T352R | 84 | + |
| F377L | 85 | 0 |
| K378R | 86 | 0 |
| P379D | 87 | 0 |
| L380E | 88 | 0 |
| V381G | 89 | + |
| E382K | 90 | + |
| E383A | 91 | − |

Binding affinity of variant to shFcRn compared to the binding affinity of WT HSA to FcRn:
+ higher binding affinity,
0 similar binding affinity,
− lower binding affinity

Selected variants showing a higher binding affinity, than HSA, to FcRn were analyzed further using Biacore SPR (Table 8).

TABLE 8
SPR analysis of binding affinity of HSA variants to shFcRn

| | SEQ ID | Ka | Kd | KD | Fold difference |
|---|---|---|---|---|---|
| Variant | NO | 10³/Ms | 10⁻³/s | μM | relative to WT HSA |
| WT* | 2 | 18.0 | 69.8 | 3.9 | — |
| K573P* | 3 | 18.5 | 7.3 | 0.4 | 10 |
| S342Y | 78 | 21.8 | 44.6 | 2.0 | 1.9 |
| | | 20.9 | 45.9 | 2.2 | 1.8 |
| L345E | 81 | 19.6 | 68.5 | 3.5 | 1.1 |
| | | 19.5 | 73.7 | 3.8 | 1.0 |
| L349F | 83 | 28.8 | 26.2 | 0.9 | 4.3 |
| | | 29.5 | 28.0 | 0.9 | 4.3 |
| V381G | 89 | 20.3 | 49.7 | 2.4 | 1.6 |
| | | 26.4 | 48.8 | 1.8 | 2.2 |

*values shown are the mean of duplicates

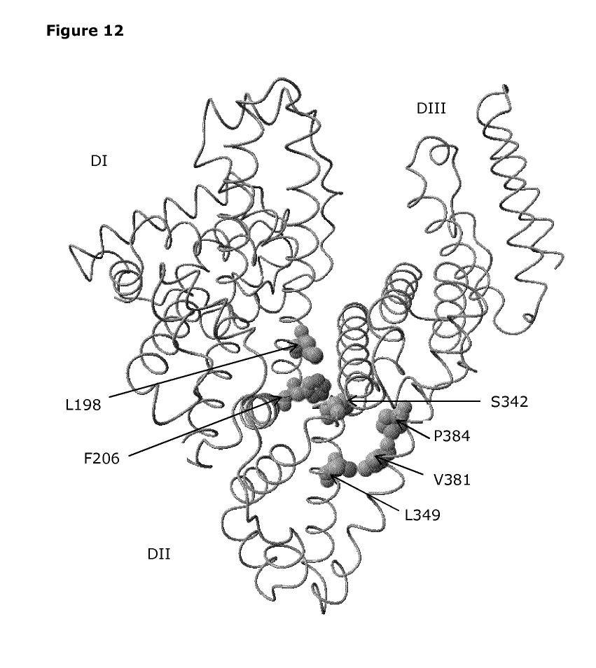

The data of Table 8 show that substitutions at positions 342, 349 and 381 result in improved binding to shFcRn, compared with the binding affinity of WT HSA to shFcRn. Structural inspection of these positions within the molecule shows them to be located within the core of the protein, as opposed to being surface exposed (FIG. 12).

The data of Table 8 and Table 9, in combination with FIG. 12 suggest that an alteration at position P384 will result in a variant having altered binding affinity to FcRn compared to the binding affinity of WT albumin to FcRn.

Albumin variants having the alterations described in Table 8 combined with alteration K573P were constructed and subsequently analyzed by bio-layer interferometry using the Octet Red-96 instrument according to the method of Example 3. The data in Table 9 show that these variants have improved binding to shFcRn compared with the binding affinity of HSA K573P to shFcRn.

TABLE 9
Bio-layer interferometry analysis of
binding affinity of HSA variants to shFcRn

| Variant | SEQ ID NO | Binding relative to HSA-K573P |
|---|---|---|
| S342Y + K573P* | 93 | + |
| L345E + K573P* | 94 | 0 |
| L349F + K573P | 95 | + |
| V381G + K573P* | 96 | + |
| WT* | 2 | − |
| K573P* | 3 | n/a |

*Replicate analyses were carried out. Binding affinity of variant to shFcRn compared to the binding affinity of HSA K573P to FcRn:
+ higher binding affinity,
0 similar binding affinity,
− lower binding affinity,
n/a not applicable.

### Example 5

### Method 3

Combination variants were produced to combine a subset of mutations described in Example 2 (Table 6) with the HSA T83N mutation. Plasmids pDB5398, pDB5400 and pDB5401 were dam-methylated as described in Example 2, Method 1.

These templates and the primers described in Table 10 were employed in PCR reactions, as described in Example 2, Method 1, with the exceptions that 57° C. was substituted for 60° C. as the annealing step, a 5 minute final extension step at 72° C. was used and E. coli DH5alpha cells were used for transformations (New England Biolabs). The resulting plasmids were sequenced to confirm the presence of the desired mutations within the HSA sequence (Table 11).

TABLE 10
Oligonucleotides for mutagenic amplification
with mutated codons underlined

| | | SEQ |
|---|---|---|
| Oligo | Sequence (5′ to 3′) | ID NO: |
| MDH130001 | ACTGTTGCTACCTTGAGAGAAAATTACGGTGAAA | 101 |
| | TGGCTGACTGT | |
| MDH130002 | TTCTCTCAAGGTAGCAACAGTACACAACTTATCA | 102 |
| | CC | |

TABLE 11
Plasmid and amino acid substitutions.

| Variant | Plasmid | SEQ ID NO: |
|---|---|---|
| T83N + S342Y + K573P | pDB5422 | 103 |
| T83N + L349F + K573P | pDB5423 | 104 |
| T83N + V381G + K573P | pDB5424 | 105 |

Preparation of the expression plasmids and transformation of S. cerevisiae (BXP10 Cir⁰) was performed as described In Example 2, Method 2 and the 24-hour stocking method used.

### Method 4

Full permutation libraries were constructed at positions shown to modulate binding of HSA to shFcRn. Therefore, for each of positions 342, 345 and 349, a library of 20 variants was generated. This was done by introducing single amino acid mutations into the pDB5102 plasmid (encoding WT HSA) using a mutagenic forward primer and non-mutagenic reverse primer, for example those shown in Tables 12-14. pDB5102 was made by the ligation of a synthetic DNA fragment, SacII/XhoI digested, which had been generated by gene assembly (GeneArt; SEQ ID 106) into SacII/XhoI-digested pDB4081. The template DNA was dam-methylated as described in Method 1. Mutagenic PCR amplification was performed using the primers described in Tables 12-14 and reactions conditions described in Tables 3 and 4, with the exceptions that 1.25 μl of primers and 35 μl of water were used. Alternative mutations at each position can be produced by alteration of the underlined codon to the appropriate codon for encoding the desired amino acid. Amplification was assessed by agarose gel electrophoresis. 0.5 μl of DpnI (New England Biolabs) was added to each well and the samples were incubated at 37° C. for one hour. 96-well transformation of E. coli cells was performed by the addition of 15 μl DH5a cells and 1 μl of the DpnI-digested PCR reactions to a 96-well plate followed by incubation of ice for 30 minutes. Plates were heat shocked at 42° C. for 40 seconds and placed on ice for 4 minutes. The transformation mix was transferred to a 96-well plate containing 1.25 ml of LB broth supplemented with 50 μg/μl ampicillin, pre-warmed at 37° C. The plate was sealed with gas permeable tape prior to incubation overnight at 37° C., 200 rpm. The plasmid DNA was purified using a Turbo 96 prep kit (Qiagen) according to the manufacturer's instructions. Variants selected for further analysis were sequenced at the mutation site to assess incorporation of the desired mutation. The variant designated HSA23-K was shown to contain lysine at this position, instead of the expected glutamate, presumably due to a polymerase-associated point mutation.

SEQ. ID. NO: 106
CCGCGGAAAACTGTGACAAGTCCTTGCACACCTTGTTCGGTGATAAGTT
GTGTACTGTTGCTACCTTGAGAGAAACCTACGGTGAAATGGCTGACTGT
TGTGCTAAGCAAGAACCAGAAAGAAACGAATGTTTCTTGCAACACAAGG
ACGACAACCCAAACTTGCCAAGATTGGTTAGACCAGAAGTTGACGTCAT
GTGTACTGCTTTCCACGACAACGAAGAAACCTTCTTGAAGAAGTACTTG
TACGAAATTGCTAGAAGACACCCATACTTCTACGCTCCAGAATTGTTGT
TCTTCGCTAAGAGATACAAGGCTGCTTTCACCGAATGTTGTCAAGCTGC
TGATAAGGCTGCTTGTTTGTTGCCAAAGTTGGATGAATTGAGAGACGAA
GGTAAGGCTAGCTCCGCAAAGCAAAGATTGAAGTGTGCTTCCTTGCAAA
AGTTCGGTGAAAGAGCTTTCAAGGCTTGGGCTGTCGCTAGATTGTCTCA
AAGATTCCCAAAGGCTGAATTCGCTGAAGTTTCTAAGTTGGTTACTGAC
TTGACTAAGGTTCACACTGAATGTTGTCACGGTGACTTGTTGGAATGTG
CTGATGACAGAGCTGACTTGGCTAAGTACATCTGTGAAAACCAAGACTC
TATCTCTTCCAAGTTGAAGGAATGTTGTGAAAAGCCATTGTTGGAAAAG
TCTCACTGTATTGCTGAAGTTGAAAACGATGAAATGCCAGCTGACTTGC
CATCTTTGGCTGCTGACTTCGTTGAATCTAAGGACGTTTGTAAGAACTA
CGCTGAAGCTAAGGACGTCTTCTTGGGTATGTTCTTGTACGAATACGCT
AGAAGACACCCAGACTACTCCGTTGTCTTGTTGTTGAGATTGGCTAAGA
CCTACGAAACTACCCTCGAG

TABLE 12
Oligonucleotides for mutagenic amplification of a permutation library at
position 342 with mutated codons underlined
(rev = reverse, all other oligonucleotides are forward orientation)

| Oligonucleotide | Sequence (5′ to 3′) | SEQ ID NO: |
|---|---|---|
| HSAJ-39 | CGCTAGAAGACACCCAGACTACGCTGTTGTCTTGTTGTTGAGATTGGC | 107 |
| HSAJ-40 | CGCTAGAAGACACCCAGACTACTGTGTTGTCTTGTTGTTGAGATTGGC | 108 |
| HSAJ-45 | CGCTAGAAGACACCCAGACTACCACGTTGTCTTGTTGTTGAGATTGGC | 109 |
| HSAJ-46 | CGCTAGAAGACACCCAGACTACATCGTTGTCTTGTTGTTGAGATTGGC | 110 |
| HSAJ-48 | CGCTAGAAGACACCCAGACTACTTGGTTGTCTTGTTGTTGAGATTGGC | 111 |
| HSAJ-50 | CGCTAGAAGACACCCAGACTACAACGTTGTCTTGTTGTTGAGATTGGC | 112 |
| HSAJ-51 | CGCTAGAAGACACCCAGACTACCCAGTTGTCTTGTTGTTGAGATTGGC | 113 |
| HSAJ-52 | CGCTAGAAGACACCCAGACTACCAAGTTGTCTTGTTGTTGAGATTGGC | 114 |
| HSAJ-55 | CGCTAGAAGACACCCAGACTACGTTGTTGTCTTGTTGTTGAGATTGGC | 115 |
| HSAJ-39 rev | GTAGTCTGGGTGTCTTCTAGCGTATTCGTACAAGA | 116 |

*Oligonucleotide “HSAJ-39 rev” was used in combination with each of the forward
oligonucleotides in Table 12.

TABLE 13
Oligonucleotides for mutagenic amplification of a permutation library at
position 345 with mutated codons underlined (rev = reverse)

| Oligonucleotide | Sequence (5′ to 3′) | SEQ ID NO: |
|---|---|---|
| HSAK-07 | CACCCAGACTACTCCGTTGTCCACTTGTTGAGATTGGCTAAGACCTAC | 117 |
| HSAK-08 | CACCCAGACTACTCCGTTGTCATCTTGTTGAGATTGGCTAAGACCTAC | 118 |
| HSAK-13 | CACCCAGACTACTCCGTTGTCCAATTGTTGAGATTGGCTAAGACCTAC | 119 |
| HSAK-01 rev | GACAACGGAGTAGTCTGGGTGTCTTCTAGCGTA | 120 |

*Oligonucleotide “HSAK-01 rev” was used in combination with each of the forward
oligonucleotides in Table 13.

TABLE 14
Oligonucleotides for mutagenic amplification of a permutation
library at position 349 with mutated codons underlined
(rev = reverse, all other oligonucleotides are forward orientation)

| Oligonucleotide | Sequence (5′ to 3′) | SEQ ID NO: |
|---|---|---|
| HSAK-23 | ACTCCGTTGTCTTGTTGTTGAGAGAAGCTAAGACCTACGAAACTACCC | 121 |
| HSAK-32 | ACTCCGTTGTCTTGTTGTTGAGACAAGCTAAGACCTACGAAACTACCC | 122 |
| HSAK-20 rev | TCTCAACAACAAGACAACGGAGTAGTCTGGGTGT | 123 |

*Primer “HSAK-20 rev” was used in combination with each of the forward
primers in Table 14.

Plasmid DNA was prepared for transformation into S. cerevisiae by transfer of 20 μl of plasmid DNA to a 96-well microtiter plate with 30 μl of reaction mix containing 62.5 μl NsiI, 62.5 μl PvuI, 62.5 μl 100×BSA, 625 μl Buffer 3 (100 mM NaCl, 50 mM Tris-HCl, 10 mM MgCl₂, 1 mM DTT pH 7.9 at 25° C.—New England Biolabs) and 2937.5 μl of water. Reactions were incubated at 37° C. for one hour. The host strain was S. cerevisiae DYB7 (Payne et al. (2008) Applied and Environmental Microbiology Vol 74(24):7759-7766). 200 μl of DBY7 cir⁰ competent cells were thawed on ice and mixed with 600 μl sterile 1M sorbitol. 4 μl of NsiI/PvuI-digested plasmid DNA, or similarly digested pDB4081 (WT HSA control), were transferred into 100 ng of Acc65I/BamHI digested pDB3936 DNA (as described in WO 2011/051489, incorporated herein by reference in its entirety) in a 48-well plate. 4 μl of the sorbitol-diluted cells were added directly to each well of the plate, followed by 150 μl sterile-filtered PEG/lithium acetate buffer (80 g PEG 3350 dissolved in water and made up to 200 ml final volume, also including 4 ml 5M lithium acetate). The reaction components were mixed by pipetting and were incubated without shaking at 30° C. for 45 minutes. Plates were then heat shocked at 42° C. for 30 minutes then centrifuged at 3000 rpm for 5 minutes. Supernatants were then removed and 100 μl of sterile 1 M sorbitol added, mixed by pipetting and plates were then centrifuged at 3000 rpm for 5 minutes. The sorbitol was removed and pellets resuspended in 500 μl BMMD+CSM-leu media (described in WO 2012/150319, particularly page 106, incorporated herein by reference in its entirety). Plates were incubated at 30° C. with shaking (200 rpm, 2.5 cm orbit at in a sealed chamber at 100% humidity in an Eppendorf Innova 44 incubated shaker). Transformations for each variant were performed in triplicate, including a WT HSA control (pDB4081). Stocks were produced after four days growth by transfer of 50 μl culture to a fresh microtiter plate containing 50 μl 40% (w/v) trehalose. 50 μl of BMMD+CSM-leu was added prior to a further incubation under the same conditions for 24 hours. Culture supernatants were harvested by centrifugation at 3000 rpm for 5 minutes and 375 μl BMMD+CSM-leu of supernatant transferred to a fresh 48-well microtiter plate. Alternatively, cultures were incubated for 7 days and supernatant harvested as described. Stocks were produced by the addition of 375 μl BMMD+CSM-leu media to the pellet. 50 μl of the resuspended pellet was transferred to a fresh microtiter plate containing 50 μl 40% [w/v] trehalose. Stock plates were stored at −80° C. and supernatant plates at −20° C.

### Example 6

### Further Combination Variants

Variants prepared as described in Example 5, Method 3 and purified according to Example 2, Method 2 were analyzed for binding kinetics. SPR experiments were carried out using a Biacore 3000 instrument (GE Healthcare). Flow cell of CM5 sensor chip was coupled with soluble human FcRn (2000 RU) using amine coupling chemistry as described in the protocol provided by the manufacturer (GE Healthcare). The coupling was performed by injecting 10 μg/ml of the protein in 10 mM sodium acetate pH 4.5 (GE healthcare). Phosphate buffer (67 mM phosphate buffer, 0.15 M NaCl, 0.005% Tween 20) at pH 5.5) was used as running buffer and dilution buffer. Regeneration of the surfaces was done using injections of regeneration buffer i.e. HBS-EP buffer (0.01 M HEPES, 0.15 M NaCl, 3 mM EDTA, 0.005% surfactant P20) at pH 7.4 (GE Healthcare). Post immobilization, the chip was left to stabilize with a constant flow (5 μL/min) of running buffer. Chip surface was conditioned by injecting 6× injections of running buffer followed by 6× injections of regeneration buffer. Surfaces were checked for activity with WT HSA control. For determination of binding kinetics, serial dilutions of albumin variants (10-0.078 μM) were injected over immobilized receptor at a constant flow rate (30 μl/min) at 25° C. In all experiments, data were zero adjusted and the reference cell subtracted. Data evaluations were performed using BIAevaluation 4.1 software (BIAcore AB).

TABLE 15
Kinetic analysis of further combination variants

| | | | | | Fold |
|---|---|---|---|---|---|
| | SEQ | | | | greater |
| | ID | Ka | Kd | KD | than |
| Variant | NO: | (10³/Ms) | (10⁻³/s) | (μM) | WT HSA |
| WT HSA | 2 | 9.2 | 94.7 | 10.0 | — |
| HSA K573P | 3 | 16.0 | 15.6 | 1.0 | 10 |
| HSA T83N + S342Y + K573P | 103 | 18.0 | 9.2 | 0.5 | 20 |
| HSA T83N + L349F + K573P | 104 | 18.8 | 6.5 | 0.3 | 33 |
| HSA T83N + V381G + K573P | 105 | 17.7 | 10.8 | 0.6 | 17 |

### Example 7

### Analysis of Binding Affinities of Variants Produced Via Permutation Libraries at Positions 342, 345, and 349

### Method 5

### Preparation of Variants

Albumin variants were prepared according to Method 4 (above), fermentation supernatants prepared as described in Method 4 and an initial screen for binding affinity to FcRn was carried out using the Method 6 (below). From the initial screen, variants showing greater than two-fold increase in affinity compared to wild type HSA were selected for purification and the binding affinity of these purified variants were analyzed further using Method 7 (below) in order to confirm the greater than two-fold increase in binding affinity to FcRn.

Shake flask culturing of S. cerevisiae and purification was performed as described in WO2012/150319 (incorporated herein by reference) with the following modifications. BMMD media supplemented with 0.69 g/L csm-leu (2×20 mL) was inoculated with S. cerevisiae and grown for 72 h at 30° C. with orbital shaking at 200 rpm. An aliquot of each starter culture (5 mL) was used to inoculate 2×1000 mL BMMD media supplemented with 0.69 g/L csm-leu and grown for 72 h at 30° C. with orbital shaking at 200 rpm. Cells were harvested by filtration through a 0.2 μm vacuum filter membrane (Nalgene Sterile Top Filter) and the supernatant retained for purification.

Albumin variants were purified from culture supernatant using a single chromatographic step using an albumin affinity matrix (AlbuPure™—ProMetric BioSciences, Inc.). Chromatography was performed at a constant linear velocity of 150 cm/h throughout. Culture supernatant (1800 mL) was applied to a 6 cm bed height, 12.5 mL packed bed pre-equilibrated with 50 mM sodium acetate pH5.5. Following load the column was washed with 3 column volume (cv) equilibration buffer then 50 mM ammonium acetate pH8.0 (5 cv). Product was eluted with 50 mM ammonium acetate 10 mM octanoate pH7.0 and the column cleaned with 50 mM ammonium acetate, 30 mM octanoate, 200 mM sodium chloride, pH 7.0 followed by 0.5M NaOH (2cv). Eluate fractions from each albumin variant were concentrated (Vivaspin20 30,000 MWCO PES, Sartorius).

### Method 6

### Kinetic Analyses in Fermentation Supernatants

Kinetic analyses using bio-layer interferometry were performed on an Octet Red-96 instrument (ForteBio, Pall). Immobilization was carried out on Streptavidin sensors using biotinylated human FcRn (biotinylated on the C-terminus of the alpha-chain, purchased from ImmuniTrack Aps) at an FcRn concentration of 5 μg/mL in phosphate buffered saline pH 7.4 supplemented with 0.01% Tween-20. The sensors were pre-blocked with albumin at pH 5.5 and regenerated in phosphate buffered saline pH 7.4 supplemented with 0.01% Tween-20. The streptavidin sensors were either used directly or soaked in sucrose and dried until use.

Kinetic measurements were performed using fermentation supernatants from yeast cultures expressing the albumin variants. The supernatants were 2-fold diluted (preferably 1212 nM, 606 nM and 303 nM) in fermentation media supplemented with 100 mM sodium acetate and adjusted to pH 5.5. Association (120 s) and dissociation (300 s) were performed at 30° C. and shaking at 1000 rpm. Regeneration of the sensors were performed using phosphate buffered saline pH 7.4 supplemented with 0.01% Tween-20. ForteBio software was used for data evaluation and calculation of KD values as well as association and dissociation constants were calculated using the measured HSA concentrations in the supernatants.

### Method 7

### Kinetic Analyses of Purified Variants

Kinetic analyses of purified albumin variants using bio-layer interferometry were performed on an Octet Red-96 instrument. Immobilization was carried out on Streptavidin sensors using biotinylated human FcRn (biotinylated on the C-terminus of the alpha-chain purchased from ImmuniTrack Aps) at an FcRn concentration of 5 μg/mL in phosphate buffered saline pH 7.4 supplemented with 0.01% Tween-20. The sensors were pre-blocked with albumin at pH 5.5 and regenerated in phosphate buffered saline pH 7.4 supplemented with 0.01% Tween-20. The streptavidin sensors were either used directly or soaked in sucrose and dried until use.

Kinetic measurements were performed using the purified albumin variants prepared as 2-fold dilution series from 1515 nM to 24 nM in 25 mM sodium acetate, 25 mM sodium phosphate, 150 mM sodium chloride, 0.01% Tween-20 and adjusted to pH 5.5. Association (120 s) and dissociation (300 s) were performed at 30° C. and shaking at 1000 rpm. Regeneration of the sensors were performed using phosphate buffered saline pH 7.4 supplemented with 0.01% Tween-20. ForteBio software was used for data evaluation and KD's as well as association and dissociation constants were calculated using the measured HSA concentrations.

The variants showing at least two-fold improvement in binding affinity are shown in Table 16.

TABLE 16
Purified HSA variants with greater than 2-fold
improvement in binding over WT HSA.

| Variant | Construct | SEQ. ID NO: |
|---|---|---|
| HSA S342A | HSAJ-39 | 124 |
| HSA S342C | HSAJ-40 | 125 |
| HSA S342H | HSAJ-45 | 126 |
| HSA S342I | HSAJ-46 | 127 |
| HSA S342L | HSAJ-48 | 128 |
| HSA S342N | HSAJ-50 | 129 |
| HSA S342P | HSAJ-51 | 130 |
| HSA S342Q | HSAJ-52 | 131 |
| HSA S342V | HSAJ-55 | 132 |
| HSA L345H | HSAK-07 | 133 |
| HSA L345I | HSAK-08 | 134 |
| HSA L345Q | HSAK-13 | 135 |
| HSA L349K | HSAK-23 | 136 |
| HSA L349Q | HSAK-32 | 137 |

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

What is claimed is:

1. A polypeptide which is a variant of a parent albumin, fragment thereof or fusion polypeptide comprising said variant albumin or a fragment thereof having an altered binding affinity to FcRn compared with the binding affinity of a parent albumin, reference albumin, fragment thereof or fusion polypeptide comprising said parent albumin, reference albumin or fragment or fusion thereof to FcRn, wherein the polypeptide comprises one or more (several) alterations in Domain II of albumin selected from the group consisting of positions corresponding to positions 349, 342, 381, 345, 384, 198, 206, 340, 341, 343, 344, 352, 382, 348, and/or 383 in SEQ ID NO: 2, wherein the polypeptide does not consist of SEQ ID NO: 2 with alteration E382K.

2. The polypeptide according to claim 1 wherein the alteration at the position corresponding to position 349, 342, 381, 345, 384, 198, 206, 340, 341, 343, 344, 352, 382, 348, and/or 383 is a substitution.

3. The polypeptide according to claim 2 wherein the substitution at the position corresponding to position 349 is to F, W Y H P, K or Q.

4. The polypeptide according to claim 2 wherein the substitution at the position corresponding to position 342 is to Y, W, F, H, T, N, Q, A, C, I, L, P, V.

5. The polypeptide according to claim 2 wherein the substitution at the position corresponding to position 381 is to G or A.

6. The polypeptide according to claim 2 wherein the substitution at the position corresponding to position 345 is to E, H, I or Q.

7. The polypeptide according to claim 1 further comprising a substitution at the position corresponding to position 573.

8. The polypeptide according to claim 7 wherein the substitution at the position corresponding to position 573 is to P, Y or W.

9. The polypeptide according to claim 1 further comprising a substitution at the position corresponding to position 83.

10. The polypeptide according to claim 9 wherein the substitution at the position corresponding to position 83 is to N, K or S.

11. The polypeptide according to claim 1 comprising substitutions at positions corresponding to positions 83, 342 and 573 (or equivalent position of other albumins or variants or fragments thereof), preferably T83N, K or S+S342Y, W, F, H, T, N, or Q, A, C, I, L, P, V or Y+K573P, Y or W, more preferably T83N+S342Y+K573P (SEQ ID NO: 103).

12. The polypeptide according to claim 1 comprising substitutions at positions corresponding to positions 83, 349 and 573 of SEQ ID NO: 2 (or equivalent position of other albumins or variants or fragments thereof), preferably T83N, K or S+L349F, W, Y, H, P, K or Q+K573P, Y or W, more preferably T83N+L349F+K573P (SEQ ID NO: 104).

13. The polypeptide according to claim 1 comprising substitutions at positions corresponding to positions 83, 381, and 573 of SEQ ID NO: 2 (or equivalent position of other albumins or variants or fragments thereof), preferably T83N, K or S+V381, G or A+K573P, Y or W, more preferably T83N+V381G+K573P (SEQ ID NO: 105).

14. The polypeptide according to claim 1 wherein the parent albumin or reference albumin is HSA (SEQ ID NO: 2) or a fragment thereof, or a fusion polypeptide comprising HSA or a fragment thereof, most preferably SEQ ID NO: 2.

15. The polypeptide according to claim 1, having a stronger binding affinity to FcRn and/or longer plasma half-life than a parent albumin, reference albumin, fragment thereof or fusion polypeptide comprising said parent albumin, reference albumin or fragment or fusion thereof.

16. The polypeptide according to claim 1, wherein the sequence identity of the polypeptide to SEQ ID NO: 2 is more than 80%, more than 90%, more than 95%, more than 96%, more than 97%, more than 98%, or more than 99%.

17. A fusion polypeptide comprising a polypeptide of claim 1 and a fusion partner polypeptide selected from a therapeutic, prophylactic, diagnostic, imaging or other beneficial polypeptide.

18. A method for preparing a polypeptide which is a variant of albumin, fragment thereof or fusion polypeptide comprising said variant albumin or fragment thereof having a binding affinity to FcRn which is altered compared to the binding affinity of a reference albumin, fragment or fusion thereof to FcRn, comprising:
(a) providing a nucleic acid encoding a parent albumin having at least 80% sequence identity to SEQ ID NO: 2;
(b) modifying the sequence of step (a), to encode a polypeptide which is a variant albumin, fragment thereof or fusion polypeptide comprising said variant albumin or fragment thereof comprising one or more (several) alterations in Domain II of albumin;
(c) introducing the modified sequence of step (b) in a suitable host cell;
(d) growing the cells in a suitable growth medium under condition leading to expression of the polypeptide; and
(e) recovering the polypeptide from the growth medium;
wherein the polypeptide has an altered binding affinity to FcRn and/or an altered plasma half-life compared with the half-life of a parent albumin, reference albumin, fragment thereof or fusion polypeptide comprising said parent albumin, reference albumin or fragment or fusion thereof.

19. The method according to claim 18 wherein the one or more (several) alterations in Domain II of albumin are selected from the positions selected from the group consisting of positions corresponding to 349, 342, 381, 345, 384, 198, 206, 340, 341, 343, 344, 352, 382, 348, and/or 383 in SEQ ID NO: 2, wherein the polypeptide does not consist of SEQ ID NO: 2 with alteration E382K.

20. The method according to claim 19 wherein the alteration at the position corresponding to position 349, 342, 381, 345, 384, 198, 206, 340, 341, 343, 344, 352, 382, 348, and/or 383 is a substitution.

21. The method according to claim 20 wherein the substitution at the position corresponding to position 349 is to F, W Y H P, K or Q.

22. The method according to claim 20, wherein the substitution at the position corresponding to position 342 is to Y, W, F, H, T, N, Q, A, C, I, L, P, V, preferably Y.

23. The method according to claim 20 wherein the substitution at the position corresponding to position 381 is to G or A.

24. The method according to claim 20 wherein the substitution at the position corresponding to position 345 is to E, H, I or Q.

25. The method according to claim 19 further comprising a substitution at the position corresponding to position 573.

26. The method according to claim 25 wherein the substitution at the position corresponding to position 573 is to P, Y or W.

27-50. (canceled)
